(19) United States
(12) Patent Application Publication
(10) Pub. No.: US 2020/0253639 A1
(43) Pub. Date: Aug. 13, 2020

(54) SYSTEMS AND METHODS FOR TISSUE CAPTURE AND REMOVAL

(71) Applicant: Claria Medical, Inc., San Francisco, CA (US)

(72) Inventors: Steven W. KIM, Los Altos, CA (US); Joseph N. JONES, Boise, ID (US); Alexey SALAMINI, San Francisco, CA (US); Scott ANDERSON, Sunnyvale, CA (US); Ronald G. French, San Jose, CA (US)

(21) Appl. No.: 16/758,358

(22) PCT Filed: Oct. 22, 2018
(86) PCT No.: PCT/US2018/056915

§ 371 Date: Apr. 22, 2020

Related U.S. Application Data
(60) Provisional application No. 62/576,019, filed on Oct. 23, 2017.

(51) Int. Cl. A61B 17/42; A61B 17/34

(52) U.S. Cl. CPC A61B 17/4241; A61B 2017/320775; A61B 17/3423

(57) ABSTRACT

Components, systems and kits for capturing and removing tissue from mammalian bodies include a tissue container that may be introduced into a body cavity and within which a tissue specimen may be placed, cut and removed from the body cavity. Methods of using these components, systems and kits are also described.

The present application is a national stage application under 35 U.S.C. section 371, which claims the benefit of priority to PCT Application No. PCT/US2018/056915, having a filing date of Oct. 22, 2018, titled “SYSTEMS AND METHODS FOR TISSUE CAPTURE AND REMOVAL”, which claims priority from U.S. Provisional Application No. 62/576,019, filed Oct. 23, 2017, naming Steven W. Kim et al. as inventors, titled “SYSTEMS AND METHODS FOR TISSUE CAPTURE AND REMOVAL,” each of which is hereby incorporated in its entirety by reference.

### BACKGROUND

In the field of health care in human and veterinary medicine, it is often desirable or even necessary to remove tissue from a patient's body. Such tissue, typically in the form of mass, tumor, or organ, some of which may be cancerous, pre-cancerous, or be suspected of being cancerous or pre-cancerous, may be removed via traditional surgical techniques, including open surgery and minimally invasive approaches.

Among minimally invasive approaches, laparoscopic procedures in which a tissue specimen is removed via a small incision using specialized tools are well known. Minimally invasive procedures such as laparoscopy and mini-laparotomy may also employ the use of tools operated robotically. Among procedures performed via minimally invasive techniques include those performed in the abdominal, pelvic and thoracic cavities. Cholecystectomies, nephrectomies, colectomies, hysterectomies, and other procedures in gastrointestinal, gynecological and urological categories are common as are minimally invasive arthroscopy, cystoscopy, and thoracoscopy procedures. Among the various advantages cited with minimally invasive procedures include reduced pain, lower risk of infection, shorter recovery times, and lower cost, among others.

Often, the tissue specimen to be removed via minimally invasive procedures is larger than the incisions used. As such, techniques have been developed to safely remove such specimens while maintaining the advantages of a minimally invasive approach. One such technique is morcellation, in which the tissue specimen is cut or processed into pieces while still inside the patient so that they may be more readily removed. Morcellation historically has been accomplished manually via traditional surgical approaches (i.e. not via minimally invasive approaches), with the physician or other user operating morcellators by squeezing a handle or the like; even direct cutting of the tissue specimen via a scalpel or other instrument through the surgically-created tissue orifice, such as a surgical incision, vaginal cuff, etc. is performed. Power morcellation, in which a morcellation device operated by electricity or other means, is another commonly employed technique.

In the field of gynecology, the hysterectomy is a common procedure that is performed in approximately 500,000 women per year in the United States alone. It involves removing a woman's uterus for a variety of reasons, most commonly because of the presence of uterine fibroids. Such hysterectomies may be performed via traditional open surgical techniques or minimally invasive techniques, such as laparoscopy with the use of morcellation. Hysterectomies may be partial, involving removal of, e.g., only the uterus, or total, in which the uterus and uterine cervix are both removed. In either case, the ovaries and/or the fallopian tubes may or may not simultaneously be removed.

For years, power morcellation has been used in gynecologic surgery to remove large uteri from patients via small holes, as is necessary in minimally invasive surgery. The most common application of power morcellation in gynecologic surgery involved morcellating a large, fibroid uterus to remove it from a patient's body during robot-assisted total laparoscopic hysterectomy, although there are a number of other applications as well.

Since hysterectomy involving an enlarged uterus is very common, and since minimally invasive surgery offers many benefits to the patient, surgeon, hospital, and payer, the use of power morcellation had become commonplace. However, the potential for occult cancers hidden within the uterus that cannot be detected preoperatively and that could potentially be spread around the patient's body with grave consequences during morcellation has been a source of concern. As such, even though most hysterectomies are associated with uteri that do not involve any actual or suspected cancer, traditional open surgery, with its added risk, complication rates, longer hospitalizations, more difficult recoveries, etc., is prevalent.

Therefore, techniques and systems are desirable that afford safe removal and processing of tissue specimens, even in the possible presence of an occult malignancy.

In approaching this problem, systems and methods of the present disclosure improve the safety, speed, ease of use, and efficiency of the tissue removal process via minimally invasive approaches, both in gynecological and non-gynecological applications.

### SUMMARY

The present disclosure embodies various methods, component, systems and kits for capturing and removing tissue from mammalian bodies.

In one embodiment, a method of the present disclosure includes introducing at least a portion of a tissue container into a patient's pelvic cavity through the patient's vagina, placing a tissue specimen into an interior of the tissue container, removing at least a portion of the tissue container from the pelvic cavity through the vagina such that an edge defining an opening in the tissue container is outside the vagina, introducing a cutter into the container interior through the vagina, cutting at least a portion of the tissue specimen with the cutter; and removing the tissue specimen from the container interior and out of vagina through the cutter. A cannula may be introduced at least partially into the container interior through a central lumen of the cannula. In addition, a tissue grasper may be released at least partially into the container interior through either or both the cannula central lumen or a central lumen of the cutter. The tissue grasper may be used to grasp at least a portion of the tissue specimen prior to or during the step of cutting at least a portion of the tissue specimen with the cutter. The tissue grasper may be introduced at least partially into the container interior through the vagina. The step of grasping at least a portion of the tissue specimen may include drawing the tissue specimen into contact with a blade of the cutter prior to or during the cutting step. A guard may be deployed within the container interior, prior to or concurrently with the step of introducing the cutter, to protect the tissue container from damage. The guard may be expandable from a collapsed configuration such that when the guard is deployed within the container interior it expands into a cone shape. The cutter may comprise a guard for protecting the tissue container from damage prior to or during the cutting step and may also comprise a protector portion having at least one protector element. The cannula may comprise a protector portion having an asymmetric extension, or the cannula may include a protector portion comprises an enclosing element at least partially covering the protector element and/or the asymmetric extension. In this method, at least a portion of the tissue container may be removed from the pelvic cavity through the vagina such that tissue specimen is thereby moved in apposition to or near the cutter. Tension may be applied to at least a portion of the tissue container prior to or concurrently with cutting at the portion of the tissue. This tension can be applied by an operator physically applying tension on the container by hand, by pulling on one or more tethers attached to the container, by a twisting motion that shortens an axial length of the container, and/or by an automated system. The tissue specimen can include at least one of a uterus, ovary, and fallopian tube. In addition, the method can employ at least one laparoscopic instrument that is introduced through one or more ports and into the pelvic cavity to prepare and/or visualize the tissue specimen prior to the step of placing the tissue specimen container interior. The laparoscopic instrument may also be used to place or assist placing the tissue specimen into the tissue container.

One embodiment includes a tissue containment and removal system having an expandable tissue container with an interior, a tissue cutter having a distal end that is at least partially disposable within the container interior and a guard that is deployable within the container interior and over the cutter distal end such that the guard is between the container interior and the cutter. The system can also include a cannula at least partially disposable within the container interior. The cannula can have a central lumen through which the cutter may be disposed. The guard may be partially collapsible and expandable into a cone shape upon deployment within the container interior. The system can also include a tissue grasper that is at least partially disposable within the container interior and/or at least partially disposable within and axially movable through the cannula lumen. The tissue grasper can be at least partially disposable within and axially movable through a central lumen of the tissue cutter.

One embodiment includes a tissue containment and removal system having an expandable tissue container with an interior, a tissue cutter having a distal end that is at least partially disposable within the container interior and a cannula at least partially disposable within the container interior. The cannula can have a main portion, a protector portion and a central lumen through which the cutter may be disposed. The cannula protector portion can comprise at least one protector element and/or at least one asymmetric extension. The cannula protector portion can further include an enclosing element at least partially covering the protector element and/or the asymmetric extension. The system can also include a tissue grasper that is at least partially disposable within the container interior and/or at least partially disposable within and axially movable through the cannula lumen. The tissue grasper can be at least partially disposable within and axially movable through a central lumen of the tissue cutter.

One embodiment includes a tissue containment and removal system having an expandable tissue container with an interior, a tissue cutter having a distal end that is at least partially disposable within the container interior and a cannula at least partially disposable within the container interior. The cannula can have a central lumen through which the cutter may be disposed. The system can also include a guard that is deployable within the container interior and over the cutter distal end such that the guard, when deployed, is disposed between the container interior and the cutter. The guard may comprise at least one protector element and/or an asymmetric extension and/or an enclosing element at least partially covering the at least one protector element and/or the asymmetric extension. The system may also include a tissue grasper at least partially disposable within and axially moveable through the container interior and/or at least partially disposable within and axially movable through the cannula lumen. The tissue grasper may also be at least partially disposable within and axially movable through a central lumen of the tissue cutter.

One embodiment includes a tissue containment and removal system having an expandable tissue container with an interior, and a tissue cutter having a distal end that is at least partially disposable within the container interior, the cutter comprising a main portion and a protector portion. The cutter protector portion can include at least one protector element and/or an asymmetric extension and/or an enclosing element at least partially covering the at least one protector element. The system may also include a tissue grasper at least partially disposable within the container interior and which may be at least partially disposable within and axially movable through the cannula central lumen. The system may also include a cannula at least partially disposable within the container interior and having a central lumen through which the tissue cutter may be disposed. The tissue grasper may also be at least partially disposable within and axially movable through the cannula central lumen and/or through a central lumen of the tissue cutter.

One embodiment includes a tissue containment system having a collapsible tissue container with at least one opening, a closure mechanism, and at least one reinforcing member selected from the group consisting of a reinforcing member having a curved cross-sectional profile, a reinforcing member that extends radially outward relative to a central longitudinal axis of the container, and a reinforcing member that is a helically-shaped expansion spring. The container can be impermeable to the transmission or leakage of biological cells, and can be a composite structure. The container can also be a bi-layer structure. If the container is a composite or a bi-layer structure, a first inner layer may be present that is resistant to cutting and puncturing, such as, e.g., poly-paraphenylene terepthalamide. One or more tethers may also be part of the container and may be affixed to the container. The closure mechanism may be selected from the group comprising a zipper, a tongue and groove closure, a clasp, a string tie, a hook and loop fastener, a clasp, a drawstring, and a drawstring with a reinforcing member. A closure member can be included which is operable to move a zipper mechanism to close the at least one opening. The closure mechanism may be operated from a location outside the body of a patient when the container is disposed at least partially there within. The container opening may generally be circular, and an edge of the container near the opening can have at least one stiffening member. The tissue container opening can also generally be triangular and an edge of the container near the opening can have two stiffening members. The system can also include a container tensioning mechanism. The tensioning mechanism can be operable by a hand crank or by an automated system comprising a motor and a programmable control module. The container may be deployed in a radial fashion by the manipulation of one or more wires to create a container interior into which a tissue specimen may be placed, and motion of the one or more wires around an approximate 360 degree path allows an edge of container to mate with itself to close the container. The system may also include a handle disposed near the container opening. The handle may be integrally formed with the container or it may be configured to be attached to the container by a user.

In one embodiment, a method of tissue removal includes the steps of introducing at least a portion of a tissue container into a body cavity through a body port, placing a tissue specimen into an interior of the tissue container, removing at least a portion of the tissue container from the body cavity such that an edge defining an opening in the tissue container is outside the port, introducing a cutter into the container interior through the port, cutting at least a portion of the tissue specimen with the cutter, and removing the tissue specimen from the body cavity through the cutter. The body cavity may be a pelvic cavity and the tissue specimen is one or more tissue specimens selected from the group consisting of a uterus, a fallopian tube, and an ovary. The body port may be selected from the group consisting of a surgical incision, a trocar, and a vagina. The body cavity can also be an abdominal cavity and the tissue specimen can be selected from the group consisting of solid and hollow viscera found within the abdominal cavity, including without limitation small intestines, large intestines, colon, rectum, liver, bladder, omentum, abdominopelvic sidewalls, and any other abdominal organ or any solid or cystic tumor or lesion associated with any of the foregoing. The body cavity can also be a thoracic cavity and the tissue specimen can be selected from the group consisting of solid and hollow viscera found within the thoracic cavity, including without limitation cardiac tissue, lungs, bronchi, other pulmonary tissue, esophageal tissue, vessels, lymph-associated tissue, and any other thoracic organ or any solid or cystic tumor or lesion associated with any of the foregoing. The body cavity can also be a retroperitoneal space and the tissue specimen may be selected from the group consisting of solid and hollow viscera found within the retroperitoneal space, including without limitation kidneys, adrenal glands, spleen, ureters, muscles, vessels, lymph associated tissue, and any other retroperitoneal organ or any solid or cystic tumor or lesion associated with any of the foregoing.

In one embodiment, a method for isolating and removing tissue from a mammalian body includes the steps of inserting a cannula through a at least partially through a tissue port to an ostium of a tissue cavity, deploying a specimen bag through the cannula into the tissue cavity, placing a tissue specimen into an interior of the bag with a tissue grasper, the grasper having been deployed at least partially into the tissue cavity through the cannula or through a second tissue port, deploying a guard through the cannula into the bag interior, inserting a cutter through the cannula into an interior space of the guard proximal to a distal end of the guard, moving the tissue specimen against a blade of the cutter with the tissue manipulator, actuating the cutter while applying tension on the bag against the tissue cavity surface such that the tissue specimen is at least partially dissected, distally retracting the cutter and tissue manipulator from the bag, closing the bag, removing the closed bag containing the at least partially dissected tissue specimen by distally retracting the bag through the cannula, and removing the cannula from the tissue port.

One embodiment includes a tissue containment and removal kit having an expandable tissue container with an interior, a tissue cutter comprising a distal end that is at least partially disposable within the container interior, a guard that is deployable within the container interior and over the cutter distal end such that the guard is between the container interior and the cutter, and instructions for use.

One embodiment includes a tissue containment and removal kit having an expandable tissue container with an interior, a tissue cutter comprising a distal end that is at least partially disposable within the container interior, a guard that is deployable within the container interior and over the cutter distal end such that the guard is between the container interior and the cutter, and instructions for use.

One embodiment includes a tissue containment and removal kit having an expandable tissue container with an interior, a tissue cutter comprising a distal end that is at least partially disposable within the container interior, a cannula disposable within the container interior, the cannula having a central lumen through which the cutter may be disposed, a guard that is deployable within the container interior and over the cutter distal end such that the guard is between the container interior and the cutter, and instructions for use.

One embodiment includes a tissue containment and removal kit having an expandable tissue container having an interior, a tissue cutter comprising a distal end that is at least partially disposable within the container interior, the cutter comprising a main portion and a protector portion, and instructions for use.

In one embodiment, a method of capturing and removing tissue includes introducing at least a portion of a tissue container into a patient's pelvic cavity through a laparoscopic port, placing a tissue specimen into an interior of the tissue container, removing at least a portion of the tissue container from the pelvic cavity through the laparoscopic port such that an edge defining an opening in the tissue container is outside the laparoscopic port, introducing a cutter into the container interior through the laparoscopic port, cutting at least a portion of the tissue specimen with the cutter, and removing the tissue specimen from the container interior and out of the laparoscopic port through the cutter. The method may also include the step of introducing a cannula at least partially into the container interior, wherein the cutter is introduced into the container interior through a central lumen of the cannula. The method may also include the steps of introducing a tissue grasper at least partially into the container interior through either or both the cannula central lumen or a central lumen of the cutter and grasping at least a portion of the tissue specimen with the tissue grasper prior to or during the step of cutting at least a portion of the tissue specimen with the cutter. The method may also include the steps of introducing a tissue grasper at least partially into the container interior through the laparoscopic port and grasping at least a portion of the tissue specimen with the tissue grasper prior to or during the step of cutting at least a portion of the tissue specimen with the cutter. When grasping at least a portion of the tissue specimen, the method can mean this to include drawing the tissue specimen into contact with a blade of the cutter prior to or during the cutting step. The method may further include the step of deploying a guard within the container interior, prior to or concurrently with the step of introducing the cutter, to protect the tissue container from damage. The cutter may comprise a guard for protecting the tissue container from damage prior to or during the cutting step. The cannula may include a protector portion having at least one protector element and/or an asymmetric extension. The cannula protector portion may also include an enclosing element at least partially covering the at least one protector element and/or the asymmetric extension. The guard may be expandable from a collapsed configuration such that when the guard is deployed within the container interior it expands into a cone shape. The method may also include the feature that wherein when at least a portion of the tissue container is removed from the pelvic cavity through the laparoscopic port, the tissue specimen is thereby moved in apposition to or near the cutter. Further, the method may include the step of applying tension to at least a portion of the tissue container prior to or concurrently with cutting at the at least one portion of the tissue. This tension may be applied by an operator physically applying tension on the container by hand, by pulling on one or more tethers attached to the container, by a twisting motion wherein the twisting motion shortens an axial length of the container. This tension may also by a semi-automated or an automated system. The tissue specimen can include at least one of a uterus, ovary, and fallopian tube. At least one laparoscopic instrument can be introduced through one or more additional laparoscopic ports and into the pelvic cavity; such instrument may be used in the method to prepare and/or visualize the tissue specimen prior to the step of placing the tissue specimen container interior. This laparoscopic instrument can also be used to place or assist placing the tissue specimen into the tissue container.

In one embodiment, a method of tissue removal includes the steps of introducing at least a portion of at least one tissue container into a body cavity through a body port, placing a tissue specimen into an interior of the at least one tissue container, removing at least a portion of the at least one tissue container from the body cavity such that an edge defining an opening in the at least one tissue container is outside the port, introducing a cutter into the at least one container interior through the port, cutting at least a portion of the tissue specimen with the cutter, and removing the tissue specimen from the body cavity through the cutter. The method may further include the step of applying tension to at least a portion of the at least one tissue container prior to or concurrently with cutting at the at least one portion of the tissue. Such tension may impart a force on the tissue specimen to bring the specimen in apposition with the cutter. One embodiment includes a tissue containment and removal system that is capable of performing any of the steps of this method.

In one embodiment, a method of tissue removal includes the steps of introducing at least a portion of a tissue container into a body cavity through a body port, placing a tissue specimen into an interior of the tissue container, removing at least a portion of the tissue container from the body cavity such that an edge defining an opening in the tissue container is outside the port, introducing a cutter and a locking member into the container interior through the port, cutting at least a portion of the tissue specimen with the cutter, and removing the tissue specimen from the body cavity through the cutter. The method may include embodiments in which the locking member is an inflatable balloon and further includes the step of inflating the balloon prior to the cutting step. The method may also include the step of applying tension to at least a portion of the tissue container prior to or concurrently with cutting at the at least one portion of the tissue, and this tension may impart a force on the tissue specimen to bring the specimen in apposition with the cutter. The method may also include the step of introducing a cannula into the container interior through the port, and such cannula may be introduced prior to or simultaneously with the introduction of the cutter. The cutter may be introduced through a central lumen of the cannula. The locking member can be disposed on the cannula, and the locking member may be an inflatable balloon. The locking member may be configured to prevent contact between the container and the cutter. The method may further include the step of inflating the balloon prior to the cutting step. The balloon may anchor the cannula to a portion of the body cavity prior to or concurrent with the step of removing the tissue specimen. One embodiment includes a tissue containment and removal system that is capable of performing any of the steps of this method.

Some embodiments of a tissue containment and removal system may include a container configured to contain and isolate a tissue specimen. Such a container may include an interior volume, an opening having an edge, a distal portion disposed opposite the opening, a wall portion including a layer of thin, flexible, fluid tight material, an insufflation manifold extending along the wall portion from the edge into the interior volume towards the distal portion and including an insufflation manifold lumen disposed within the insufflation manifold and at least one manifold port in fluid communication between the insufflation manifold lumen and the interior volume of the container, and a tab secured to and extending radially outward from an outer surface of the container. The system may also have a morcellator including a distal end that is at least partially disposable within the interior volume of the container and a morcellator cannula having a central lumen extending an axial length of the morcellator cannula.

Some embodiments of a tissue containment and removal system may include a container configured to contain and isolate a tissue specimen. Such a container may include an interior volume, an opening having an edge, a distal portion disposed opposite the opening, and a wall portion including a layer of thin, flexible, fluid tight material. The system may also have an access sheath that includes a sheath cannula having a proximal end and a distal end, a central lumen extending axially through a length of the sheath cannula, and a locking member disposed on a distal end of the sheath cannula. The system may further include a morcellator having a morcellator cannula which is slidably disposable within the central lumen of the sheath cannula, a central lumen extending an axial length of the morcellator cannula, and a distal end that is at least partially disposable within the opening and interior volume of the container.

Some embodiments of a tissue containment and removal system may include a container configured to contain and isolate a tissue specimen. The container may include an interior volume, an opening having an edge, a distal portion disposed opposite the opening, and a wall portion including a layer of thin, flexible, fluid tight material. The system may further include a source of pressurized gas having an outlet port and a first insufflation tube having an inner lumen in fluid communication between the outlet port and an interior volume of a patient's body cavity. The system may also have a second insufflation tube having an inner lumen in fluid communication between the outlet port and the interior volume of the container. A pressure regulator valve may be disposed in the second insufflation tube between the interior volume of the container and the outlet port, the pressure regulator valve being configured to restrict the flow of pressurized gas therefrom until a predetermined threshold pressure has been reached in the interior volume of the container.

Some embodiments of a method of capturing and removing tissue transvaginally may include introducing at least a portion of a container into a patient's abdominal cavity through the patient's vagina and placing a tissue specimen into an interior volume of the container. An edge of the container may be withdrawn from the abdominal cavity through the vagina such that the edge defining an opening in the container is disposed outside the vagina. A distal end of a morcellator may be introduced into the interior volume of the container through the vagina and the interior volume of the container insufflated by supplying pressurized gas into an insufflation manifold lumen of an insufflation manifold which extends along a wall portion from the edge into the interior volume towards a distal portion of the container and emitting pressurized gas from at least one manifold port of the insufflation manifold which is in fluid communication between the insufflation manifold lumen and the interior volume of the container. Thereafter, at least a portion of the tissue specimen may be morcellated with the morcellator and the tissue specimen removed from the interior volume of the container and out of vagina through a central lumen of a cannula of the morcellator.

Some embodiments of a method of capturing and removing tissue transvaginally may include introducing at least a portion of a container into a patient's abdominal cavity through the patient's vagina and placing a tissue specimen into an interior volume of the container. An edge of the container may be withdrawn from the abdominal cavity through the vagina such that the edge defining an opening in the container is disposed outside the vagina. A distal end of a morcellator may be introduced into the interior volume of the container through the vagina and the interior volume of the container insufflated by supplying pressurized gas into an insufflation lumen of a cannula of the morcellator which extends from a proximal end of the cannula to a distal end of the cannula and emitting the pressurized gas from an insufflation outlet port which is in fluid communication between the insufflation lumen and the interior volume of the container. Thereafter at least a portion of the tissue specimen may be morcellated with the morcellator and the tissue specimen removed from the interior volume of the container and out of vagina through a central lumen of the cannula of the morcellator.

Some embodiments of a method of capturing and removing tissue transvaginally may include introducing at least a portion of a container into a patient's abdominal cavity through the patient's vagina and placing a tissue specimen into an interior volume of the container. An edge of the container may be withdrawn from the abdominal cavity through the vagina such that the edge defining an opening in the container is disposed outside the vagina. A distal end of an access sheath may be introduced into the interior volume of the container through the vagina and the interior volume of the container insufflated by supplying pressurized gas into an insufflation lumen of a cannula of the access sheath which extends from a proximal end of the cannula to a distal end of the cannula and emitting the pressurized gas from an insufflation outlet port which is in fluid communication between the insufflation lumen and the interior volume of the container. A distal end of a morcellator may be introduced through a central lumen of the cannula of the access sheath and into the interior volume of the container and at least a portion of the tissue specimen morcellated with the morcellator. The tissue specimen may then be removed from the interior volume of the container and out of vagina through a central lumen of a cannula of the morcellator.

Some embodiments of a tissue containment and removal system may include a container configured to contain and isolate a tissue specimen. The container may include an interior volume configured to contain a tissue specimen, an opening having an edge, a distal portion disposed opposite the opening, a wall portion including a layer of thin, flexible, fluid tight material. The system may further include a pressure regulator valve which is disposed on the wall portion of the container, which is disposed in fluid communication between the interior volume of the container and a position exterior to the container and which is configured to restrict a flow of pressurized gas out of the interior volume of the container until a predetermined threshold pressure has been reached in the interior volume of the container.

Some embodiments of a tissue containment and removal system 1010 may include a tissue container having an interior volume, and a wall portion having a composite multiple layer structure that comprises a first layer including a polymer and a second layer disposed inside of the first layer which includes a high strength cut and puncture resistant material. The system may also include a morcellator having a housing with a rigid configuration, a tissue cutter that has a tubular configuration, a distal end, a central lumen, a cutter blade disposed on the distal end, and an outer dimension that is at least partially disposable within the interior volume of the tissue container. The morcellator may also include a drive which is disposed within the housing and which is configured to rotate the tissue cutter relative to the housing when operated. The system may further include a cannula which has a tubular configuration, which is at least partially disposable within the interior volume of the tissue container and which includes a central lumen that is configured for axial passage and rotation of the tissue cutter. A releasable mount may also be optionally included with the system which is configured to releasably secure a proximal end of the cannula to the housing in both a protected position with the distal end of the cannula extending distally over the cutter blade with the cutter blade being covered by the distal end of the cannula and a cutting position with the distal end of the cannula disposed in a position which is disposed axially proximal of the cutter blade with the cutter blade exposed for tissue cutting and morcellation.

Some embodiments of a method of capturing and removing tissue transvaginally may include introducing at least a portion of a container into a patient's pelvic cavity through a vagina of a patient. Such a container may include an interior volume, a wall portion having a composite multiple layer structure that includes a first layer including a polymer and a second layer disposed inside of the first layer which includes a high strength cut and puncture resistant material. A tissue specimen from within the patient's pelvic cavity may be placed into the interior volume of the container and an edge of an opening of the container removed from the pelvic cavity through the vagina such that the edge surrounding the opening in the container is disposed outside the vagina. A distal end of a cannula which has a tubular configuration and a central lumen that extends an axial length thereof may be inserted into the interior volume of the container so as to provide a conduit into the interior volume of the container from outside the vagina. A tissue cutter of a morcellator may be inserted through the central lumen of the cannula and into the interior volume of the container. A proximal end of the cannula may be releasably secured to a housing of the morcellator such that the axial position of the tissue cutter is substantially fixed with respect to an axial position of the cannula. Such releasable securing of the proximal end of the cannula to the housing may take place either before or after insertion of the cannula into the interior volume of the container. Thereafter, at least a portion of the tissue specimen may be cut with a cutter blade of the tissue cutter and the tissue specimen removed from the interior volume of the container and out of vagina through a central lumen of the cutter.

### BRIEF DESCRIPTION OF THE DRAWINGS

FIG. 1 is a perspective view of an embodiment of a system for tissue capture and removal, not including a tissue container for clarity.

FIG. 2 is a perspective view of the components of the FIG. 1 system embodiment in exploded form.

FIG. 3A is a partial perspective section view of an embodiment of a tissue container.

FIG. 3B is a partial perspective section view of an embodiment of a tissue container including stiffening or reinforcing members.

FIG. 4 is a cross-sectional schematic of a composite tissue container incorporating a bi-layer construction.

FIGS. 5A-C are various views of an embodiment of a tissue container including bi-stable spring stiffening or reinforcing members.

FIGS. 6A-C are partial perspective views of two embodiments of a tissue container closure mechanism.

FIGS. 7A-B are various views of embodiments of a tissue container incorporating expandable spring mechanisms or features.

FIGS. 8A-C depict the operation of a two-part tissue container.

FIGS. 9A-D depict the operation of a tissue container configured to encircle a tissue specimen.

FIGS. 10A-C depict the operation of a tissue container that in use may initially be collapsed, presented through a cannula or port, used to capture a specimen via a circular opening and then closed automatically through the same cannula or port.

FIG. 11 depicts the capture mode operation of a tissue container similar to that of FIGS. 10A-C utilizing a triangular opening to capture a specimen instead of a circular opening.

FIGS. 12A-C depict the operation of a tissue container utilizing a twist-to-tension feature.

FIGS. 13A-C depict the operation of a tissue container utilizing a telescoping-to-tension feature.

FIGS. 14A-C depict the operation of a tissue container utilizing a camera-like twist-to-tension feature.

FIGS. 15A-D depict the operation of a tissue grasper having tines.

FIGS. 16A-B depict a cannula integrated with a guard or protector portion having flexible/expandable protector elements.

FIGS. 17A-C are various views of a cannula integrated with an asymmetric guard or protector portion and flexible/expandable protector elements encased in a protective material.

FIGS. 18A-D are various views of an end of the cannula of FIGS. 17A-CF.

FIG. 19 is a side view of an embodiment of a system for tissue capture and removal featuring a hand-operated cutting mechanism.

FIG. 20 depict a method of use of an embodiment of a system for tissue capture and removal including a pullback tensioning feature.

FIG. 21 depicts various views of cutter embodiments according to the present disclosure.

FIGS. 22A-B illustrate in schematic form various dimensional relationships among tissue components of a system of tissue capture and removal.

FIGS. 23A-F illustrate a method of use of a system of tissue capture and removal.

FIGS. 24A-F illustrate another method of use of another system of tissue capture and removal.

FIGS. 25A-C illustrate yet another method of use of a further system of tissue capture and removal.

FIGS. 26A-D schematically depict another embodiment of the present disclosure and method of use.

FIG. 27 illustrates various locking member embodiments that may be used with embodiments of the present disclosure.

FIGS. 28A-C depict embodiments employing one or more balloons and two separate containers.

FIG. 29 is a perspective view with hidden lines shown of a container embodiment.

FIG. 30 is a transverse cross section view of the container embodiment of FIG. 29.

FIG. 30A is an enlarged view in section of encircled portion 30A shown in FIG. 30.

FIG. 30B is a top view of an embodiment of the container of FIG. 29 in a flattened state.

FIG. 30C is a perspective view of a container embodiment.

FIG. 30D is a transverse cross section view of the container of FIG. 30B.

FIG. 31 is a perspective view of a container embodiment that includes a plurality of stiffeners that are circular in shape and have flexible resilient properties.

FIG. 32 is an elevation view in longitudinal section of the container embodiment of FIG. 31.

FIG. 33 is a perspective view of a container embodiment that includes a stiffener having a spiral configuration with flexible resilient properties.

FIG. 34 is an elevation view in longitudinal section of the container embodiment of FIG. 33.

FIG. 35 is a perspective view of the stiffener of the container embodiment of FIG. 33.

FIG. 36 is a perspective view in partial section with hidden lines shown of a container embodiment that includes a stiffener having a lattice configuration and resilient flexible properties.

FIG. 37 is an elevation view in longitudinal section of the container embodiment of FIG. 36.

FIG. 38 is an elevation view of the stiffener embodiment of the container of FIG. 36.

FIGS. 39-41 illustrate an embodiment of a double wall container that may be used for isolating and removing tissue from a patient's body.

FIG. 42 is a schematic illustration of an insufflation system embodiment.

FIG. 43 is a perspective view of a patient being prepared for a minimally invasive tissue removal procedure prior to being reclined into a position such as a trendelenberg position.

FIG. 44 is a top view of an abdominal portion of the patient of FIG. 43 illustrating an example of port positions through a wall of the patient's abdomen.

FIGS. 45-47 are views in longitudinal section of the abdomen portion of the patient shown in FIG. 44 during a tissue removal procedure.

FIG. 48 is an enlarged view in longitudinal section of a distal section of a laparoscope embodiment shown in FIG. 47.

FIG. 49 is an enlarged view in longitudinal section of the distal section of the laparoscope embodiment of FIG. 47 being docked into a laparoscopic dock embodiment of a container embodiment.

FIG. 50 shows a distal end of the laparoscope embodiment docked within a laparoscope channel of the laparoscopic dock embodiment of FIG. 49.

FIG. 51 is an elevation view in longitudinal section of the abdominal portion of the patient with an embodiment of a container disposed about a uterine tissue specimen and with atraumatic graspers coupled to tabs of the container.

FIG. 52 is a perspective view of a tenaculum embodiment disposed within a central lumen of a morcellator embodiment.

FIG. 53 is an elevation view of the morcellator and tenaculum embodiments of FIG. 52.

FIG. 54 is a transverse cross section view of the morcellator and tenaculum embodiments of FIG. 53.

FIG. 55 is an elevation view in partial section of the morcellator and tenaculum of FIG. 54.

FIG. 56 is an enlarged view of distal sections of the morcellator and tenaculum of FIG. 55.

FIG. 57 is a view in longitudinal section of the patient's abdomen portion with a container embodiment disposed about a uterine tissue specimen and with an interior volume of the container in an insufflated state and with a plurality of gripper devices applying outward tension on respective tabs of the container.

FIG. 58 shows the system of FIG. 57 with a portion of the tissue specimen being proximally drawn into a central lumen of a cannula of the morcellator with the tenaculum.

FIG. 59 shows a transverse cross section view of the system for containment and retrieval of tissue and the patient's abdominal portion of FIG. 58.

FIG. 60 is a perspective view of an access sheath embodiment.

FIG. 61 is an elevation view of the access sheath embodiment of FIG. 60 with a locking member disposed on a distal portion thereof shown in a deflated state.

FIG. 62 is a section view of an abdominal portion of the patient with the access sheath of FIG. 61 being deployed into an interior volume of a container embodiment with an atraumatic tipped obturator disposed within a central lumen of the access sheath and with the locking member in a deflated state.

FIG. 63 is an elevation view in longitudinal section of the access sheath of FIG. 61 showing a first seal and second seal disposed within a proximal section of a central lumen of a cannula of the access sheath.

FIG. 64 shows a morcellator embodiment being introduced into the central lumen of the access sheath with a seal being formed between an outside surface of a cannula of the morcellator and the first seal and second seal of the access sheath.

FIG. 65 is an enlarged view of a proximal portion of the access sheath as shown in FIG. 64.

FIG. 66 is a section view of an abdominal portion of the patient with the access sheath deployed in interior volume of container with the locking member in an inflated and expanded state.

FIG. 67 is a perspective view of a morcellator embodiment, cannula embodiment and tenaculum embodiment.

FIG. 68 is an exploded view of the morcellator embodiment of FIG. 67.

FIG. 69 is a perspective view of an embodiment of a tab receiver of a releasable mount embodiment.

FIG. 70 is a perspective view of the cannula embodiment of FIG. 67.

FIG. 71 is a perspective view of an embodiment of an obturator.

FIG. 72 is a perspective view of the morcellator embodiment of FIG. 67 with the obturator of FIG. 71 disposed therein.

FIG. 73 is a perspective view of the morcellator embodiment of FIG. 67 with the cannula disposed in a protected position.

FIG. 74 is a perspective view of the morcellator embodiment and tenaculum of FIG. 67 with the cannula disposed in a cutting position.

FIG. 75 is an elevation view in section of the morcellator and cannula embodiment of FIG. 67 disposed within an interior volume of the container embodiment of FIG. 29 within a patient's body cavity and with the obturator of FIG. 71 disposed within the morcellator.

FIG. 76 is an elevation view in section of the patient's body cavity of FIG. 75 with the tenaculum of FIG. 67 disposed within the morcellator.

### DETAILED DESCRIPTION

The following description should be read with reference to drawings in which similar elements in different drawings are numbered the same. The drawings, which are not necessarily to scale, depict illustrative embodiments and are not intended to limit the scope of the present disclosure.

Embodiments of the present disclosure are fundamentally different than any previous iteration of tissue access and removal involving morcellation, particularly power morcellation: in the context of a hysterectomy, for example, embodiments disclosed herein are the first that may be deployed into the pelvic or pelvic cavity through the vagina, once the uterus and cervix have been dissected off the top of the vagina. In contrast, previous power morcellators have only been used through an abdominal laparoscopic port 862, which renders them not only cumbersome, difficult, and awkward to operate, but is limited to removing tissue pieces whose maximum size is that of the port, typically on the order of about 12.0 mm in diameter. Embodiments of the present disclosure have the advantage that they can be deployed, in the gynecologic context, trans-vaginally, thus affording a physician or other user the ability to remove tissue pieces as large as the surgical opening in the vagina itself, typically on the order of about 30.0 to about 60.0 mm in diameter. The number of “passes”, or cycles of tissue cutting or morcellation needed to remove a specimen, can therefore decrease from dozens to a handful, with concomitant savings in total operative time & patient anesthesia exposure (and corresponding costs).

As such, embodiments of the present disclosure allow for ready tissue specimen capture within an enclosure such as a container or bag, relatively simple and safe tissue cutting/processing/morcellation within the bag, and a design that protects the container from being breached by the tissue cutter/morcellator or other instrument. Indeed, outside the transvaginal context, smaller versions of systems described herein can be deployed via a pelvic, abdominal or other laparoscopic port for use in applications where no vaginal access is possible.

In general, system embodiments of the present disclosure can could consist of one component, two distinct components, three distinct components or more, or a combination of 2 or 3 or more distinct components. A particular function may be, in some embodiments, performed by different components or multiple components operating together, depending on the system configuration and the particular application for which that configuration is designed.

A two-part system could consist of a specimen container and a tissue cutting device, for example. The container can generally be leak-proof and impermeable to cells, liquids, gases, etc., and can function to prevent the spread of cancerous or otherwise dangerous biological materials into the patient's body cavity during the act of specimen removal. The container can include features that protect the surrounding healthy tissue from being damaged by accidental contact with the cutter or other instrument and that enable swift and efficient specimen containment. The tissue cutter or morcellator can safely interface with the container for the purpose of removing the specimen from the patient's body. Using one configuration of such a two-part system, a physician or other operator can deploy a container into the patient's body cavity, capture and place the tissue specimen therein, and then mate the morcellator/cutter to the container for specimen cutting and removal. In another embodiment, the container and cutter are a single unit.

In another example, a third part consisting of a tissue grasper or tenaculum is built into a system that includes the cutter, which will be mated to the container or bag. Thus the cutter and the manipulator can be one assembly and the container can be a separate mating component. Alternatively, a third part consisting of a tissue grasper can be built into a system that includes both the cutter and the bag, and all three components exist as a single unit.

A four part system may consist of a tissue container, a tissue cutter, a tissue grasper and a tissue manipulator. In gynecology applications, a tissue manipulator typically is termed a uterine manipulator (such as the VCARE DX uterine manipulator sold by ConMed Corporation of Utica, N.Y.) and is often used to detach the uterus or specimen from the body. In gynecological applications of the present disclosure, a physician or other user employs a combination system such as a four part system by seating itself inside the vagina. First, a cuff of the uterine manipulator is seated around the patient's cervix and a manipulator arm is extended into the interior of the uterus prior to uterine detachment. Once the uterus is detached using means knows to those of skill in the art, the uterine manipulator is extracted and the cutter, tissue grasper and tissue container are introduced. The uterine manipulator and the tissue cutter can both share the same port on the device. The tissue specimen is then captured in the container, reduced in size through cutting, and removed from the body along with the tissue cutter, tissue grasper, and tissue container.

In general, the tissue container, tissue cutter, tissue grasper and tissue manipulator, as well as other components of systems described below, in their various configurations, may be made available in a variety of sizes so as to accommodate differences in patient size and anatomy.

A feature of embodiments disclosed herein include mechanisms and techniques by which the container can be maintained under tension or traction during use, in some cases constant tension, thus minimizing its size within the patient's body cavity. As will be described herein, this may be achieved by, e.g., simple pulling on the container by a physician or other operator during use, by way of a self-tensioning mechanism, or by incorporation of a powered or non-powered crank, ratchet, rolling or other means.

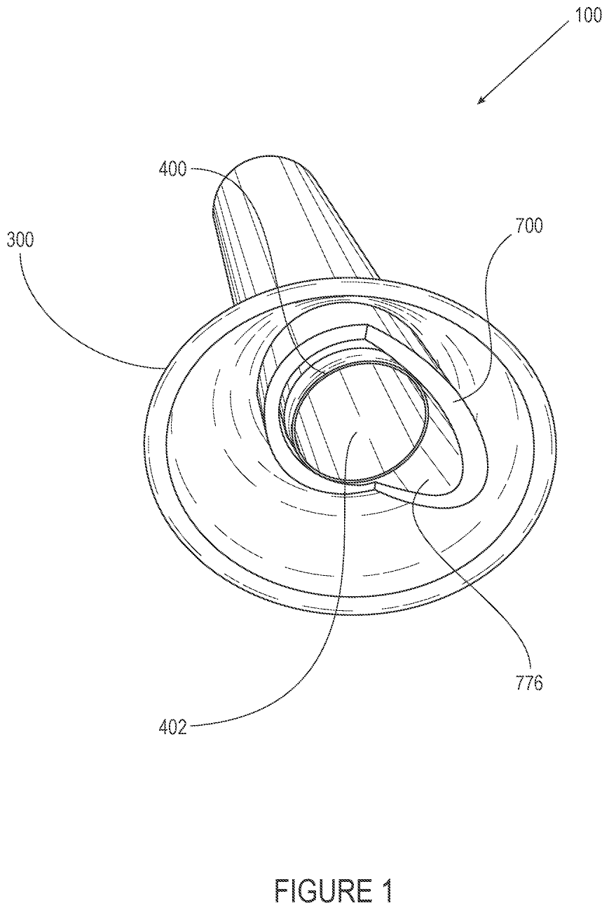

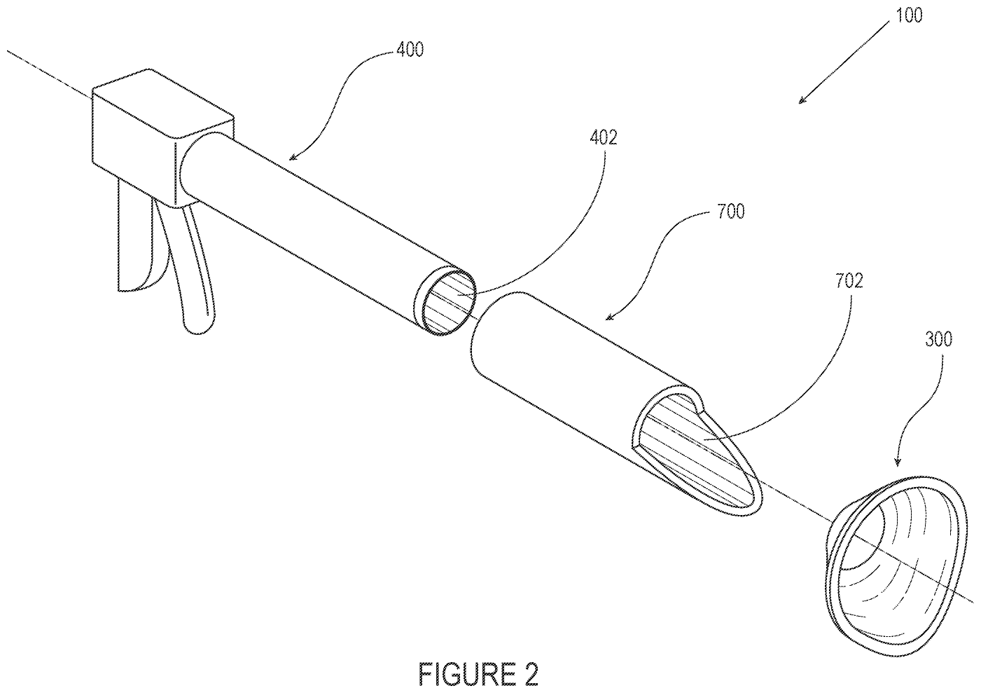

FIGS. 1 and 2 depict some components of a system 100 of tissue capture and removal according to an embodiment of the present disclosure. In the assembled FIG. 1 perspective view, guard 300, cutter or morcellation device 400 and cannula 700 are shown in working relationship to one another, while the exploded perspective view of those components in FIG. 2 affords a more detailed examination of each. For clarity, neither FIG. 1 nor FIG. 2 includes a tissue container 200 or a tissue grasper 500. Tissue grasper 500 may be deployed through a central lumen 402 of cutter 400. Tissue grasper 500 and/or cannula 700 may be omitted from systems of the present disclosure, such that in some embodiments system 100 consists of a cutter 400, a guard 300 (and/or protective feature that may be integrated with one or more components described herein), and a container or bag 200. In other embodiments, system 100 may include a cutter or morcellator 400 and a container 200. The example system 100 of FIGS. 1 and 2 includes both a guard 300 and a protective feature in the form of extension 776 on cannula; one or both of these features may be included in various embodiments of systems of the present disclosure.

As will be more fully described below, system 100 may be used for the safe and efficient access to, capture, and removal of tissue from a human or other mammalian body. Embodiments of system 100 and other instruments, such as standard laparoscopic and robotic instruments, gas injectors for insufflation, and visualization tools such as cameras, etc., as described herein may be used in particular in connection with minimally invasive procedures, such as those undertaken laparoscopically, where the tissue specimen of interest to be removed is relatively large compared to the size of the port. The port, sometimes referred to herein as a “body port” or “opening” may be a surgically created incision, including without limitation various pelvic or abdominal incisions (such as umbilical, periumbilical, left and/or right lower quadrant, left upper quadrant, etc.), appliances or devices that may be installed in a body, such as subcutaneously, including as dermal ports, venous ports, arterial ports and the like trocars, and incisions from prior surgeries or procedures. The port or body port may also be a natural body opening (e.g., vagina, rectum, esophagus, nostrils/nasal canal, bronchial tubes, auditory canal, etc.) through which the specimen 20 is to be removed. Embodiments of the systems 100 of the present disclosure and various components discussed herein can be used in connection with any of these surgically-created or natural ports or via any combination of two or more of such ports.

The components discussed herein, including those of system 100 as shown in FIGS. 1 and 2, may be sized and constructed of materials appropriate to the location of the tissue specimen 20, the indication, the particular port or opening through which the specimen is to be removed, patient size, etc.

System 100 may include a cannula 700 having a central lumen 702702 through which may be disposed a tissue grasper or forceps, (e.g., a tenaculum) or similar instrument 500 for the manipulation of tissue; particularly tissue specimen 20, to be removed. Typically, but not always, cannula 700 if used is deployed through a tissue enclosure/container or bag 200 that has previously been deployed through a body lumen or port as described in detail below. A cutter or morcellation device 400 may be disposed through cannula central lumen 702 for the processing of tissue specimen 20 as shown in the system 100 embodiment of FIGS. 1 and 2. An optional spacer (not shown) may be disposed in or be an integral part of cutter central lumen 402 aids in keeping or serves to keep grasper 500 centered within lumen 402. A guard 300 may be employed to protect container 200 from damage as the tissue specimen 200 is processed by cutter 400 and protect tissue not intended for removal (e.g., bowels, bladder or other tissues depending on the location of treatment). Guard 300 may be a cone-shaped component as shown in FIGS. 1-2 or may take on another shape as will be described below. Guard 300 and/or its function may be attached to or even integrated with other components of systems described herein, including, e.g., cutter 400, grasper 500, cannula 700, or combinations thereof.

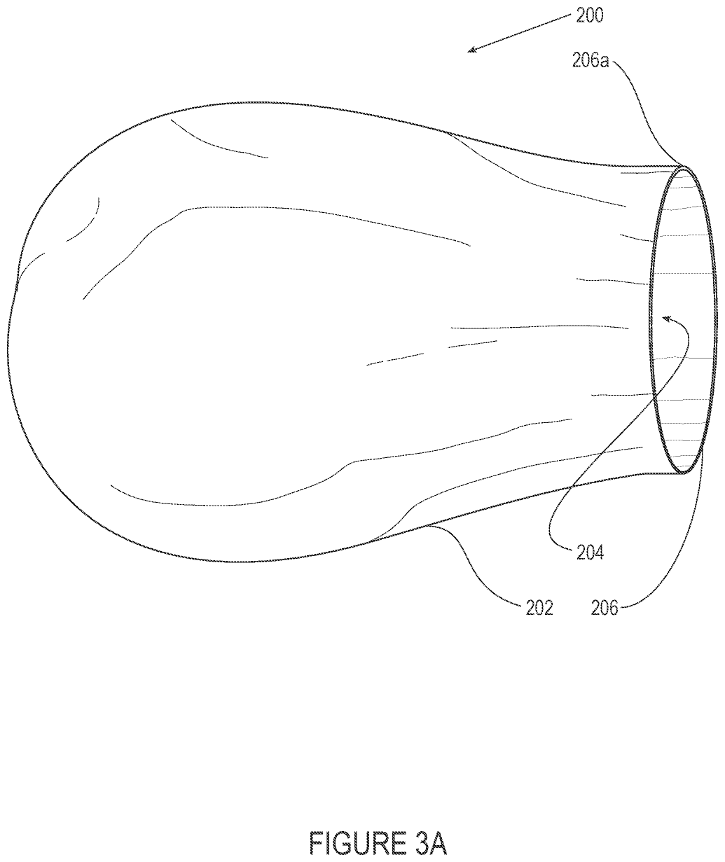

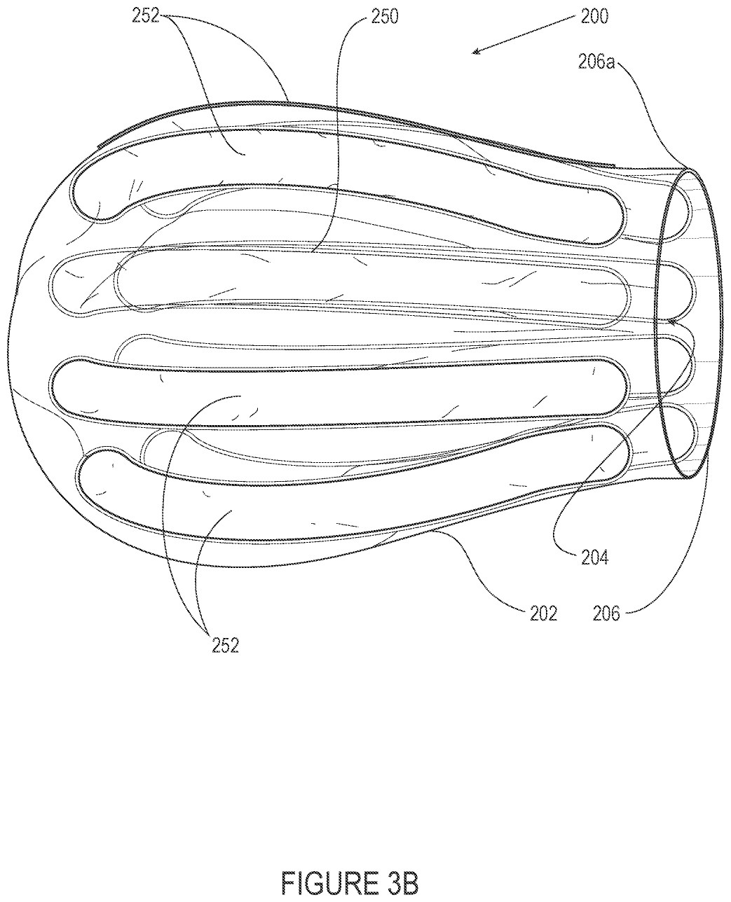

FIGS. 3A-B illustrate two embodiments of an enclosure apparatus, or tissue container 200, according to the present disclosure. In general, the primary function of tissue container or bag 200 is safely to contain one or more tissue specimens or samples 20 during the procedure or method of use for the systems described herein. Container 200 may be more rigid or stiff, or less rigid or stiff (akin to that of a bag); thus, the terms “container”, “enclosure” and “bag” are used interchangeably herein to encompass all embodiments useful to achieve the purposes of this disclosure. Such terms therefore encompass flexible or deformable bags, semi-rigid bags or containers, rigid or non-deformable containers, containers having both relatively rigid and relatively flexible components or aspects, and the like. Bag 200 may take on any shape and size suitable for the indication for which it is designed. For instance, enclosure 200 may take on a generally cylindrical, spherical, spheroidal (e.g., prolate spheroid), prismatic, pyramidal, cuboid, cubical, conical, irregular (e.g., pear, squash, etc.) or otherwise asymmetrical shape or a hybrid of two or more of these fundamental shapes. If used in a gynecological procedure where the uterus and/or other organs are to be placed therein, tissue container 200 may be in a generally spherical shape and have diameters ranging from between about 50.0 mm or less by about 400 mm or greater. Container 200 may also be in a generally spherocylindrical shape (i.e., pill capsule) or a semi- or hemi-spherocylindrical shape with an opening diameter of between about 50.0 mm or less to about 400 mm or greater, such that a tissue specimen (e.g., uterus) having its greatest dimension on the order of generally about 20.0 cm to about 30.0 cm or larger may be placed and stored therein. A hemi-spherocylindrical shape may be useful in systems where the bag opening 206 is large. If the container 200 is measured in terms of three dimensions, container 200 may take on sizes ranging from about 300 mm by 300 mm by 400 mm. For other indications, such as the capture and retrieval of a stomach mass via the esophagus, bag 200 may take on any of the aforementioned shapes and have a size ranging from those useful in removing a uterus or smaller.

Bag 200 includes an outer surface 202, an interior volume 204, and at least one opening or aperture 206 defined by one or more edges such as edge 206a shown in FIGS. 3A-B. In one embodiment, bag 200 is impermeable to cancer cells, yet is thin and flexible enough so that it may be rolled, folded or otherwise compacted so that it may be transported through a small port, hole, lumen or other aperture, typically on the order of about 5.0 mm or smaller to about 25.0 mm or greater in diameter. Bag 200 can also be made to withstand tears, punctures, impacts and generally undesirable interactions with surgical instruments, tools (including, e.g., robotic and/or laparoscopic tools) and other components of system 100 (including, e.g., cutter 400 and its blade 408), etc. In this way, container 200 can operate to maintain its structural and functional integrity to safely keep one or more tissue samples 20 placed within it properly isolated from the environment outside the bag, such as may exist in an abdominal or pelvic cavity before container 200 containing specimen is removed from the patient's body 31. This is particularly useful in designs where the tissue specimen 20 placed in the container 200 contains cancerous or pre-cancerous cells or is suspected of containing cancerous or pre-cancerous cells.

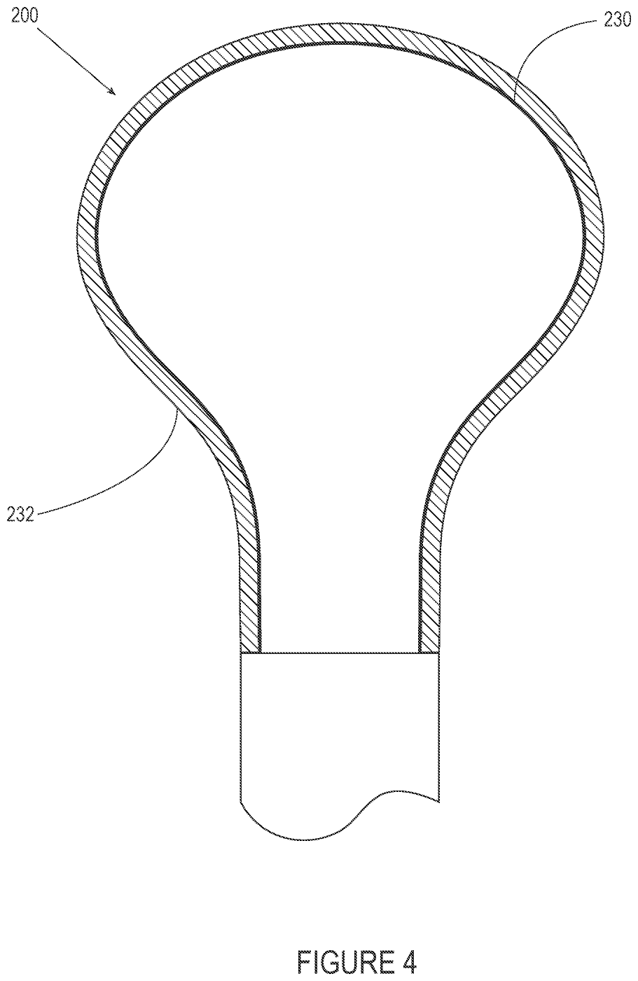

Tissue container 200 may be made of any suitable biocompatible material, including plastics such as polyethylene, polyurethane, polypropylene, PET, PETG, aramid and para-aramids, including, e.g., poly-paraphenylene terepthalamide (KEVLAR), aliphatic or semi-aromatic polyamides (NYLON), rubber, thermoplastics and others. It may be of a composite construction, including a bi-layer construction as shown in the example embodiment of FIG. 4. Such composite embodiments may be made from, e.g., two sheets of material that are folded flat, as multiple sheets and formed into a three-dimensional bag shape, or can, e.g., be manufactured in a three-dimensional fashion by use of a molding or special tool and/or by way of blow molding, compression molding, or three-dimensional printing techniques. Constructing bag 200 from different materials may pose advantages from durability, toughness, usability, cost, manufacturing, marketing or other perspectives. For instance, bag 200 may be made of a visually transparent or opaque plastic layer or layers so to allow a physician or surgeon to visualize tissue specimens as they are placed and/or after they have been placed into bag 200 and to allow visualization through the container to see tissue on the other side. This is particularly useful when using fiber optic or other camera or video equipment during a procedure. One embodiment of a composite container 200 can be created through the construction of multiple layers of plastic and mesh. In the bilayer container embodiment depicted in FIG. 4, each layer can serve different or overlapping purposes: one layer, such as an outer layer 232, can create a watertight seal for the contents of the bag while another layer, such as an inner layer 230, can protect the container from the morcellator blade. Both layers together, for example, combine to provide the desired toughness, puncture- and tear resistance, etc. properties as discussed herein. Inner layer 230 can be made out of a durable plastic such as those in the aramid and para-aramid classes, including, e.g., poly-paraphenylene terepthalamide, or can be made of or incorporate a metal mesh to protect the container from the blade.

Bag 200 may be doped by known techniques to render it, e.g., radiopaque for optimal utility in certain applications, it may contain wires, filaments, or other materials to cause the bag to change shape, radiate electromagnetic signals, thermally activate, or chemically transform as desired. It may also come pre-treated with one or more agents to affect the tissue specimen if desired, such as a preservative agent, contrast agent, etc., and/or may be coated with one or more layers of hydrophilic or hydrophobic materials and/or other lubricating materials or otherwise treated to provide a low-friction environment for the bag interior 204 with which tissue specimen 20 will be in contact. Such coatings or layers may be discrete and applied during manufacturing in sequential fashion (e.g., three-dimensional printing, other known deposition techniques) or may be in a composite or alloy-like form during manufacturing and/or as-fabricated. Having a low-friction and/or lubricious surface, particularly in bag interior 204 can facilitate methods of tissue cutting and removal according to embodiments described herein, as tensioning of container 200 tends to bring tissue specimen 20 within close proximity of or in direct contact with container interior 204 and the cutting process may benefit as the specimen 20 can spin or otherwise move relatively easily against the interior surface of bag 200. A “peeling” process in particular as a way of cutting tissue 200 may benefit from such a container configuration under the methods described herein. Container 200 may in some embodiments contain markings such as gradations or a grid pattern (such as employed on the PNEUMOLINER containment device sold by Olympus America, Inc. of Southborough, Mass.) to aid the physician in locating and assessing the size of tissue samples placed therein, ascertaining whether the container 200 is folded or crimped in some way, how much of the container is left inside the body as it is being removed by, e.g., rolling edge 206a when applying tension on container, etc. Such markings may be present using Cartesian coordinates, radial coordinates, or spherical coordinates depending on the shape, configuration and contemplated use or uses for container 200.

As will be described below in detail with respect to several embodiments of the present disclosure, container 200 can have a tether, drawstring, or other component affixed thereto or integrated therewith such that a physician or other user may manipulate the bag during use, facilitating its placement, opening, closing, and removal from the body. In one embodiment, one or more tethers extend(s) from the container 200 in the vicinity of opening 206, and attached, integrated or otherwise affixed on or near container edge 206a. Such tethers may be stiff, particularly with respect to their column stiffness, or they may be more flexible. The use of a tether or similar component is useful in procedures where the enclosure 200 has been deployed into the body cavity 30 of interest for placement of a tissue specimen 20 into the bag's interior 204, and the tether or tethers extend(s) out of the body cavity 30 through the access port 22 or natural opening (e.g., vagina, esophagus, etc.) and held, affixed or tied to a separate instrument and/or simply monitored so that at the appropriate time during the procedure the physician or other user may pull on the tether or tethers to safely and effectively remove the bag from the patient's body 31 through opening 22. Tethers may also be utilized to aid a physician, either manually or via the use of automated equipment, in applying and/or maintaining tension on enclosure 200 during the tissue capture and removal process.

One embodiment of container 200 includes one or more stiffeners or reinforcement members 252, each of which can be initially separate from container 200, as shown in FIG. 3B. Features such as stiffeners 252 allow container 200 to assume and maintain a desired shape or volume at the appropriate time to aid in the tissue capture and removal process and can also help to prevent the cutter 400, including blade 408, from damaging container 200. Any number of stiffeners of identical or varying dimensions, shapes and materials may be used. For instance, between 1 and 4, between 2 and 8, between 4 and 16 or more stiffeners may be used. Any medical grade material having the appropriate mechanical properties may be used for stiffeners 252, such as spring steel, certain plastics, nickel titanium alloys, etc. Each stiffener could be a composite material; for instance, a bilayer construction that imparts preferential stiffness under bending forces for one direction compared to another may be useful. Certain embodiments of stiffener 252 may be shape-set using techniques known to those of skill in the art to undergo strain- or temperature-induced transformations during manufacturing, packaging and/or use to optimize performance. A given stiffener may have a thickness ranging from about 0.01 mm to about 4.0 or more mm, and may have a longitudinal dimension (when shaped as shown in FIG. 3B) of between about 1.0 cm and about 20 cm or longer, and widths ranging from about 1.0 mm to about 30 mm or more. Stiffeners 252 can be arranged symmetrically to facilitate the desired performance of the enclosure 200 or, in certain situations, may be configured to be arranged with an asymmetric distribution or pattern to coax container 200 to take on a particular shape when open and/or to force or at least facilitate certain sequential motions of the container 200 during use, including opening the container, closing the container, fastening the container, tensioning the container and removing the container from the patient's body 31.

One or more pockets 250 on or in container 200 may be present to guide the insertion of and house the stiffeners 252, either partially or completely, therein. Stiffeners 252 may be inserted during the tissue capture and removal procedure by the physician or other user (e.g., after container has been deployed into the pelvic cavity 30) or they may be manufactured as an integral part of or attached to tissue container 200. Stiffeners 252 may be sized and have the flexibility to allow the bag to be rolled into a small dimension for insertion through a surgical port or natural body opening 22. In other embodiments, stiffeners 252 could be directed through one or more loops or other fastening mechanisms that keep the stiffeners 252 between a morcellation blade 408 and container 200.

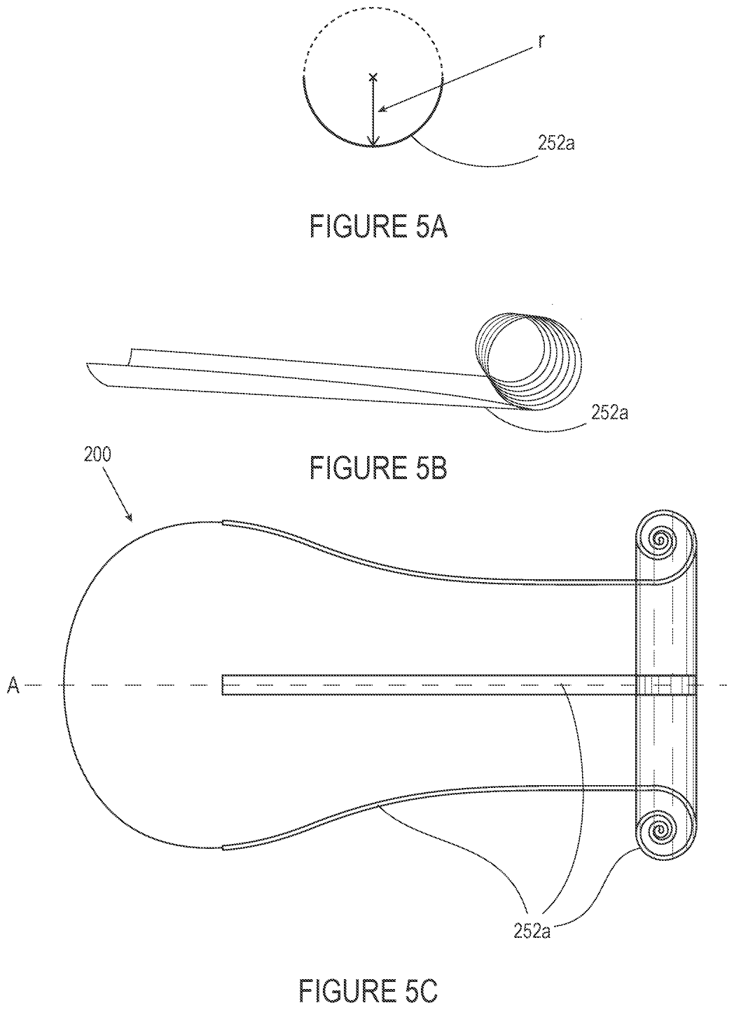

FIGS. 5A-C show another embodiment in which the stiffener 252a can take on a curved cross-sectional profile such as a ‘U’ shape (FIG. 5A), T shape, “V” shape, etc. to provide stiffness or resistance in one direction, in this case for example when container 200 is encroaching on blade 408, but flexibility in the other direction, which may be needed for rolling container 200 up. In the embodiment shown in FIGS. 5A-C, stiffening member 252a takes on a curved profile with a radius of curvature R that may be chosen to optimize its radial stiffness and resistance to lateral bending or rolling up against the radius while being relatively flexible in the opposite direction. FIG. 5B shows a stiffening member 252a of this type rolled up in that opposite direction. This type of stiffener 252a is similar to a bi-stable spring. FIG. 5C shows an embodiment of container 200 that can be rolled up or collapsed in an orderly fashion along a longitudinal central axis of the container in the direction of arrow A in connection with one or more stiffeners 252a.

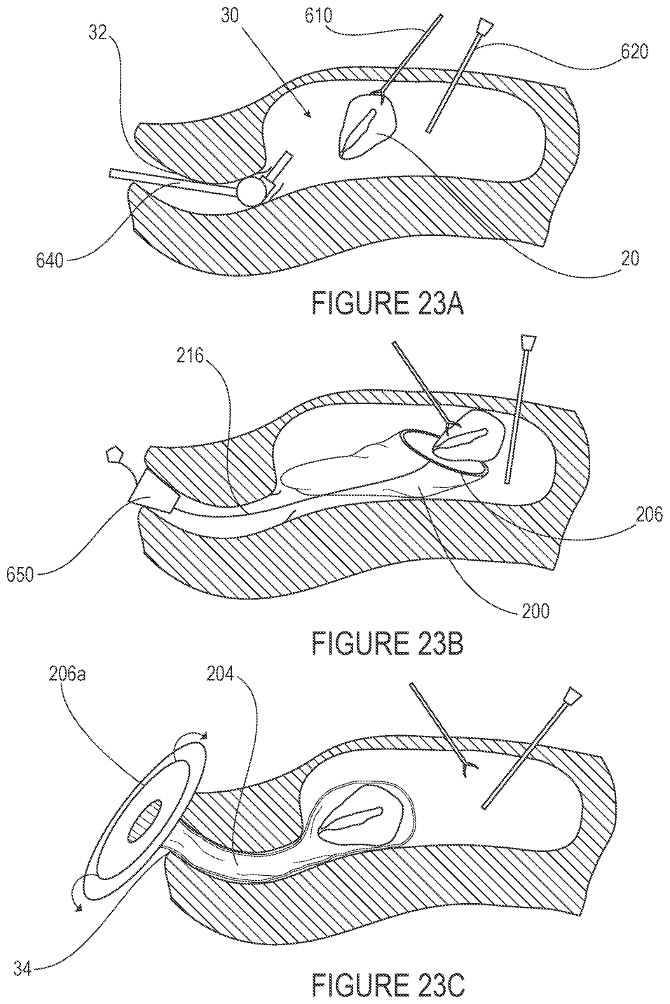

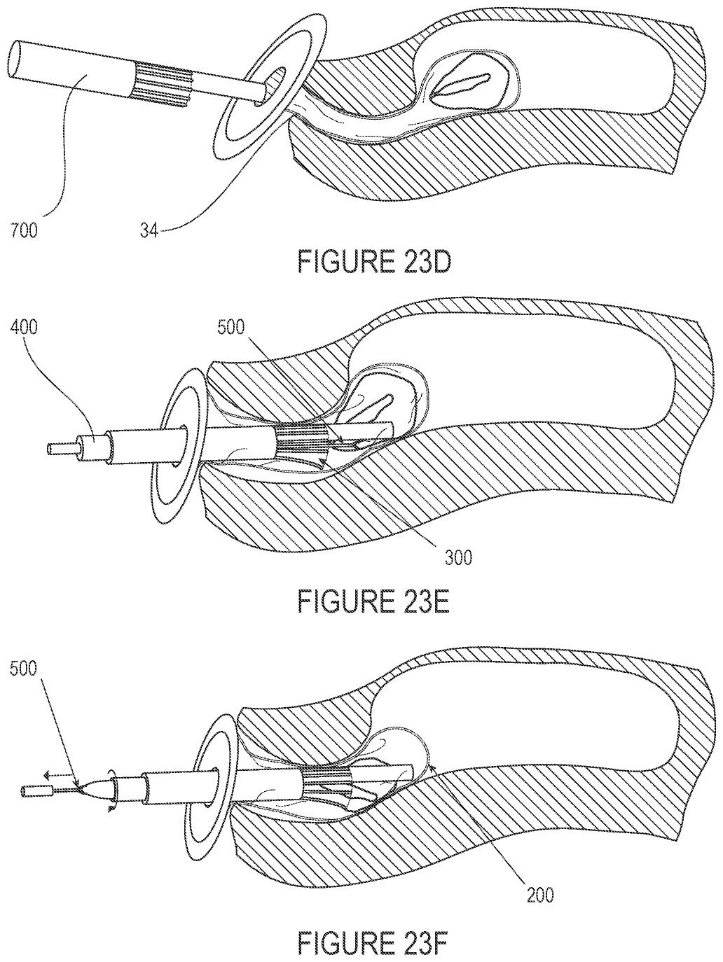

In some embodiments and methods, such as, e.g., the method disclosed in connection with FIGS. 23, 24 and 25 and container 200 embodiments of, e.g., FIGS. 3, 4 and 5, container 200 can be designed intentionally to remain open such that opening 206 and attendant edge 206a are disposed outside the patient's body 31—through opening or port 22 (e.g., surgical port) or natural opening (e.g., vagina 32 and vaginal opening 34). Hence, for the tissue specimen 20 cutting or morcellation step, cutter 400 may be inserted into bag interior 204 through container opening 206 as it is disposed outside body opening 22. Such embodiments and methods therefore do not require that container 200 be closed during the procedure, as specimen 20 is cut/morcellated within bag interior 204 while the opening is under the physician or other operator's control outside body opening 22 so to prevent tissue specimen 20 and other tissue and/or bodily fluids from being in contact with the patient's body 31 during the cutting/morcellation step.

In other methods contemplated herein, however, systems of the present disclosure include components that may be used to deploy container 200 in the body cavity, capture the tissue specimen 20 therewithin, closing an opening 206 of container 206 to enclose the tissue specimen 20 and other tissues and/or bodily fluids within container interior 204, and then morcellating or cutting the specimen 20 within container interior 204. In such methods and embodiments, cutter 400 has been inserted by the physician or other operator into container interior as part of the methods described herein, or cutter 400 may be assembled or manufactured into or is part of a separate container opening 206 as will be described in greater detail below. In addition to cutter 400, other components together with cutter, singly or in combination, such as guard 300, grasper or tenaculum 500 and/or cannula 700 (or cannula-guard embodiments 740, 770) may be used via such a separate container opening for such multi-opening container embodiments. Such other components, singly or in combination, may be made integral to the container 200, with or without cutter 400, to form an “all in one” type of system 100, or may be separate components introduced by the physician or other user into container 200 via this separate opening to accomplish the methods disclosed herein.

It is understood and within the scope of the present disclosure, therefore, that various container embodiments can have only one opening (e.g., container embodiments of FIGS. 3, 4 and 5) or can have two or more openings. In embodiments of container 200 having more than one opening, one or more first openings 206 may be designed to be closed by a physician or other user (via, e.g., any number of mechanisms 210 such as, e.g., a zipper 212, drawstring 218, etc.), to enclose tissue specimen 20 that has been placed within container interior 204, and a separate additional opening 206 may be present through which cutter 400 and/or any number of additional components may be introduced or preassembled as described above. Reference numeral “206” is used herein to refer to any of such container openings, thus allowing it to be understood from the context of the description for a particular container embodiment or method of use as to which type of opening or openings of container is being discussed.

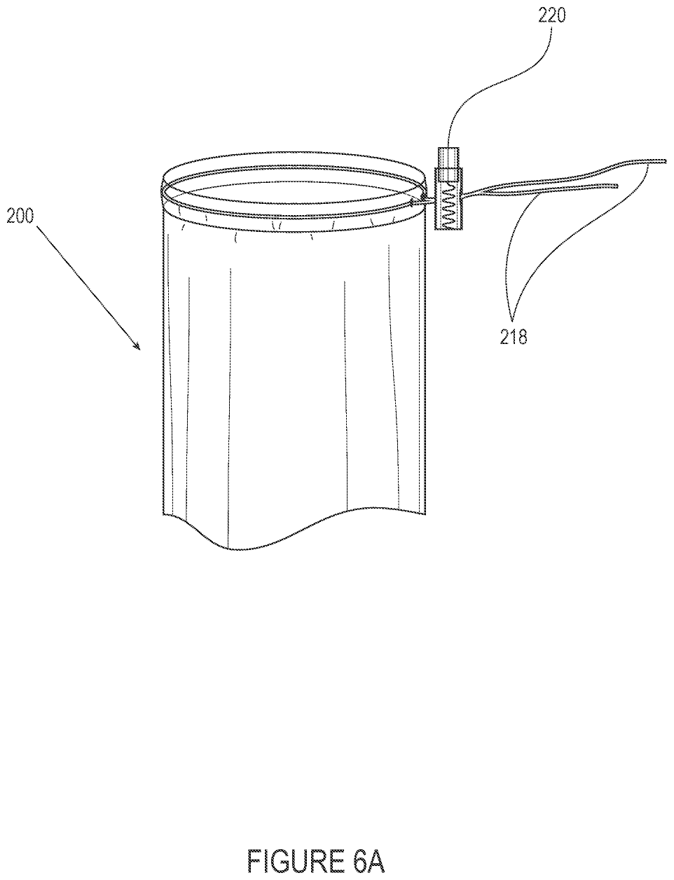

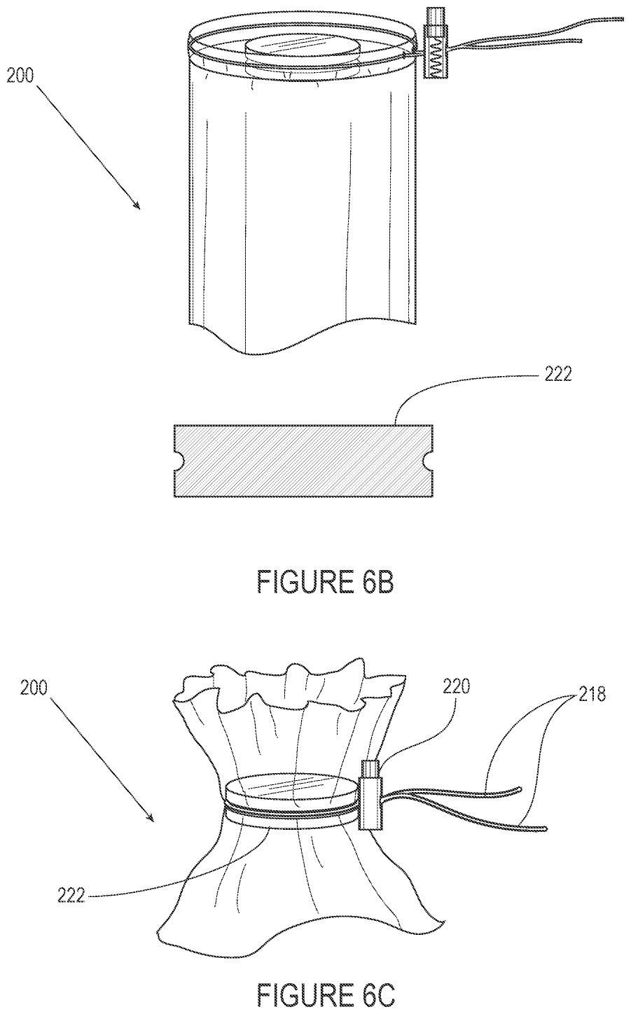

FIGS. 6-14 depict embodiments of container 200 that, as with other embodiments, include elements or features useful in permitting the bag 200 to play an effective role in securely capturing and removing tissue in connection with the systems of the present disclosure. All of the features and designs for enclosure 200 as described herein, and in particular those described in FIGS. 3-14, may be utilized singly or in any combination to suit the performance requirements of the present disclosure. As such, the depiction of a particular embodiment is not meant to be limiting but rather to show possible features and elements associated with and included in this disclosure.

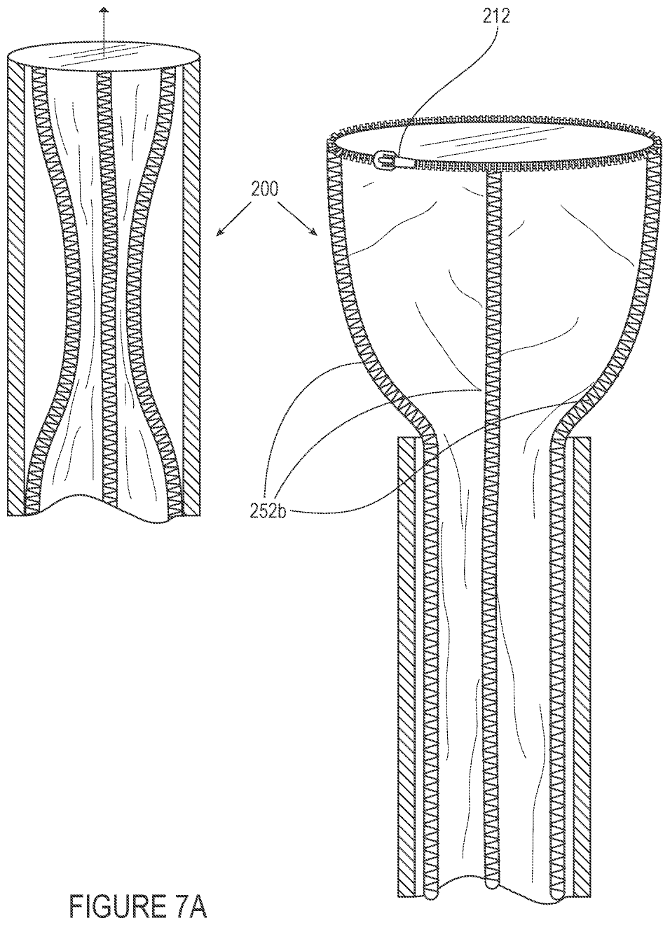

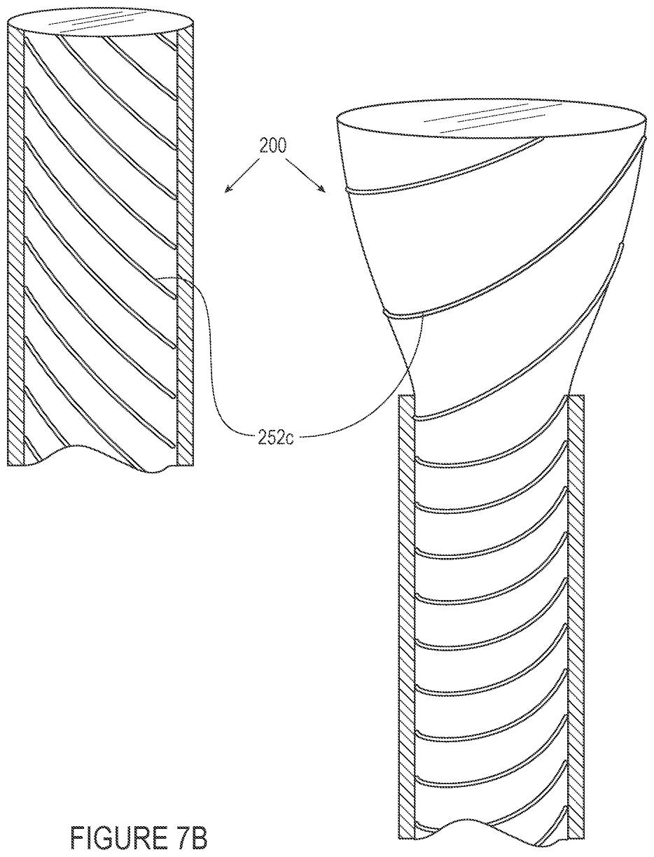

FIGS. 6A-C depict an embodiment of enclosure 200 having features that provide mechanisms for closing bag opening 206. During methods of using the system embodiments of the present disclosure, once a tissue specimen 20 has been placed into the container interior 204, it is desirable to remove the specimen from the patient's body 31 in a way that minimizes the risk that the specimen (and attendant bodily fluids or other tissue) makes unwanted contact with the patient's body tissue and/or bodily fluids, during cutting or morcellation, particularly if it is suspected that cancer or pre-cancerous cells may be present in container 200. FIGS. 7A-B depict additional embodiments of container 200 including features designed to aid container 200 in opening to a configuration that facilitates the placement of a tissue specimen 20 into the bag interior 204 and allow container 200 to move into and maintain a specific three-dimensional configuration or shape. FIG. 7A shows a container 200 in which a spring consisting of reinforcing members 252, here in the form of straight members 252b that extend radially outward relative to a central longitudinal axis of container 200 once removed by the constraint imposed by, e.g., the body port or opening 22 or introducing sheath or cannula 650. Alternatively, the FIG. 7B embodiment includes a reinforcing member 252 in the form of a helically-shaped expansion spring 252c that is curved such that container 200 expansion once unconstrained is comparatively linear, e.g., along the spring's virtual/expansion axis, which such axis can generally be aligned with the central longitudinal axis of container 200. Once the target tissue 20 is within the bag interior 204, the container of FIGS. 7A and 7B can be closed via any suitable closing mechanism. The rigid or spring loaded container embodiment of FIG. 7B affords container 200 additional protection to help prevent container 200 from making contact with the morcellator blade 408 during the morcellation phase. Spring 252c also helps give container 200 a more open shape which facilitates placement of tissue such as specimen 20 therein.

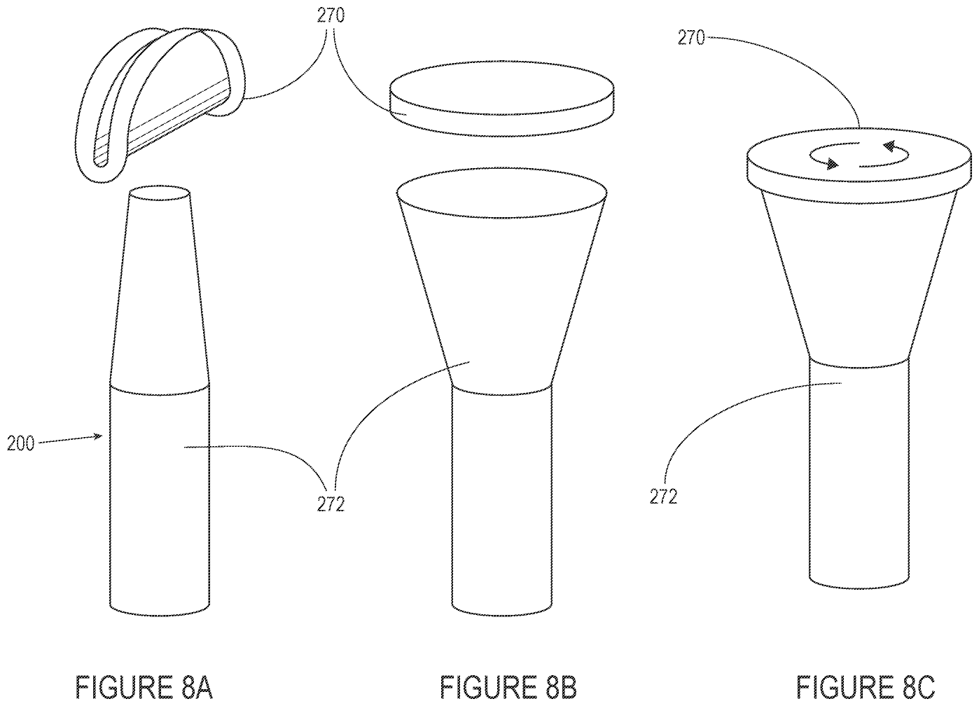

FIGS. 8A-C are directed to a two-part container 200 embodiment in which a first container component 270 may be connected to a second container component 272 in order to securely close opening 206 and keep the contents, such as tissue 20, within the container interior 204. First and second components 270, 272 may be mated/secured to one another in any number of ways, including but not limited to radial loading until a spring engages a lock, or the container can be attached through a threaded or quarter turn screw mechanism. FIG. 8A shows an embodiment of enclosure 200 in a loading position in which first component 270 is folded into a configuration for delivery through a surgical or natural port and a portion of second component 272 is compressed into a frustum shape. In FIG. 8B, both components 270, 272 self-expand or are expanded by manipulation to a larger size. Ideally, a tissue specimen 20 is then placed into the second component 272 after container opening 206 has expanded. Expansion of components 270, 272 may be accomplished by any number of suitable mechanisms, such as by the inclusion of flexible elements within the components or attached to components 270, 272 that cause them to expand (e.g., spring steel or NiTi filaments or parts) or as homogeneous construction in which the material comprising components 270, 272 themselves gives them an inherent ability to expand from a collapsed configuration. Then, as shown in FIG. 8C, first and second components may be securely coupled; in this example by a counterclockwise twisting motion of first component 270 relative to second component 272 as shown by the arrows such that interlocking threads or similar mechanisms engage and effect coupling of the two components. Coupling of first and second components 270, 272 may also be accomplished by the use of tabs, hook and loop closures, zippers, magnets, tongue and groove seals, or any combination thereof to form a secure and ideally fluid-tight connection. During use, second component 272 could be inserted through a surgical or natural port, or it can be designed as part of the morcellation device 400.

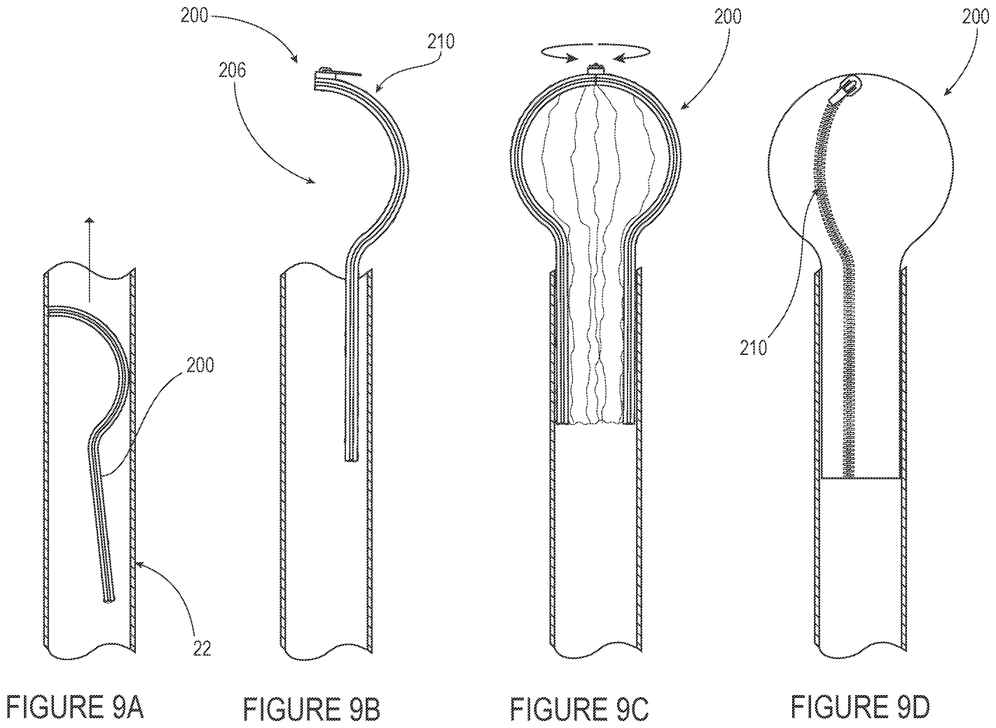

In another embodiment, container 200 is created through a smaller package which can encircle the specimen as shown as it sequentially unfolds. FIGS. 9A-D depict an embodiment of container 200 that takes on a spherical or semi-spherical shape, not unlike that of a lightbulb or plant bulb, when deployed. In this embodiment, enclosure 200 may take on a relatively straight configuration when collapsed for delivery through, e.g., a surgical or natural body port 22 in connection with the present disclosure. This is depicted in FIG. 9A. Once a distal portion 255 of container 200 extends as shown in FIG. 9B beyond a distal end of a surgical or natural body port, one or more wires (not shown) in proximity to the container opening 206 can be manipulated radially to open distal portion 255 of container and create an interior 204 into which a tissue specimen 20 may be placed. The material of container 200 may be made of a flexible but durable material or materials as described elsewhere herein so that when the wire or wires are moved as described, the lightbulb shape of container 200 forms as the specimen 20 is captured within its interior 204. Complete motion of the wire or wires around a full 360 degree or approximate 360 degree path (as shown in FIG. 9C) will allow the edge 206a of container 200 to mate with itself as seen in FIG. 9C. At this point an operator may use any number of closure mechanisms or features 210, such as a zipper 212, to close container 200 such that the tissue sample 20 and other contents are secured therein. Any suitable mechanism, such as a pusher rod or other closure member, may be used to close zipper 212. FIG. 9D shows this embodiment of container in the expanded and closed configuration. Another embodiment of container 200 contemplates zipper 212 moving the opposite direction to close container opening 200, effectively entrapping tissue specimen 200 in container interior 204. A cam mechanism may also be used to rotate the two spring/wire forming shapes between about 1 and about 360 degrees. Other embodiments of a container (not shown) may include an accordion-shaped bag 200 that may be unfurled up to about 360 degrees once in the body cavity 30 and around tissue specimen 20, thus capturing it.

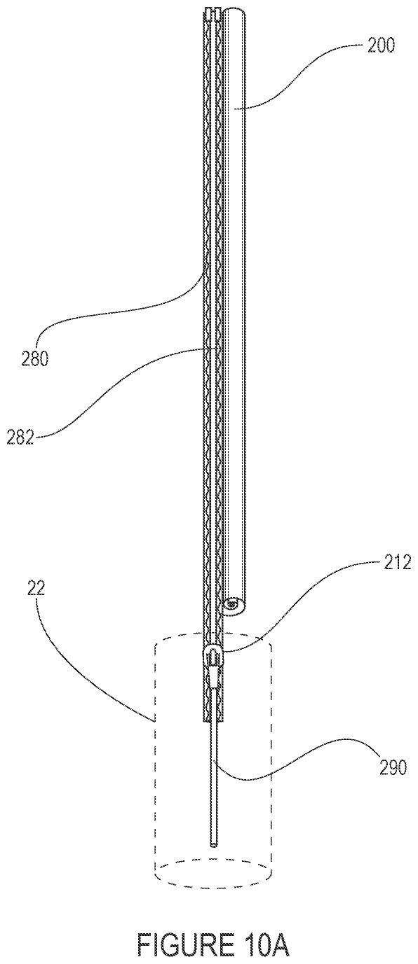

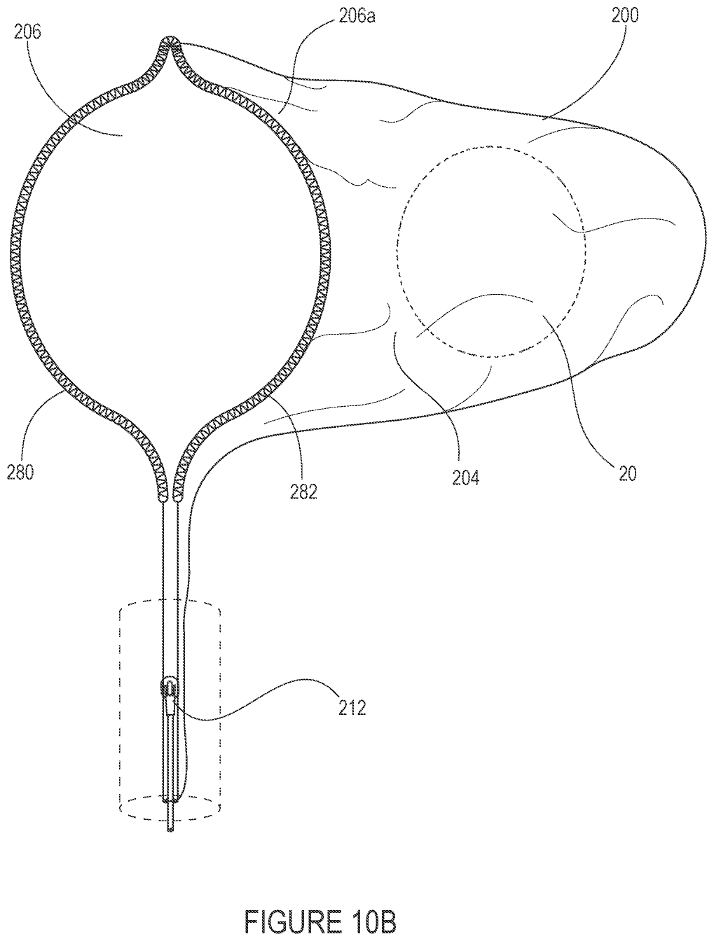

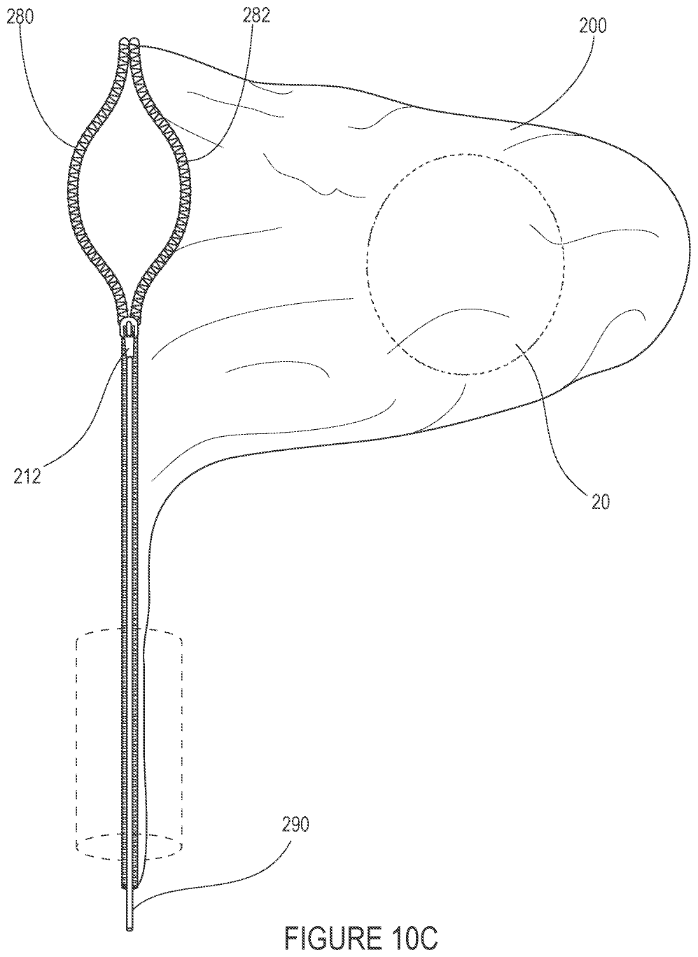

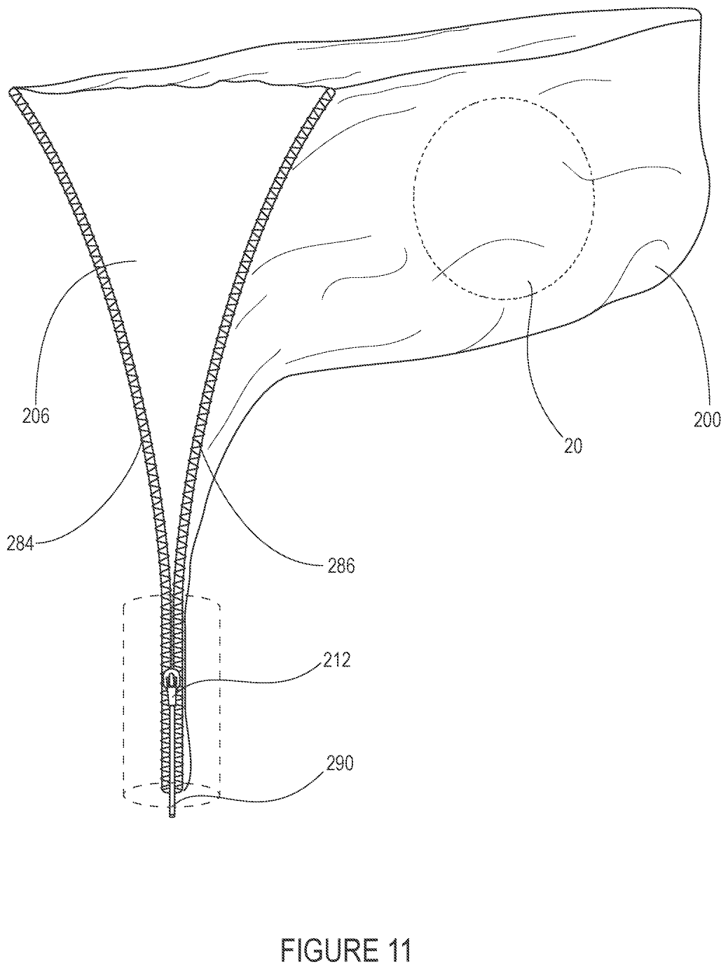

Turning to FIGS. 10A-C, an embodiment of container 200 is shown in operation from deployment, capture of tissue specimen 20, and closure. This embodiment, along with others in which a closure mechanism is described for opening 206 and in which more than one opening is present, is useful for container closure in vivo—after the tissue specimen has been placed therein but prior to specimen 20 cutting or morcellation. For purposes of illustration, the schematic methods and container embodiments of FIGS. 10 and 11 show only one opening 206 through which a tissue specimen 20 moves as a physician or other user places specimen 20 within container interior 204. As described elsewhere herein, for container embodiments in which an opening 206 may be closed or sealed (e.g., the embodiments of FIG. 6-11) one or more additional openings 206 may be present. In particular, an additional opening 206 may be present in which a cutter or morcellator 400 is disposed, either integrally during manufacturing or assembly (such that container 200 and cutter 400 form an “all in one” container and cutting component) or by the physician or other user during the methods described herein. For example, in the embodiments of FIGS. 10 and 11, such an opening is not shown but may be present near the portion of container 200 shown disposed in body port 22. During use, a cutter or morcellator 400 (not shown), with or without a cannula 700, guard 300 or cannula having integrated protector elements (such as cannula embodiments 740, 770), in any combination, may be disposed within this additional opening in container near body port 22 and advanced into container interior 204 after the container closure mechanism 210 (in the case of FIGS. 10 and 11, zipper 212) is operated to close opening 206. Cutter 400 can then be activated, after performing any desirable tensioning or other steps as described herein, to cut or morcellate specimen 20 and to remove the processed specimen and any other tissue or bodily fluid contained in bag 200 out of the additional opening.

It is specifically within the scope of the present disclosure to employ methods using containers having one opening where that opening is not closed but rather moved outside the body port 22 and through which morcellator 400 may be deployed for tissue specimen 20 cutting and removal. It is also specifically within the scope of the present disclosure to employ methods using containers having more than one opening, which are contemplated for the containers shown in the examples of FIGS. 6-11 having closure mechanisms for one of the container openings, in which tissue may be cut/morcellated and removed through an additional opening. Variations of both methods and mechanisms/components to accomplish tissue capture and removal using containers with one or more openings are also within the scope of the present disclosure.

FIG. 10A shows this embodiment of container 200 in its compact form that can be inserted through a port 22 (such as one created by surgical incision or a natural opening such as a vagina, rectum, esophagus, etc.). Two pre-tensioned spring elements 280, 282 may be associated with container opening 206 as seen in FIG. 10B. These elements can be made of any medical grade material having physical characteristics sufficient to actively force container 200 open when deployed, to aid a user in opening container 200, or to at least not materially interfere when a user is opening container 200 to capture a tissue specimen as shown in FIG. 10B. This may be accomplished by way of, e.g., elements 280, 282 being comprised of a shape memory material or by the use of a removable sheath (not shown) that constrains elements 280, 282 until such time that the physician determines the enclosure 200 is to be opened—typically once it is in the body cavity 30. Note that at distal ends 280a, 282a, elements 280 and 282, respectively, may be joined together or otherwise integrated to form a single element so that the entire container edge 206a effectively is defined by elements 280, 282.

As shown in FIG. 10B, the opened bag 200 may take the shape of, e.g., a non-porous fluid-impermeable fish net, which can be manipulated on its proximal end 254 by the physician or surgeon for capturing tissue specimen 20 into the bag's interior 204. Container opening 206 is characterized by a generally oval or circular shape defined by edge 206a, a shape driven at least in part by the configuration of elements 280, 282 when deployed.

FIG. 10C shows the container opening 206 being closed with specimen 20 inside the container interior 204. This may be accomplished through the use of a stiff rod 290, which is attached at one end to a zipper mechanism 212, that can be pushed by a physician through the body port, or natural opening, and possibly through a cannula lumen if desired, to draw opposing sides of bag edge 206a together, forcing elements 280 and 282 to straighten as the container closes 200. Other mechanisms to close and seal container 200 as described herein are contemplated, including the use of a sliding sheath to mate the sides of container edge 206a and seal the opening 206 via, e.g., a sealable bag design. Once the container 200 is sealed, tissue specimen 20 can then be morcellated, through another predetermined opening 206 (not shown) in the bag 200 through which the cutter 400 is fit or disposed, or otherwise processed or manipulated; the device can then be extracted from the patient's body 31. In an all in one concept (not pictured), the container 200 can be physically attached to the morcellation device 400 through any suitable means or processes, such as, e.g., a co-molding process. Container 200 can include a hard retainer at this additional opening (not shown) that is sealed and attached to the tissue cutter.

FIG. 11 shows an embodiment of container 200 in which the opening 206 takes on a more triangular shape defined by edge 206a. As with the FIG. 10 embodiment, in its compact form the FIG. 11 embodiment can be inserted through a channel, a port 22 (such as one created by surgical incision) or natural opening (e.g., vagina, rectum, esophagus, etc.). Two pre-tensioned spring elements 284, 286 may be associated with container opening 206. These elements can be made of any medical grade material having physical characteristics sufficient to actively force container 200 open when deployed, to aid a user in opening container 200, or to at least not materially interfere when a user is opening container 200 to capture a tissue specimen as shown in FIG. 10B. This may be accomplished by way of, e.g., elements 284, 286 being comprised of a shape memory material or by the use of a removable sheath (not shown) that constrains elements 284, 286 until such time that the physician determines the container 200 is to be opened—typically once it is in the body cavity 30.

In this FIG. 11 embodiment, container opening 206 is characterized by a generally triangular or circular shape defined by edge 206a, a shape driven at least in part by the configuration of elements 284, 286 when deployed and the fact that their distal ends 284a, 286a, respectively, are not connected to one another but rather are connected by a portion of the container 200 material (in contrast to the FIG. 10 embodiment).

During use, the FIG. 11 embodiment may be closed as described above in connection with the FIG. 10 embodiment, once the container 200 is sealed, tissue specimen 20 can then be morcellated, through another predetermined opening 206 (not shown) in the bag 200 through which the cutter 400 is fit or disposed, or otherwise processed or manipulated; the device can then be extracted from the patient's body 31. In an all in one concept (not pictured), the container 200 can be physically attached to the morcellation device 400 through any suitable means or processes, such as, e.g., a co-molding process. Container 200 can include a hard retainer at this additional opening (not shown) that is sealed and attached to the tissue cutter.

Other container embodiments may include features that stiffen the container 200 and/or enable closure of a container opening associated with a closure mechanism 210 through a natural body port (e.g., transvaginally) without the need of assistance from tools deployed through other ports. In some embodiments, enclosure 200 may include stiffening elements and two openings having closure mechanisms 210 (in addition to the additional opening through which a cutter and optionally one or more additional components may be deployed), one or both of which can be closed with the use of a robot and/or instruments from the one or more other ports. Container closure could be accomplished by any number of mechanisms 210 or methods, such as the use of one or more drawstrings, magnets, zipper or any other closure mechanism 210 such as described elsewhere herein. In some embodiments, container closing may be accomplished through the natural body port; e.g., in a gynecological procedure, through a patient's vagina 32, such as when an open end of bag 200 captures the tissue specimen 20 (e.g., uterus) and one or more stiffening members in container provides the structure necessary to enable a physician or other user to employ a zipper, pulley or other mechanism associated with container 200 to close it. In other embodiments (not shown), container 200 may include a flap that may be closed by a physician or other user via a mechanism such as, e.g., a zipper or drawstring after tissue specimen 20 is captured within container interior 204, thus sealing container 200. In such an embodiment, container 200 may be closed by robotic and/or other instruments via one or more surgically-created ports other than a natural body port, such as, e.g., abdominal ports. In other embodiments, container 200 may include one or more stiffening members and a zipper or other closure mechanism 210 that closes bag 200 in a direction from the body's natural port (e.g., vagina) towards the distal end of the container or bag 200. This embodiment affords a way for the one or more stiffening members to be used to close container 200.

Other embodiments of container 200 may include closure mechanisms 210 involving one or more magnets, such as those that may be automatically triggered via a linkage of multiple magnets. Such a linkage can allow the container opening 206 to open wide enough to permit entrance of tissue specimen 20 therethrough and into container interior 204, but then the linkage can close the container and seal the tissue and liquids inside. For example, container 200 may include a series of magnetic strips along the bag's length that may be activated mechanically, magnetically, electromagnetically or otherwise to join together in a hinged relationship by virtue of magnetic force to close enclosure 200 after specimen 20 has been placed therein. Additional embodiments contemplated herein include the use of a magnetic closing mechanism in which adjoining portions of container edge 206a are lined with one or more magnets such that magnets of opposite polarity mate to join portions of the edge 206a together to close container opening 206. In one configuration, zippers as shown in the various figures and embodiments herein may be replaced by or supplemented with magnets to effect adequate and reliable, and perhaps more automatic, container closure.

Yet further mechanisms for closing container opening 206 as contemplated by the present disclosure include those in which the container distal end 255 near opening 206 (or any portion of container near an opening 206) may be rolled or folded upon itself such that the edge 206a mates to close opening 206, perhaps supplemented by a clip, hook and loop-type fastener, latching mechanism or the like on or associated with bag surface 202 to secure the folded or rolled portion of container into place.

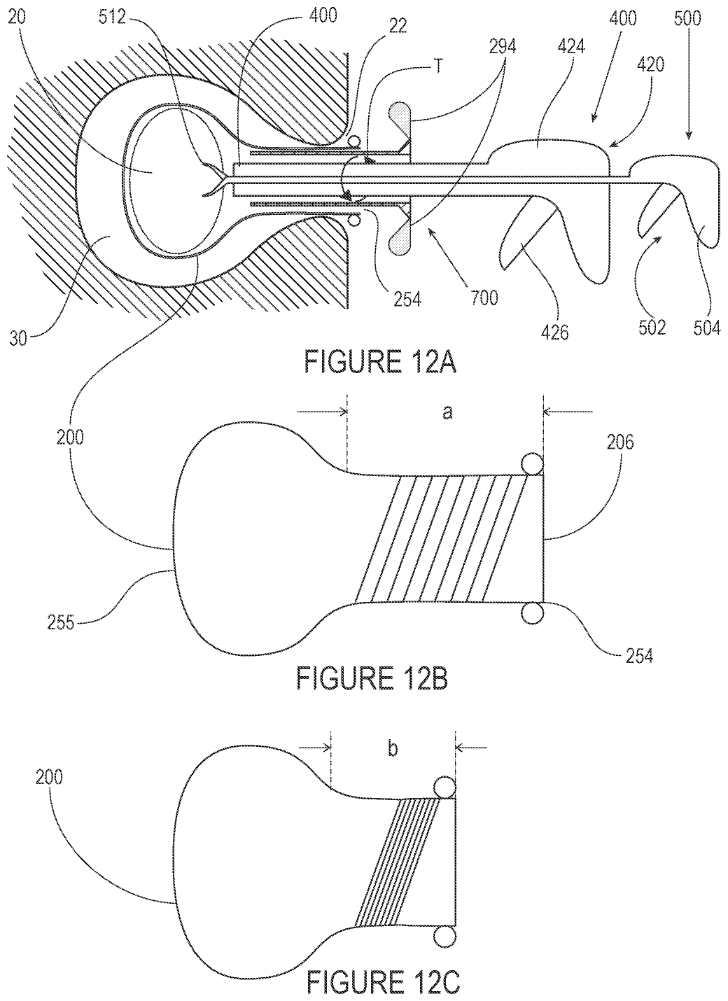

As described herein, it may be useful to place axial tension on container 200 after the tissue specimen 20 is captured but prior to and/or during all or a portion of the tissue morcellation process. FIGS. 12-14 depict embodiments of the present disclosure in which tensioning of container 200 during use may variously be accomplished.

FIGS. 12A-C illustrate an embodiment of container 200 and its use in vivo with components of a system of the present disclosure where a portion of container 200 may be twisted, with or without the use of a handle 292, as a way to apply tension to container 200, thereby shortening its axial length and facilitate efficient and accurate processing of tissue specimen 20.

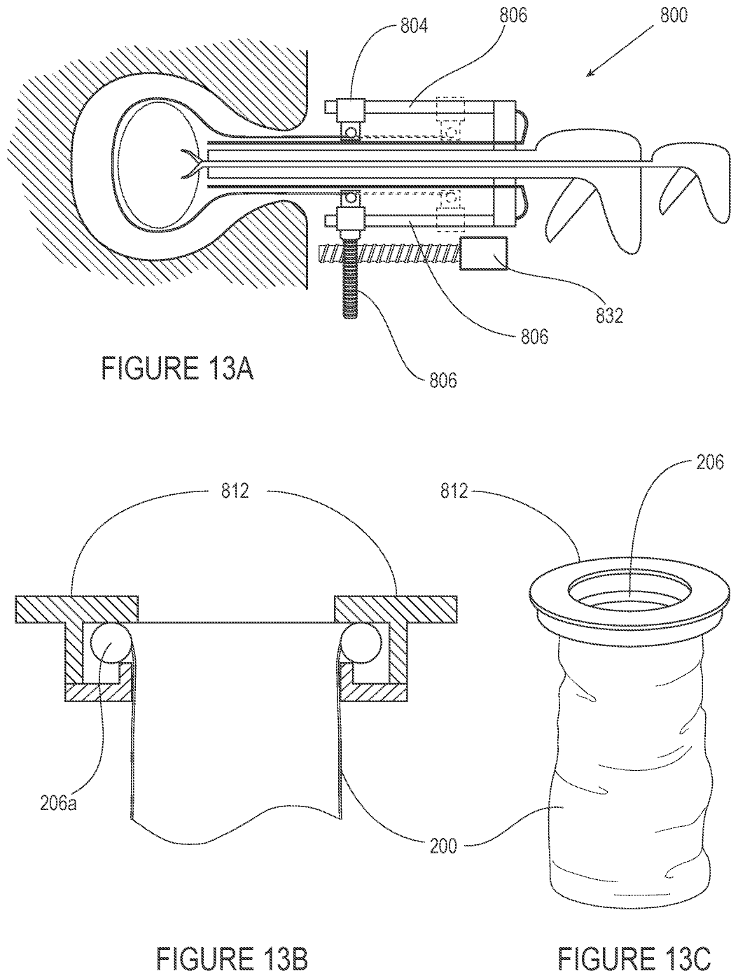

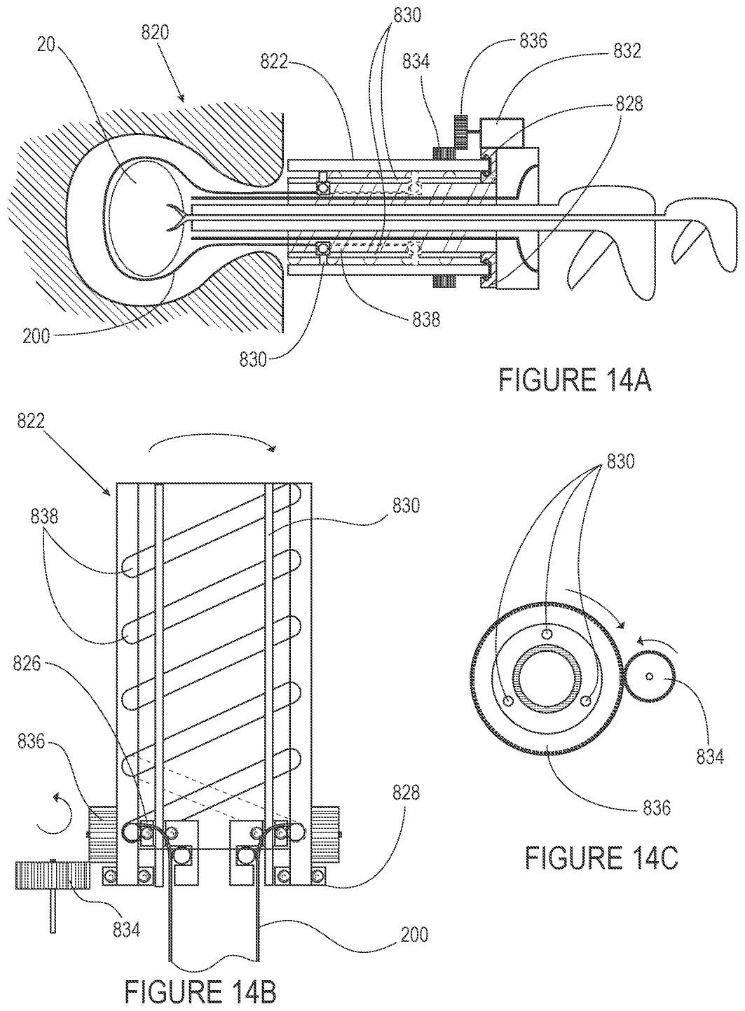

In the schematic of FIG. 12A, container 200 is shown deployed in a patient working cavity 30, having within its interior 204 a captured tissue specimen 20. A tissue grasper 500 is shown in apposition with the specimen 20 and extends out of a distal end 420 of cutter/morcellator 400. A proximal portion 502 of the tissue grasper terminates in a handle 504 that includes a trigger 506 for activating the tissue grasper distal portion 510. Likewise, a proximal portion 422 of cutter/morcellator terminates in a handle 424 that includes a trigger 426 for activating the cutter blade 408. A cannula 700 around which container 200 is placed, having a central lumen 702 through which are disposed cutter 400 and tissue grasper 500 is also shown in the patient's access opening 22 and at least partially into working cavity 30. A handle 254 is disposed at the proximal end 254 of container 200 in the vicinity of patient access opening 22. Handle 254 may take on the configuration as shown in FIG. 12, or it may be larger or have additional features to facilitate ease of use. Handle 254 may be attached onto or be an integral part of container 200; alternatively, handle 254 may be added onto the bag 200 during the procedure, after it is extracted from the patient port (e.g., vagina, etc.) and prior to the bag shortening step. Handle 254 may be operated manually or by any number of automated mechanisms, such as by the use of a motor used in connection with a geared transmission system or the like as described, e.g., below in connection with the embodiments of FIGS. 13 and 14.

In use, a physician or other operator applies tension on container 200 by twisting it, in one embodiment by use of handle 254, in the direction T indicated in FIG. 12A. This act of twisting shortens the bag 200, thereby putting tissue specimen 20 into contact with the container distal end 255 as shown in FIG. 12A. Together with the optional application of force on specimen 20 and/or by gripping at least a portion of specimen 20 with grasper 500, continued twisting brings tissue specimen 20 up against the morcellation blade 408 to facilitate the beginning of the process of cutting the tissue specimen 20. As the tissue specimen 20 is processed by the cutter, continued twisting of bag 200 and optional application of axial force via grasper 500 will facilitate a reliable and efficient cutting effort as enclosure 200 continues to shorten and the specimen 20 is reduced in size as it is processed by the cutting action of blade 408.

FIGS. 12B and 12C illustrate how container 200 may be shortened, depicting an initial linear dimension ‘a’ of a portion of enclosure 200 that is transformed to a shorter dimension ‘b’ by virtue of this twisting action. The difference between dimension ‘a’ and dimension ‘13’ (a-b) may vary, depending on the application for which the system is being used. In some instances, such as a hysterectomy, this difference can be approximately 20.0 centimeters, but could be as much as about 50.0 cm or more or as little as 1.0 cm or less.

FIGS. 13A-C depict another embodiment in which enclosure 200 may be tensioned by an automated telescoping mechanism 800 to effect efficient processing of the tissue specimen 20 as described above. Here, container 200, tissue grasper 500, cutter/morcellation device 400 and their various features and components are shown in vivo in FIG. 13A in similar fashion to FIG. 12A. Tissue specimen 20 is disposed in container interior 204.

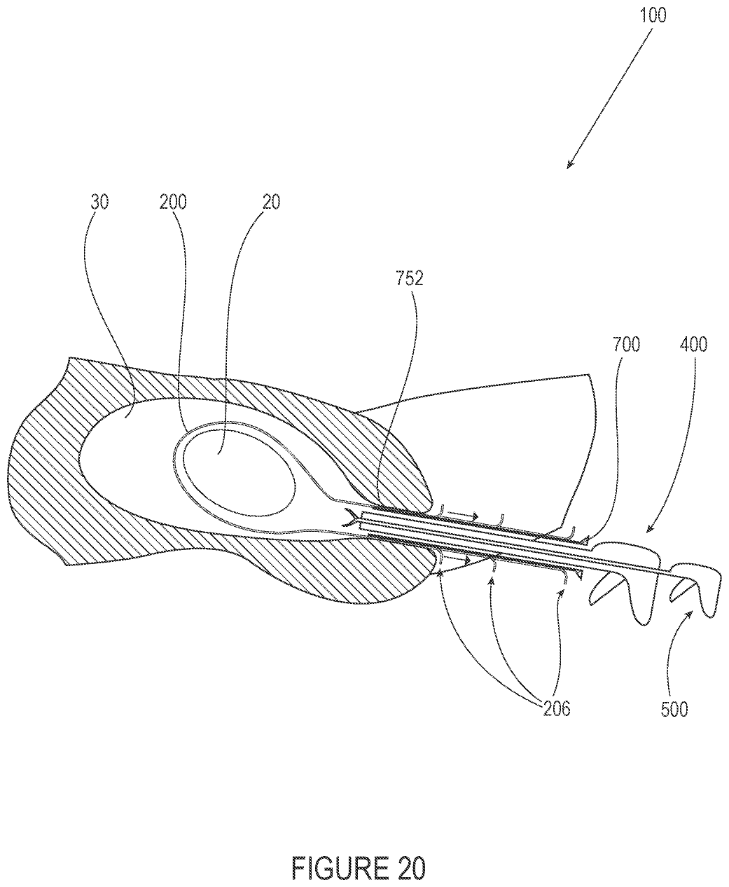

FIGS. 13A-C show a retention piece or clamshell housing 812 attached to a proximal end 254 of container 200 in the vicinity of container opening 206. Retention piece 812 may be so attached after the opening 206 is in place outside the patient's body 31 at opening 22 as shown in FIG. 13A; alternatively, embodiments may include a container 200 having a retention piece pre-attached to or integral with container 200. Clamshell 812 can include handles (not shown) as described above in connection with the FIG. 12 embodiment. These handles can be used in twisting container 200 as described herein or for simple axial tensioning along a long shaft as described below in connection with FIG. 20.

Retention piece 812 allows for ready manipulation of the system 800 and container 200 to effect the desired tension during the processing of tissue specimen 20. Housing 812 includes bearings 804 that are axially slidable on rails 806 to keep container 200 aligned in a relatively straight configuration as a lead screw or drive gear 834 powered by a motor 832 pulls container 200 out of the patient working cavity 30 and through opening 22 in a linear fashion. By automating the container retraction process, the physician or operator is free to have one hand on the morcellator 400 and the other hand on the tissue grasper/tenaculum 500 as the container is tensioned and retracted automatically by telescoping mechanism 800. A control module 802 (not shown) can be programmed to provide a specific level tension or axial force on container 200 during operation to keep tissue specimen 20 flush with or in apposition against the morcellator blade 408 as described above. Control module 802 can also be programmed or set to pull the entire bag or container through the patient's access opening 22 when the tissue specimen 20 is or has become by virtue of the cutting action of cutter 400 small enough to fit therethrough. This container tensioning system 800 can be attached to a cannula 700 or can be separate from cannula 700. And while telescoping mechanism 800 is shown in FIGS. 13A-C as being motor driven and controlled by a programmable control module 802, mechanism 800 may also be otherwise manually activated and operated by the physician as desired, semi-manual embodiments may be used, such as motors operated and controlled by the physician or other operator, or mechanical (non-electrical and/or non-automated) embodiments operated and controlled by the physician or other operator. In other embodiments, mechanism 800 may operate via a rack and pinion mechanism, either by hand or motorized with a control system to apply a constant or controlled but variable force on container 200 or to operate under velocity control, etc.

FIGS. 14A-C depict another embodiment in which enclosure 200 may be tensioned by linear motion that is achieved through a telescoping or helical cam mechanism 820. Here, container 200, tissue grasper 500, cutter/morcellation device 400 and their various features and components are shown in vivo in FIG. 14A in similar fashion to FIGS. 12A and 13A. Tissue specimen 20 is disposed in an interior 204 of bag 200.

Mechanism 820 includes a helical or cylindrical tube 822 as shown in the cross-sectional views of FIGS. 14A-B. Within a central lumen 824 of tube 822, typically on an interior wall is disposed a helical groove, ridge or other feature 838 suitable for receiving or otherwise engaging with a clamshell element 812, not unlike that of the FIG. 13 embodiment. As tube 822 is rotated along a central longitudinal axis, either by a motor 832 as shown in FIGS. 14A-C or by hand or other mechanical means (not shown), container 200 is then drawn linearly down the cylinder lumen 824 by the cooperation of clamshell element 812 and cooperating bearings 804 in the groove or ridge feature 838. Alignment shafts or guide rails 830 and associated bearings help keep proximal end 254 of container 200 in proper alignment and to prevent rotation of enclosure 200. An optional bearing 828 disposed at a distal end 825 of helical tube 822 enables mechanism 820 to spin with respect to the various components of the present disclosure (e.g., container 200, cutter 400, grasper 500, cannula 700, etc.). Between 1 and 4 or more shafts may be used.

A drive gear 834 and a driven gear 836 are shown in FIGS. 14A-C, and in cross section along with rails 830 in FIG. 14C. A motor 832 powers rotational movement of drive gear 834 to which it is connected, which in turn effects rotational movement of driven gear 836 that rotates tube 822.

In an alternative configuration (not shown), helical cam mechanism 820 may be disposed within an interior 204 of container 200 and operate as described herein. In such a configuration, container 200 may be drawn linearly around the outside of tube 822 by the cooperation of clamshell element 812 and cooperating bearings 804 in groove or ridge feature 838 that is located on an exterior surface of tube 822 rather than within a lumen 824 of tube 822.

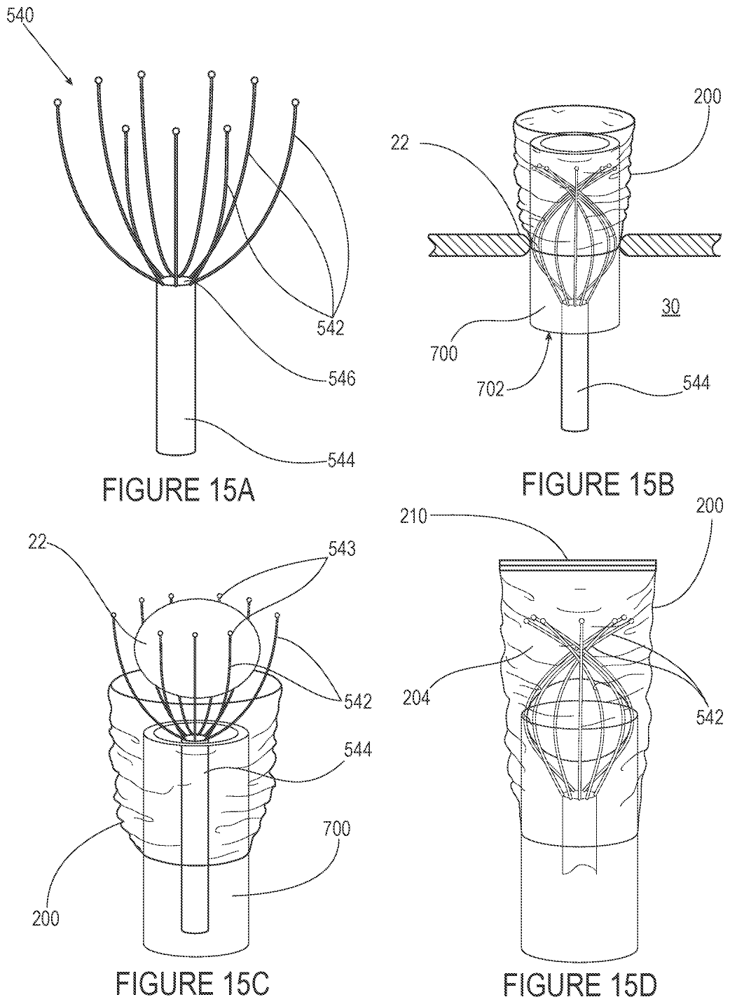

FIGS. 15A-D depict an embodiment of an introducer 544 and a blunt-tined tissue grasper or tenaculum 540 and a method of its use with the systems and components of the present disclosure. In FIG. 15A, grasper 540 is shown as including any number of tines 542 extending from an interior volume or lumen 402 of a cutter or morcellator 400. Tines 542 on their proximal end (not shown) may be connected to or integrated with a pusher or other instrument that may be manipulated, with or without a handle and/or trigger mechanism to move tines 542 of grasper 540 through cutter lumen 402 and into an interior 546 of introducer 544 as shown in FIGS. 15B-D.

Tines 542 may be made from any suitable biocompatible material, and in particular can include plastic or metallic materials that exhibit spring-like or even shape memory behavior so that they take on a desired configuration (such as that shown in FIG. 15A) when disposed outside of any constraining structure (e.g., cutter 400 and/or introducer 544). The number of tines 542 may be optimized, along with their cross-sectional shapes and how they are configured and arranged relative to one another, to balance the requirements of being able safely and reliably to capture and move a tissue specimen 20, maintain a desired shape when deployed, and be flexible enough to collapse within the any constraining component such as cutter 400 and introducer 544. In the open, fully unconstrained configuration of grasper 540 shown in FIG. 15A, tines 542 extend beyond a distal end 420 of introducer 400, springing to a predetermined “capture” arrangement as would be assumed within a patient working cavity 30 for placement of a tissue specimen within a volume created among the tines as so deployed.

In FIG. 15B, tines 542 are shown extending out from a cutter distal end 420 as discussed with respect to the view of FIG. 15A. However, here, tines 542 have not taken on their fully open or unconstrained shape as they are partially constrained within a central lumen 702 of cannula 700. Here, introducer 544 has been placed within the body of a patient 31, perhaps into a working cavity 30 but in the view of FIG. 15B at least through a patient's access opening 22 for use according to methods of the present disclosure. Container 200, which optionally may be integrated with introducer 544, is shown surrounding distal end 548 of introducer 544 and is within the interior of patient's body 31.

FIG. 15C depicts grasper 540 capturing a tissue specimen 20 after tines 542 have been axially extended beyond introducer distal end 548. In this particular sequence, tissue specimen 20 has been captured from patient cavity 30 and lies within the assembly of tines 542. Specimen 20 and a portion of tines 542 are being moved into an interior of container 200 through container opening 206 and towards the cutter/morcellator distal end 420 on which a blade 408 may be disposed. Optional atraumatic tips 543 may be disposed on an end of one or more tines 542 to prevent or mitigate damage to container 200 or tissue other than tissue specimen 20. In FIG. 15D, the grasper 540 and specimen 20 are now completely contained within the interior 204 of container 200. Via other tools such as may be used via a laparoscopic port or via another closure mechanism 210 as variously described herein, the container opening 206 is now closed and sealed along its edge 206a, safely securing specimen 20 therein. At this point, specimen may be prepared for morcellation as described elsewhere herein, and grasper 540 may be advanced out of the distal end 548 of introducer 544, all within the interior 204 of sealed bag 200, so that the specimen 20 may be released from the tines 542 and the tines may be retracted axially through the lumen 402 of cutter. The physician may then manipulate container 200, cutter 400 and any other instruments (such as a traditional grasper shown elsewhere herein) to pull tissue specimen towards blade 408 for morcellation and removal through the patient's body 31.

One aspect of the systems and method described herein is the concept of a guard or protector element or component 300. Among other benefits, guard 300 helps to prevent container or enclosure 200 from contacting the cutting mechanism 400. Guard 300 also inherently helps to prevent cutter 400 or other components of the present disclosure from damaging or engaging with tissue in the patient's body 31 that is outside the container interior. In a gynecological example, such tissue may include, e.g., the bowel and/or bladder or other tissues that may be in the vicinity of the patient's pelvic cavity in which the system of the present disclosure is deployed. Other tissues in other cavities, such as, the abdominal or thoracic cavities, may also be inherently protected by guard 300 during use. Guard 300 can take several configurations. In one embodiment such as that shown in FIG. 1, guard 300 is a cone-like element taking on a shape of, e.g., a collapsible or partially collapsible funnel that can compress and enter a deployed container interior 204 through a patient port then expand and accommodate the tissue specimen 20 that is to be removed. In FIG. 1, guard 300 is slightly or moderately asymmetric relative to its central axis, such that it is not a right circular conical shape (or frustum thereof) but rather an oblique circular conical shape (or frustum thereof). However, guard 300 may take on a conical shape in the form of a right cylindrical cone or frustum in some embodiments. The asymmetry of guard 300 shown in FIG. 1, forming a frustum of an oblique cone, aids in guiding tissue specimen 20 to a particular portion of cutter blade 408, as does cannula asymmetric extension 776; benefits of this guiding action is discussed below in greater detail.

This embodiment of guard 300 is shaped such that a tensioned bag 200 containing specimen 20 cannot make contact with the cutter 400 and/or tissue grasper/tenaculum 500. Guard 300 possesses adequate hoop strength to prevent it from collapsing, fully or partially, on or near blade 408 while guard 300 is axial tension with container 200. This embodiment of guard 300 also has sufficient stiffness to prevent it from contacting blade 408 when loaded radially. The guard 300 could include ribs of spring steel or shape memory material, such as nickel titanium alloys, which could be coated in silicone, PTFE, ePTFE or other plastic which can be molded around or layered above or below the ribs.

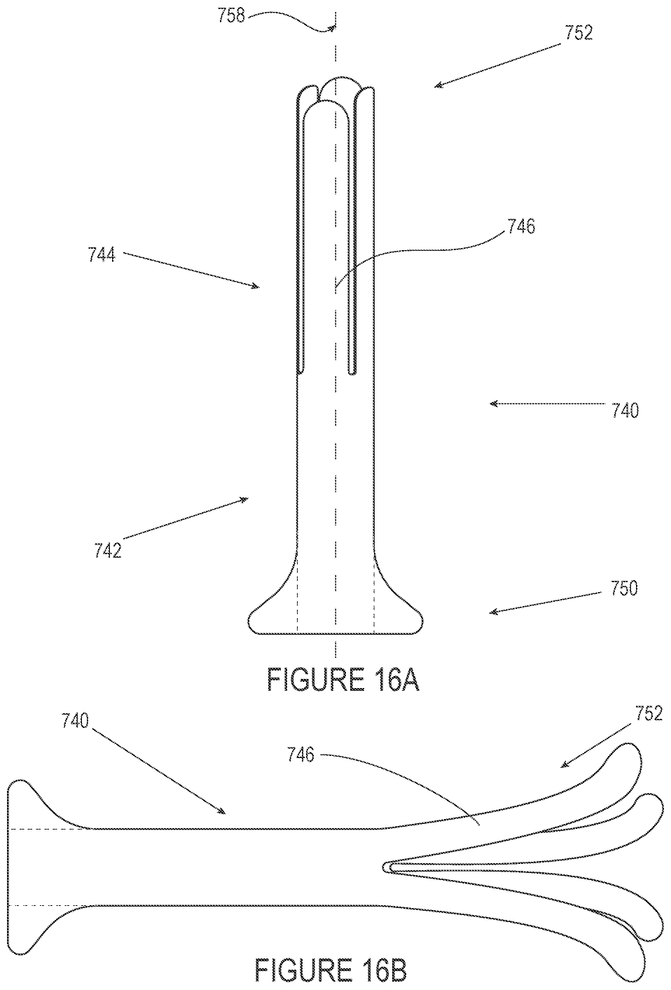

FIGS. 16A-B depict a cannula embodiment 740 having a main portion 742 and a protector portion 744. Protector portion 744, which can function as a guard 300 to protect container from damage during cutting and which can also aid in guiding a tissue specimen 20 towards cutter blade 408, is shown including a number of elements 746. Protector elements 746 in this embodiment are integrated as part of cannula 740. A cannula proximal end 750 may incorporate a flared feature as shown in FIGS. 16A-B for ease of handling by a physician or other operator. Cannula proximal end 750 may also or instead include one or more handles (not shown) for similar ergonomic and operational advantages.

There can be anywhere from one to thirty or more protector elements 746 that are a part of cannula protector portion 744 (four such elements 746 are shown in FIGS. 16A-B). Elements 746 may be attached to cannula main portion 742 or may be of an integral, seamless construction such that cannula 740 is a unitary member. Elements 746 are designed to provide enough stiffness to serve their guarding and guiding functions but to have enough radial flexibility relative to a cannula central axis 758 to bend and take on a variety of configurations. In one construction, protector elements 746 can be spring-loaded. Elements 746 may be made from a shape memory material such as a nickel titanium alloy, a spring steel alloy, a plastic or other flexible metal such that the they can collectively have a small diameter when passing through the patient opening 22 or other port, and open to a larger diameter, either by activation or release from a constraint (or both) once inside the patient's working cavity 30. Elements 746 may include rubber or other relatively soft components on their distal tips to help prevent container damage, particularly from puncture, during the various steps in the methods described herein. In addition, a physician or other operator may use grasper 500 to grip at least a portion of specimen 20 and pull the tissue specimen 20 axially towards the cannula distal end 752 into contact with the protector elements 746 in general alignment with the cannula central axis 758. If the size of specimen 20 is large relative to the diameter of central lumen 702, this action will cause protector elements 746 to bend out radially to accommodate the specimen 20 therewithin. This action combined with the design features that permit elements 746 to bias inward but spring outward radially as described above demonstrates the utility of this embodiment 740 of cannula.

Once positioned as described, tissue specimen 20 may be drawn towards the cutter/morcellation 400, and in particular blade 408, by, e.g., tensioning of container 200 while optionally maintaining axial tension on specimen 20 by grasper 500, etc., as described elsewhere herein. As container 200 is tensioned, protector elements 746 provide a barrier between the morcellation blade 408 and container 200, protecting enclosure 200 from being pierced, torn or otherwise damaged during the cutting process to maintain the integrity of the container and preventing leakage of tissue or bodily fluids into the patient's body 31.

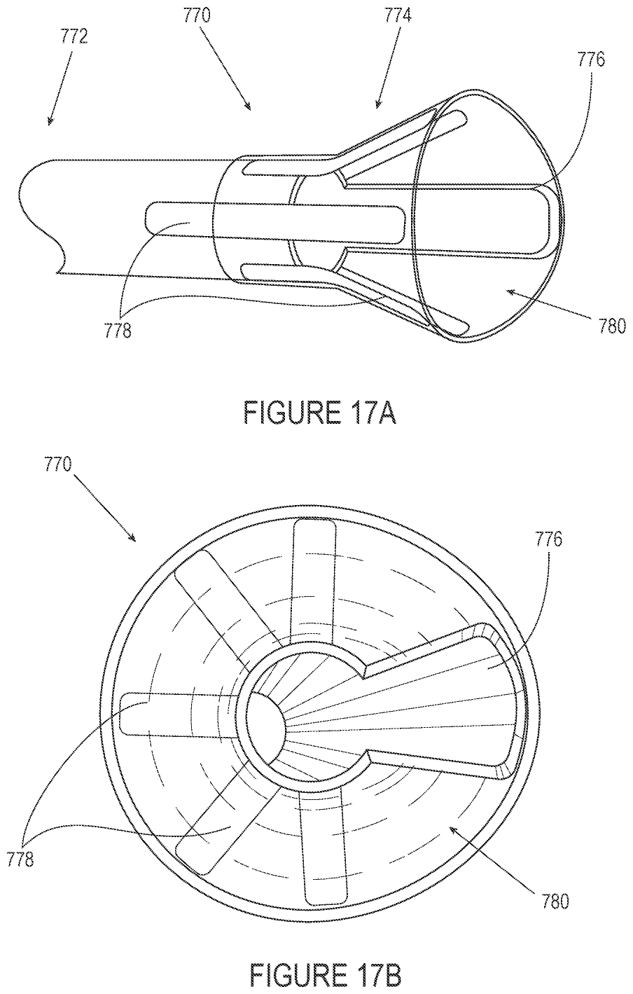

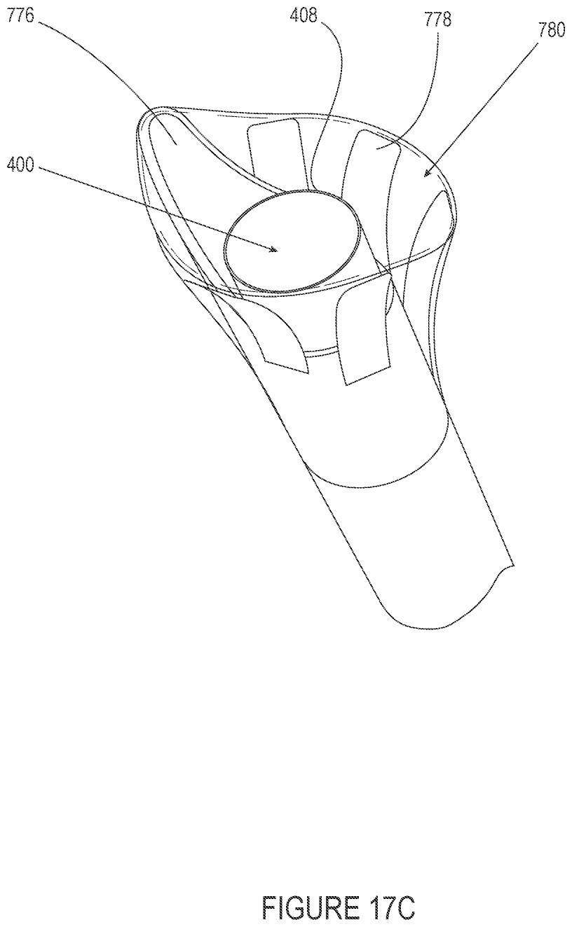

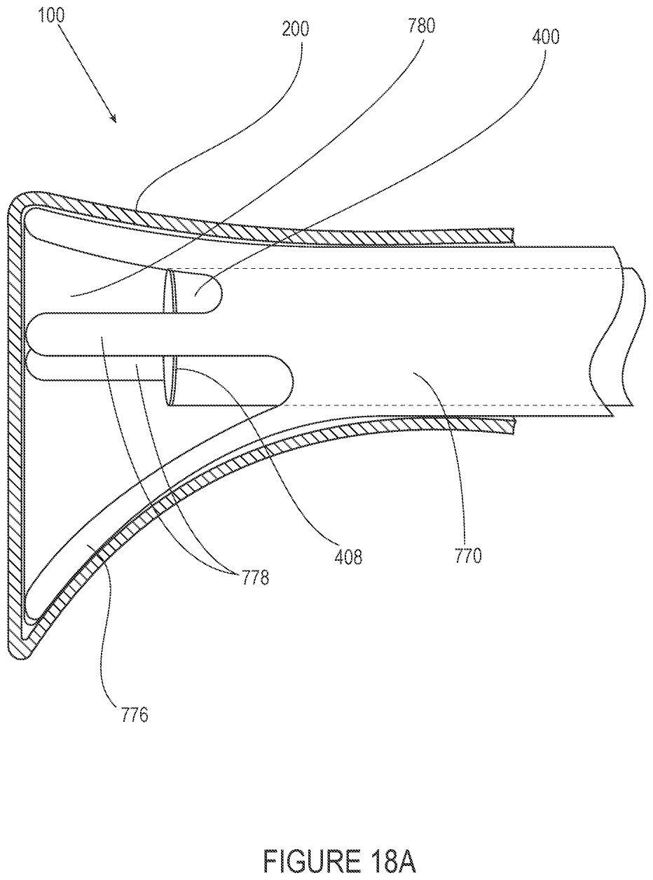

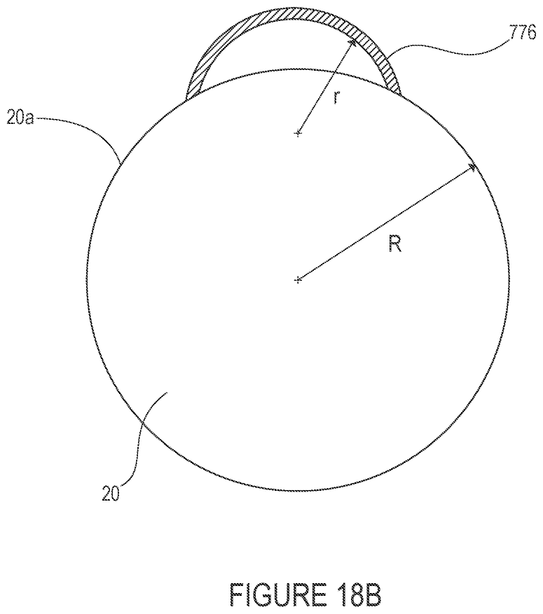

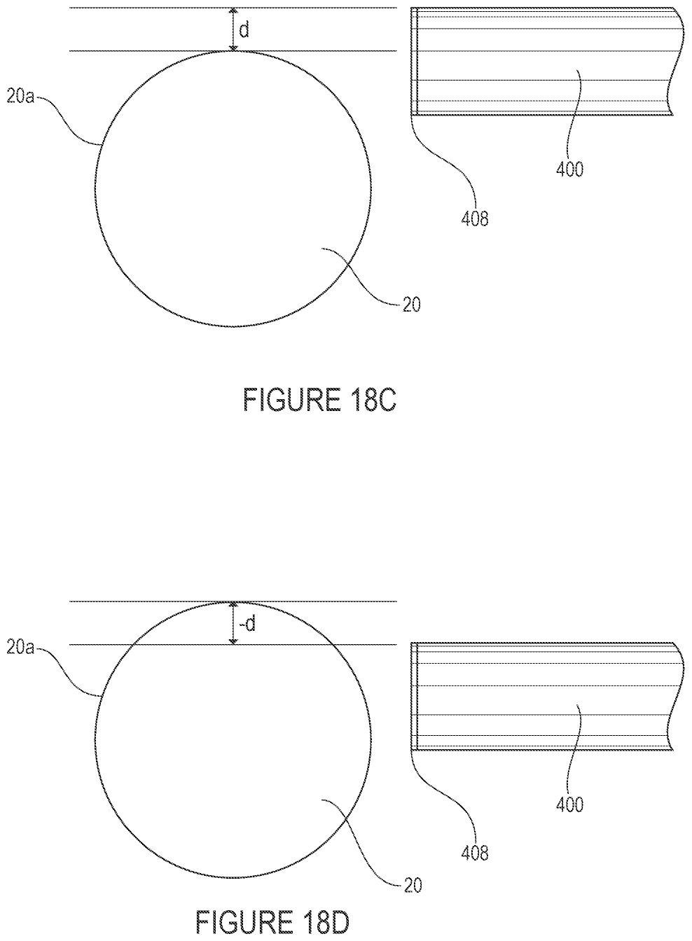

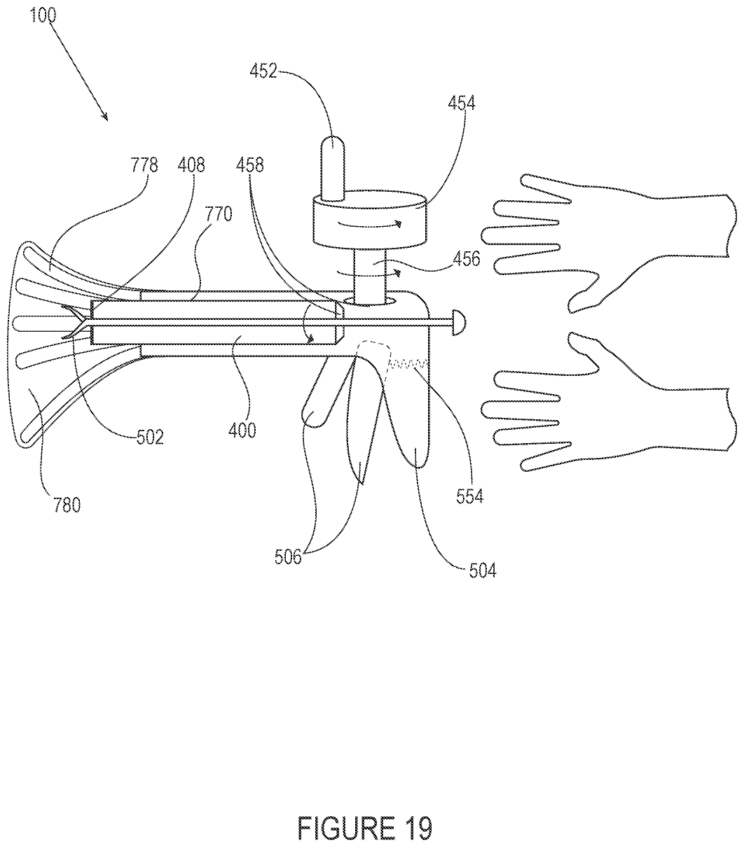

In another embodiment of the present disclosure, FIGS. 17A-C depict various views of cannula 770 integrated with a guard/extension 776, protector elements 778 and an enclosing element 780. FIGS. 18A-D depict cross-sectional (FIG. 18A), schematic end-on (FIG. 18B) and schematic side (FIGS. 18C-D) views of a portion of a system according to the present disclosure that incorporates the cannula 770 of FIGS. 17A-C, while FIG. 19 schematically depicts a system incorporating the elements of FIGS. 17-18 that utilizes a manually powered cutter 400. Turning to FIGS. 17A-C, cannula 770 includes a protector portion 774 adjacent a main portion 772. Integrated with or attached to protector portion 774 is an extension 776, at least one protector element 778, and an enclosing element 780.

During methods of the present disclosure, it is advantageous to protect container 200 from being damaged by the blade 408 of the cutter/morcellator, whether or not that blade 408 is actively being used to process the tissue specimen 20 as described herein. It is also advantageous to guide tissue specimen 20 as it is processed by cutter 400 such that it “feeds” or can be preferentially directed to a portion of the system of the present disclosure such that a designated area of the blade 408 contacts the tissue specimen 20 as that specimen is drawn against the cutter 400 for processing. This embodiment presents design characteristics that facilitate these advantages.

In general, cannula 700, which may be inserted in the patient access opening 22, whether that opening is surgically created or a natural opening (e.g., vagina, rectum, esophagus, etc.), serves as a conduit through which a cutter/morcellator 400 may be inserted and tissue specimen 20 withdrawn according to methods described herein. Several approaches may be taken in utilizing the various embodiments of cannula 700, including its insertion into the opening 22 after container 200 has been placed into the patient working cavity 30 and before or after tissue specimen 20 is captured therein.

The cannula embodiment 770 of FIGS. 17-19 is also well-suited to accomplishing the various methods of the present disclosure as will be discussed below. Cannula 770 includes a main portion 772 having a proximal end 782 and a protector portion 774 having a distal end 784. As with the cannula protector portion 744 of FIGS. 16A-B, cannula protector portion 774 protects the bag or enclosure 200 from damage during tissue processing. Extension 776 is shown in the embodiment of FIGS. 17-19 as being an integral part of cannula protector portion 774 at cannula distal end 784. Extension 776 may also be a separate member that is attached to cannula distal end 784 and need not be made of the same material as the rest of cannula 770.

In the embodiment of FIGS. 17-19, extension 776 serves primarily to drive how tissue specimen 20 is cut or processed by cutter 400 during the methods disclosed herein. In particular, extension 776 will help to preferentially guide or position tissue specimen 20 such that cutter blade 408 is preferentially placed on a tangent to tissue specimen 20 near its outer surface 20a as seen in the schematic of FIG. 18B. As the cutter operates on specimen 20 in this manner, blade 408 creates a “peel” cut into specimen 20, much like the operation of an apple peeling machine, rather than “coring” into the specimen.

FIGS. 18B-C show tissue specimen 20 interacting with extension 776 in an end-on view (FIG. 18B) and a side view (FIG. 18C) of the FIGS. 17-19 embodiment. A radius of curvature r for extension 776 may be designed to be sufficiently smaller than an idealized radius R of tissue specimen 20 such that an upper extent of cutter 400 is a positive distance d relative to a tangent to specimen outer surface 20a as shown in FIG. 18C. In contrast, if extension 776 radius of curvature r is not sufficiently small relative to radius R of specimen 20, an upper extent of cutter 400 is a negative distance relative to a tangent to specimen outer surface 20a as shown by the distance −d in FIG. 18D is a positive value with reference to a tangent to specimen surface 20a, systems 100 employing the embodiments of FIGS. 17-19 will tend to ensure such a “peel” cut of specimen 20 is effected by cutter 400 rather than a “coring” type of cut.

Such a configuration helps to control the tissue cutting process and gives the physician or other user confidence that, in what can be a “blind” process if no visualization instruments are used, tissue specimen 20 is being processed in a reliable and effective manner. It also serves to protect container 200 from being damaged by blade 408 as extension 776 can generally be made more durable and rigid than protectors 776. In the cross-sectional view of FIG. 18A, enclosure 200 is shown surrounding cannula 770 to illustrate, in a fully tensioned configuration where tissue specimen is no longer in bag interior 204, how protector portion 774 protects container 200 from damage during cutting/morcellation of tissue specimen 20 as described above.

A protector portion 774 having or more protector elements 778 may also be included in the embodiment of cannula 770. Protector portion 774 can function as a guard to protect container 200 from damage during tissue specimen cutting and can also aid in guiding specimen 20 towards cutter blade 408. Protector elements 778 in this embodiment are integrated as part of or attached to cannula 770. A cannula proximal end 782 may incorporate a flared feature and/or handle, as discussed in connection with the embodiment of FIGS. 16A-B for ease of handling by a physician or other operator.

There can be anywhere from one to thirty or more protector elements 778 in this embodiment 770 of cannula protector portion 774 (four such elements 778 are shown in FIGS. 17-19). Elements 778 may be attached to the main portion 772 of cannula 770 or may be of an integral, seamless construction such that cannula 770 is a unitary member. Protector elements 778 are designed to provide enough stiffness to serve their guarding and guiding functions but have enough radial flexibility to bend and take on a variety of configurations. In one construction, protector elements 778 can be spring-loaded. Elements 778 may be made from a shape memory material, a spring steel alloy or other flexible metal such that the they can collectively have a small diameter when passing through the patient opening 22 or other port, and open to a larger diameter, either by activation or release from a constraint (or both) once inside the patient's working cavity 30. Elements 778 may include rubber or other relatively soft components on their distal tips to help prevent container damage, particularly from puncture, during the various steps in the methods described herein. In addition, a physician or other operator may optionally use grasper 500, via gripping or holding at least a portion of specimen 20, to pull the specimen axially towards the cutter 400. If the size of specimen 20 is large relative to that of protector portion 774, this action will cause protector elements 778 to bend out radially to accommodate the specimen 20 therewithin. This action combined with the design features that permit elements 778 to bias inward but spring outward radially as described above demonstrates the utility of this cannula embodiment 770.

Incorporated with or attached to protector elements 778 on the cannula protector portion 774 may be an additional protective layer or element 780. As seen in FIG. 17-19, layer 780 may take the form of relatively thin material, such as a durable plastic material including urethane, polyurethane, thermoplastic resins such as polyester (e.g., PET), silicone, PTFE, expanded PTFE (ePTFE), etc., that can be made to encapsulate or otherwise cover one or all surfaces of protector elements 778. Element 780 may be constructed of multiple layers or components. Element 780 is compliant enough and is designed to be able to sustain flexure motion as protector elements 778 bend during use, while still being durable so that container 200 is protected from damage by any of protector elements 778 during use. Element 780 may also partially or fully cover and/or encapsulate extension 776. The choice of material used and/or any surface or other treatment for element 780 may also facilitate ease of use as tissue specimen 20 comes in direct contact with element 780 during the methods of the present disclosure. For example, if element 780 is constructed from PFTE or ePFTE, it may be in either in an extruded tubular or (multiple) sheet form and can be engineered to have optimal isotropic or selectively anisotropic mechanical properties (such as, e.g., permeability, tensile strength, stiffness, tear resistance, abrasion resistance) to facilitate motion of tissue specimen 20 thereover, be biocompatible, and adequately to protect container 200 and protector elements 778. As described previously with respect to container 200, element 780 may be coated with and/or constructed using one or more layers of hydrophilic or hydrophobic materials and/or other lubricating materials or otherwise treated to provide a low-friction environment for interacting with tissue specimen 20. Such coatings or layers may be discrete and applied during manufacturing in sequential fashion (e.g., three-dimensional printing, other known deposition techniques) or may be in a composite or alloy-like form during manufacturing and/or as-fabricated. Having a low-friction and/or lubricious surface for element 780 can facilitate methods of tissue cutting and removal according to embodiments described herein, as the specimen 20 can spin or otherwise move relatively easily against element 780. Alternatively, all or a portion of element 780 may be constructed or treated to have a surface finish that is relatively rough, for example, near cutter blade 408 so that the tissue specimen is stable as it is being morcellated or cut. Differential surface finishes, treatments, or materials used for element 780 in any given embodiment, therefore, are contemplated herein.

In some embodiments, including those in components shown in FIGS. 16-19, features or elements can be included to limit the maximum opening size of the frustum or cone-shaped protector portions 744, 774 in their expanded configuration (as such configuration is exemplified in the views of FIGS. 16B, 17A-C, and 18). This may be desirable in methods disclosed herein where the body cavity 30 in which the systems are disposed may have sensitive organs or structures, for example, that could be damaged or otherwise negatively affected by a freely-expansive protector portion 744, 774. These limiting features, not shown in the Figures, may be, e.g., one or more circumferentially-disposed stiffening element(s) included around protector portions 744 and/or 774 in one or more locations, such as a distalmost edge, one or more intermediate locations, or even on a more proximal location thereon. Such limiting features can also serve to increase the hoop strength and resistance to radial deformation when the portions 744 and/or 774 take on their expanded shapes. In addition, such limiting features may also help to facilitate contraction of portions 744 and/or 774 during withdrawal of the components shown from the patient's body 31. Such stiffening elements may be incorporated as integral components of portions 744 and/or 774 and may, for example, comprise the same materials and design features of protector elements 746, 778 or may even be part of such protector elements.

Additional features, not shown, may also be incorporated into some embodiments, including those components shown in FIGS. 16, 17 and 18 of the present disclosure, to compress portions 744 and/or 774, and their components, for entry into a cavity such as a body cavity 30 and work in concert with protector elements 746 and/or 778. These features, similar to elements found in an umbrella, can include for example metallic or plastic snaps, hook and loop-type fasteners, zippers, or other mechanisms that may be released or activated by a physician or other user, manually or automatically, during deployment of portions 744 and 774.

FIG. 19 shows an embodiment of a system 100 of the present disclosure in which a cannula 770 as described above in connection with FIGS. 17-18 may be operated manually to effect cutting or morcellation of a tissue specimen by cutter 400. The embodiment of FIG. 19 illustrates one of several ways embodiments of the present inventions, and systems 100, may be configured. While the FIG. 19 embodiment shows manual operation of a version as described in connection with FIGS. 17-18, any of the embodiments, configurations, or component varieties contemplated by the present disclosure may be used with a manually operated cutting operation.

System 100 as shown in FIG. 19 includes a distal portion 110, proximal portion 120, cannula 770 integrated with guard elements, a tissue grasper or tenaculum 500, and a rotating cutter or morcellator 400. A container or bag 200 for tissue specimen 20 is not shown for purposes of illustrative clarity. On a proximal portion of 120 are disposed various control elements for operating the components described herein. A grabbing trigger 506 is operable by a physician or other user to open and close the teeth or jaws 512 of tissue grasper 500 and is disposed with grasper handle 504. A grasper axial trigger 506, which may include a ratcheting feature for ease and precision of use, may also be incorporated with grasper handle 504. In use, once grasper trigger 506 is operated to grab or hold at least a portion of tissue specimen 20 within teeth or jaws 510, the physician or other operator may activate axial trigger 506 to effect axial motion of grasper shaft 550 in a proximal direction to pull tissue specimen 20 towards cutter blade 408. This motion may be facilitated by a ratcheting mechanism (including spring 552 and other components not shown for clarity) to allow the physician or other user to move tissue specimen 20 in specific increments, as small as 1.0 mm or less (or greater if desired and as may be optionally selected by the physician or other user in some embodiments), during the procedure, to ensure reliable and safe operation.

It should be noted that although in FIG. 19 the embodiment described shows a handle 504 as being a part of tissue grasper 500, a handle of system 100 may be integrated as part of cannula 700, cutter 400, and still serve as a holding location for the physician or user to operate the various embodiments of system 100 as described herein. A handle may also be a separate component that is part of a system 100 but not incorporated as part of any given system component. As such, the labeling of handle 504 as part of grasper 500 is, while an embodiment of the systems described herein, is not meant to limit the location or configuration of a handle used in a more general sense to operate the systems described herein.

Disposed on the proximal portion 110 of the FIG. 19 system 100 embodiment is a component for manual operation of cutter 400. Here, a rotatable shaft 456 is disposed within a housing of system 100 and connected by a bevel gear system 458 (seen in schematic cross section in FIG. 19) to cutter 400. This shaft 456 is part of a hand crank 450 that also includes a handle 452 attached to wheel 454. Handle 452 and wheel 454 may be ergonomically optimized to facilitate ease of use; for instance, wheel 454 may have a platform-like surface on which the physician or other operator may rest his or her hand during a cranking maneuver to effect precision, minimize unwanted motion of system 100, and prevent operator fatigue. During the appropriate moment in the procedure as described variously herein, a physician or other operator may begin the tissue specimen cutting/processing step by rotating the cutter blade 408 by turning hand crank via the handle 452 and the relative motion of shaft 456 through bevel gear system 458. Of course, other versions of manually-powered embodiments, such as a moving foot pedal treadle system, ratcheting mechanisms, and the like, may be used in embodiments of the present disclosure instead of the hand crank 450 configuration as shown. The use of such a manual system as an alternative to an automated system or semi-automated system (both of which are also within the scope of the present disclosure) may afford greater control and precision when processing tissue specimens 20. Such a system may be of particular utility to cut through or otherwise process tissue specimens that are not homogeneous and that may have more dense or tough portions that require extra attention. Another advantage of the manual cutting system of FIG. 19 is that the physician or other operator may very readily reverse the direction of cutting, operate the cutter 400 in a “stop-start” fashion, etc. to maximize the effectiveness of the procedure. As discussed below, different embodiments of blade 408 that may be used with the present disclosure may be particularly amenable to use with a manually-operated cutter. It is within the scope of the present disclosure for other configurations of a manually operated cutter 400 to be utilized, and the particular configuration and components of the system as illustrated in the FIG. 19 embodiment should in no way be construed as limiting. For example, manually powered capability may be a feature of any of the components of system 100 disclosed herein, including combinations thereof (e.g., containers, manipulators, blades, guards, and their combinations etc. as may otherwise be described under the terms of power morcellation).

FIG. 20 schematically depicts an embodiment of system 100 illustrating a way that, in the context of a transvaginal procedure as described herein, advantageous tensioning may be effected without the necessity of rolling up or otherwise gathering the open end 206 of container 200 as tissue 20 is processed. In this example, the length of cannula 700, cutter/morcellator 400 and grasper or tenaculum 500 can be specified such that if container 200, with the specimen 20 therein, is pulled far enough towards the cannula distal end 752 that it would accommodate the distance required within the abdomen to pull the uterus or specimen 20 closer to the edge of the patient cavity 30 near the vaginal cuff and the desired zone of morcellation. This technique can be used instead of rolling up the container 200 as described elsewhere herein. Tension can be placed on the container 200 either by hand or with an apparatus (not shown) that controls the container tension. Container 200 may include one or more handles 292 (not shown) as described elsewhere herein, and such handles may be either permanently attached to bag 200 or may be affixed thereto during the procedure as the enclosure 200 is extracted from the patient's body 31. The embodiment shown in FIG. 20 may be configured for the same type of operation (no need to roll up container) in other, non-gynecological applications as discussed herein.

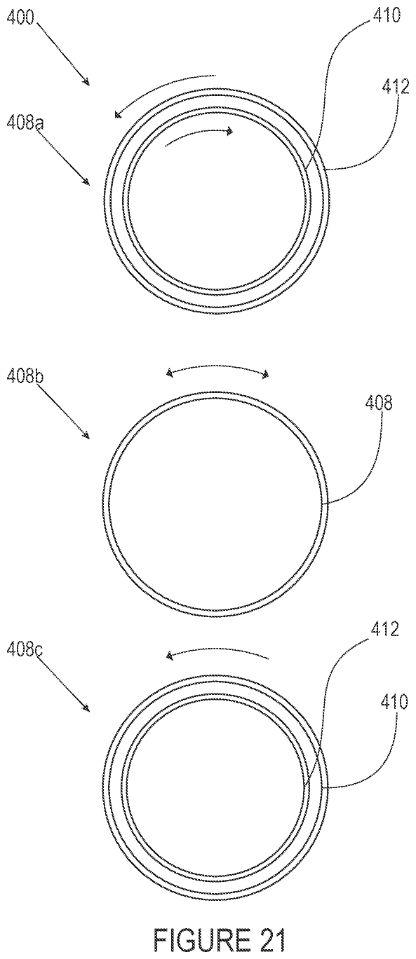

FIG. 21 depicts various views of cutter 400 embodiments that may be used in embodiments and methods of the present disclosure. Such cutter/morcellator 400 embodiments are described herein by way of example; other cutter embodiments, blade configurations, and designs may be used with systems and in methods of the present disclosure.

In the end-on view of cutter blade 408, cylindrical blade varieties 408a, 408b and 408c are shown in FIG. 21 in an end-on view. Blade 408a is an example of an opposing blade configuration in which an inner blade 410 component and an outer blade component 412 may be concentrically disposed and configured to rotate in opposite directions. The surface of such blade components 410, 412 may have different finishes, teeth types, or may be made of different materials to effect optimal cutting through what might be fibrous or difficult tissue within specimen 20, and may be particularly useful with a hand-operated cutting motion as described in connection with FIG. 19. Blade 408b is illustrative of a blade that can be operated in an oscillating or reversing rotational mode in addition to cutting in one direction only (all blade embodiments may be operated in unidirectional or oscillating modes). Blade 408b may be amenable to an automated and/or motor-driven cutter 400 to effect higher-rpm or oscillation frequencies for cutting through particular tissue types 20 of interest. Blade 408c is illustrative of a dual blade system in which one of blades 410, 412 is stationary during the cutter operation and one rotates as indicated. Such a blade configuration may assist the physician or user in having the cutter assist in obtaining greater purchase of tissue during the cutting process for a more stable and reliable operation, as the stationary blade can penetrate the tissue upon the application of axial force into the tissue as the other blade rotates to process the tissue. In all blade 408 embodiments of the present disclosure, the edge may be serrated, smooth, or have other features to optimize the cutting of tissue as described herein. The blade 408 may be equipped with additional capability, such as radio frequency (RF) energy application, to assist in the cutting operation (or alternatively other components of the systems herein may include a dedicated and separate RF tool). If RF energy is used on blade 408, it may be unnecessary to use rotational motion for blade 408 to cut through tissue. And although blades 408a, 408b and 408c are shown at the end of a cutter 400 in the form of a standard right cylinder so that its cross-sectional shape is that of a circle, cutter 400 and/or blade 408 may take on other shapes as appropriate, including those that have cross-sections representing a crescent, semicircle, irregular, etc., with or without RF-energy supplementation.

Another embodiment of a cutter blade 408 (not shown) involves a cylindrical structure similar to that of 408b but contains within its inner volume a rotating and/or oscillating blade, perhaps triangular in shape, that moves within the structure's inner volume to axially exit its distal end and cut tissue as it rotates. Such a blade may be an integral part of or attached to a second cylinder concentrically disposed within the first cylindrical structure's inner volume; the second cylinder serves as a carrier for the liner/triangular blade or blades of which the second cylinder is a part.

Other cutter 400 embodiments are within the scope of the present disclosure, including those that utilize optical (e.g., laser), vibrational, fluid (e.g., hydro-jet), or other modalities, singly or in combination with one another or with any of the means described above, to cut tissue as known to those of skill in the art.

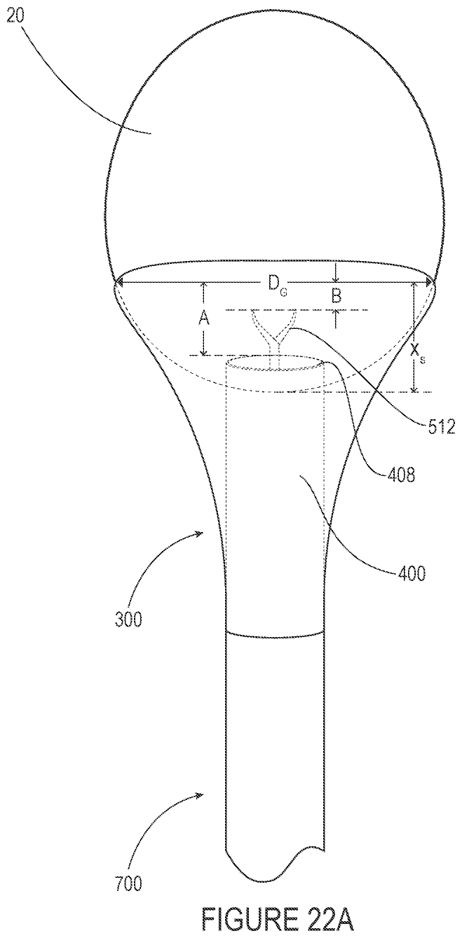

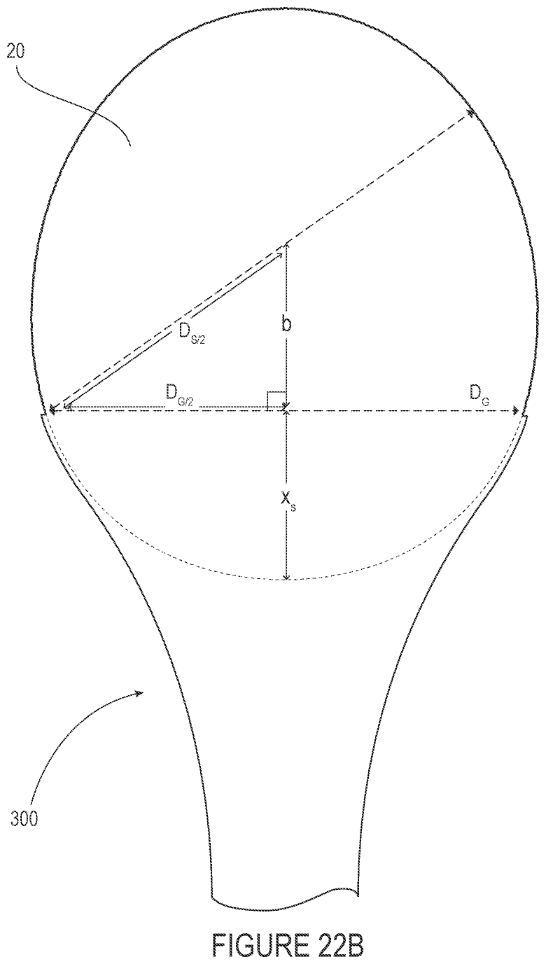

FIGS. 22A-B illustrate nondimensionalized spatial relationships useful in configuring systems and methods of the present disclosure so to help ensure an optimal clinical outcome. In these schematics, a cannula 700 distal end 752 is seen having a guard 300 integrated with or attached thereto such that the guard flares out to a maximum diameter. This is shown in FIG. 22A as plane D_{G}. Tissue specimen 20 is shown in apposition to cannula distal end 752 and partially within the volume created by cannula distal end 752 to a depth xₛ. Specimen 20 has a maximum diameter D_{S}. A distal portion 510 of tissue grasper or tenaculum 500, showing jaws 512, is seen extending out of cutter central lumen 402 in the vicinity of blade 408 and generally in alignment with the central axes of cannula 700 and cutter 400. The symbol A in FIG. 22 represents a distance between a line representing a plane of the guard at its maximum diameter D_{G} and the distal extent of cutter 400 at blade 408. The symbol B in FIG. 22 represents a distance between a line representing a plane of the guard at its maximum diameter D_{G} and a distal end 548 of grasper 500 at jaws 512. D_{G} can be specified so that it matches the size of specimen 20 and so that it controls xₛ, the depth of the specimen into the guard as measured from the line at D_{G}. In one embodiment, both distances A and B are less than xₛ. Such an embodiment helps to ensure the specimen 20, which is relatively large compared to the guard 300, comes into contact with both the cutter 400 (particularly blade 408) and tissue grasper 500.

FIG. 22B in turn exhibits how such parameters may be idealized to solve for a specimen depth xₛ. A right triangle is shown having sides D_{G}/2 and b, and a hypotenuse D_{S}/2.

The Pythagorean theorem posits the sum of the squares of the two sides of a right triangle equals the square of the triangle's hypotenuse. In this context:

$${( \frac{{D}_{G}}{2} )}^{2} + {b}^{2} = {( \frac{{D}_{s}}{2} )}^{2}$$

In FIG. 22, specimen depth xₛ is represented by

$${x}_{s} = \frac{{D}_{s}}{2} - b$$

such that

$$b = \sqrt{{( \frac{{D}_{s}}{2} )}^{2} - {( \frac{{D}_{G}}{2} )}^{2}}$$

And thus one can solve for specimen depth xₛ by:

$${x}_{s} = \frac{{D}_{s}}{2} - \sqrt{{( \frac{{D}_{s}}{2} )}^{2} - {( \frac{{D}_{G}}{2} )}^{2}}$$

Therefore, one may calculate specimen depth xₛ, knowing that an ideal solution involves a specimen depth being less than dimensions A and B in FIG. 22A, by designing the maximum diameter D_{G} of guard 300 relative to the maximum diameter D_{S} of tissue specimen 20 expected to be used therewith.

As shown in the idealized way in FIGS. 22A-B, the symmetric cone afforded by guard 300 and how specimen 20 lines up therewith would result in a “coring” type of specimen cutting as opposed to the “peeling” type of specimen cutting as described herein in connection with the embodiments of FIGS. 17-19. Therefore, these spatial relationships can be calculated in the context of a non-symmetrical conical guard protector portion where a tangent to the tissue specimen 20 surface 20a is placed at the blade 408. In any configuration, however, the desire for the dimensions of a cone-shaped guard component to allow for a relatively large tissue specimen to come into contact with the cutter 400 and grasper 500—components that are not designed to protrude beyond the opening of the guard—dictates ideal dimensions of the guard cone. It is also contemplated that because either or both of cutter 400 and grasper 500 may also be usefully disposed, even if only for a brief moment during use, above the line representing a plane of the guard at its maximum diameter D_{G}, the distances A and B may have negative values relative to D_{G}.

Exemplary Methods of Use

FIGS. 23A-F illustrate a method of use of the systems 100 of the present disclosure. It is presented as a method of performing a transvaginal procedure, such as a hysterectomy, but the techniques detailed herein may be used elsewhere in the body without departing from the spirit of the embodiments disclosed herein.

Prior to the steps outlined below in connection with FIGS. 23A-F, a physician or surgeon will prepare a patient 31 by using known techniques to prepare the tissue specimen 20, which in this example may contain a uterus and possibly other tissue and organs (such as one or both ovaries, fallopian tubes, connective tissue, etc.), for removal from the body. As known by those of skill in the art, this may be performed via any number of approaches, such as, e.g., by performing a minimally invasive hysterectomy, perhaps robotically, through the use of standard laparoscopic instruments. This is schematically represented in FIG. 23A: one of any number of laparoscopic instruments 610, which may involve a tissue manipulator, cutter, or similar tool, has gained access via a surgically-created port to a patient's pelvic cavity 30. Cavity 30 may be insufflated using known techniques to create working and visualization space in cavity 30 via, e.g., insufflation instruments delivered through one or more ports in the patient's body 31. A laparoscope 612 is shown accessing cavity 30 by a second surgically-created port to allow the surgeon to visualize the procedure and his or her use of the tools. A uterine manipulator 640 (such as the VCARE DX uterine manipulator sold by ConMed Corporation of Utica, N.Y.) is shown disposed in the patient's vagina 32. At the moment in time represented by the schematic of FIG. 23A, tissue specimen in the form of a uterus 20 has been excised from its connective tissue, including the level of the cervix, and is being held in the pelvic cavity 30 by instrument 610. A distal end of the uterine manipulator 640 may have been utilized to assist in the process of preparing the uterus 20 for removal as known by those of skill in the art. For ease of illustration, other tissue such as one or both ovaries, the fallopian tubes, and other tissue intended for removal are not shown but may be considered part of the tissue specimen 20 referred to and shown herein as uterus 20.

FIG. 23B shows a transvaginal insertion of bag or enclosure 200 into the vaginal opening, through the vagina 32 and into pelvic cavity 30. Uterine manipulator 640 has been retracted and removed from the body through the vagina. A tether, drawstring, or other element 216 is shown extending through the vagina 32 and out of the vaginal opening 34 to provide for manipulation of container 200 by the physician or other user. Tether or element 216 may be flexible or may have a stiffer construction, in particular in its column stiffness, to facilitate optimal container manipulation. Uterus 20 is shown being captured by container 200 and placed into an interior 204 by the assistance of tool 610.

Occluder 650, and as shown, vaginal occluder 650, can then be placed in vagina 32 as shown in FIG. 23B, with tether 216 optionally extending therethrough, to establish/reestablish and maintain insufflation of cavity 30 as known to those of skill in the art through, e.g., the introduction of a gas such as carbon dioxide through one of the surgically-created ports in the pelvic or abdominal cavity 30. In other embodiments, a high flow insufflator may be used to maintain insufflation of cavity 30 even as gas escapes through the vaginal canal, without use of an occluder or other means, due to the higher gas flow rates it affords.

Generally, occluder 650 may be placed trans-vaginally, trans-pelvically, or via any other body orifice or surgical incision, and may be in a variety of lengths (for example, between about 30.0 mm or less and about 300 mm or more, including up to approximately 1,000 mm or more) and diameters (for example, between about 10.0 mm and about 80.0 mm) to accommodate a variety of surgical or natural openings or ports. In some embodiments, occluder 650 provides a snug fit against the walls of the opening or port into which it is disposed, optionally through the use of a seal, and by choosing the appropriate size. When a snug fit between occluder 650 and the opening or port is accomplished, fluid leakage around an outer diameter of sheath may be minimized or even eliminated. This may be useful when it is desirable to achieve and/or maintain pneumo insufflation of a body cavity 30 containing specimen 20 to be captured and removed. Occluder 650 may contain a central lumen through which other instruments may be disposed or deployed, such as tether, drawstring or similar instrument 216, a tissue cutter 400, a tissue grasper or tenaculum 500, or the like. Occluder 650 may include a blade guard to protect healthy tissues as well as the container 200 from accidental damage.

In FIG. 23C, the physician or other operator uses tools such as tether 216 and possibly tool 610 to manipulate container 200 with specimen 20 placed therein so that container opening 206 defined by container edge 206A is pulled through the vagina 32 and out the vaginal opening 34. Container may next be tensioned using various techniques as described herein to pull the uterus 20 near or even against the vaginal cuff within pelvic cavity 30. FIG. 23C depicts container 200 now oriented such that the opening 206 is outside the patient's body 31. Container edge 206a may be rolled in on itself in the direction shown to foreshorten container 200, effecting the aforementioned tension to bring uterus 20 in abutment with, adjacent to, or at least closer to the patient's vaginal cuff in the pelvic cavity 30. Instruments 610 and 612 may remain in their ports as shown to assist the physician or other user in completing the procedure.

Depending on the type of insufflation that may be used during this exemplary procedure, container 200 as tensioned and put into apposition against the vaginal wall at opening 34 may rapidly or even instantaneously produce an adequate seal against fluid, including insufflation gas, from leaking out through the vaginal canal and opening 34. As such, any plug used to reestablish insufflation as described above, including occluder 650, may then be removed from the patient's body 31. In some embodiments occluder 650 may be expelled from opening 34 simply by the action of the physician or other operator pulling container edge 206a and opening 206 out of the patient's body 31 through vagina 32 and opening 34. This placement of container 200 as shown in FIG. 23C allows for insufflation (or re-insufflation) of the pelvic cavity 30 to provide an optimal viewing and working environment for the physician or other user to complete the exemplary procedure, perform necessary suturing, tissue cauterization, etc., without the need of additional tools.

Turning to FIGS. 23D-F, a cutter/morcellator 400 is shown accessing the interior 204 of enclosure 200 through cannula 700 while proper tensioning is maintained on container 200 to keep uterus 20 in position for removal. Any guard 300 or protector structure elements, such as those described herein, whether integrally forming a cannula protector portion 744 or attached to cannula at its distal end 752. Cannula 700 has been inserted into the patient's vagina through opening 34, via interior 204 of the previously-placed container 200, either first or simultaneously with cutter 400. Any guard 300 or protector feature present may expand against the vaginal wall near the vaginal cuff in the vicinity of cavity 30, or wholly within cavity 30, to facilitate insufflation if needed. FIG. 23E shown distal portions 752, 548 of cannula 700 and cutter 400, respectively, approaching uterus 20. Instruments 610, 612 are not shown for clarity of illustration but may be utilized at any time to aid in positioning uterus 20 as needed. Extension 776 of the cannula embodiment 770 is shown in FIG. 23E as disposed within bag interior 206 and adjacent uterus 20 so to preferentially facilitate bringing the uterus into the gap 788 created between cutter distal end 548 and extension 776 as described in connection with the embodiment 770 of cannula described herein with reference to FIGS. 17-19. It should be noted that other cannula embodiments described herein, including without limitation cannula 740, may be used in the procedure described in connection with FIGS. 23A-F as well as other procedures and methods of the present disclosure.

FIG. 23E also shows a tissue grasper or tenaculum 500 being disposed in the central lumen 402 of cutter 400 so that Such a grasper 500 may be a custom, heavy duty tool, or one of many commercially available to physicians. Grasper gripping elements or jaws 510 grasps a portion of uterus 20 and the operator may retract grasper axially and/or maintain axial tension on grasper 500 to bring uterus 20 closer to cutter blade 408, and the cutter may be then activated after or during this tension being applied through grasper 500 to cut uterus 20. As cutter 400 is operated, either manually or automatically as described herein, the physician or user may continue to apply tension to container 200 and axial tension on grasper 500 to keep uterus 20 in contact with blade 408. Uterine tissue 20 will be cut/morcellated to the extent that the rotating cutting action in connection with the applied tension, pulls the tissue through lumen 402 of cutter and out of the patient's body 31 at a proximal portion 422. This may be accomplished relatively quickly. During this tissue cutting and removal process, guard 300 or features such as protector elements 746, 778, enclosing element 780 and extension 776 described elsewhere herein serve to protect container 200 from damage, aiding the function of enclosure 200 in the present disclosure to fully contain the tissue specimen 20, including any cancerous or pre-cancerous cells associated therewith, and keep such tissue from being left in the pelvic cavity 30, vagina 32, surgical ports, etc. in undesirable fashion.

After uterus 20 is processed as described above and when tissue specimen 20 is or has become by virtue of the cutting action of cutter 400 small enough to be removed from the patient's body 31, cutter 400 and cannula 700 may be withdrawn from the interior 206 of container 200. Simultaneously with or after the withdrawal of cutter 400 and cannula 700, container 200 may be pulled in its entirety out of the patient's body 31 through vagina 32 and vaginal opening 34. The procedure may then be completed with routine suturing, cauterization, etc. as necessary.

One advantage of the transvaginal approaches described herein, including the method described in connection with FIGS. 23A-F, is that by processing tissue within container 200 but through the vagina 32, less cutting is necessary. Morcellating or processing tissue using other techniques, such as by a surgical incision (i.e. a “mini-laparotomy”) or by use of a PNEUMOLINER sold by Olympus America, Inc., can require an incision on the order of about 5 cm or greater in size. In comparison, transvaginal removal of tissue does not require as much morcellation or tissue processing/cutting, as the vagina is a relatively large, flexible port, naturally occurring. Moreover, minimal cutting/processing of tissue specimens 20, such as the uterus 20, presents a faster process as the tissue need be morcellated only to the extent that it can be readily removed through the vagina. Larger, less processed portions of the tissue sample removed via these techniques may be more attractive to pathologists studying the tissue specimen 20 as the larger portions are likely to have suffered less violence as they are removed.

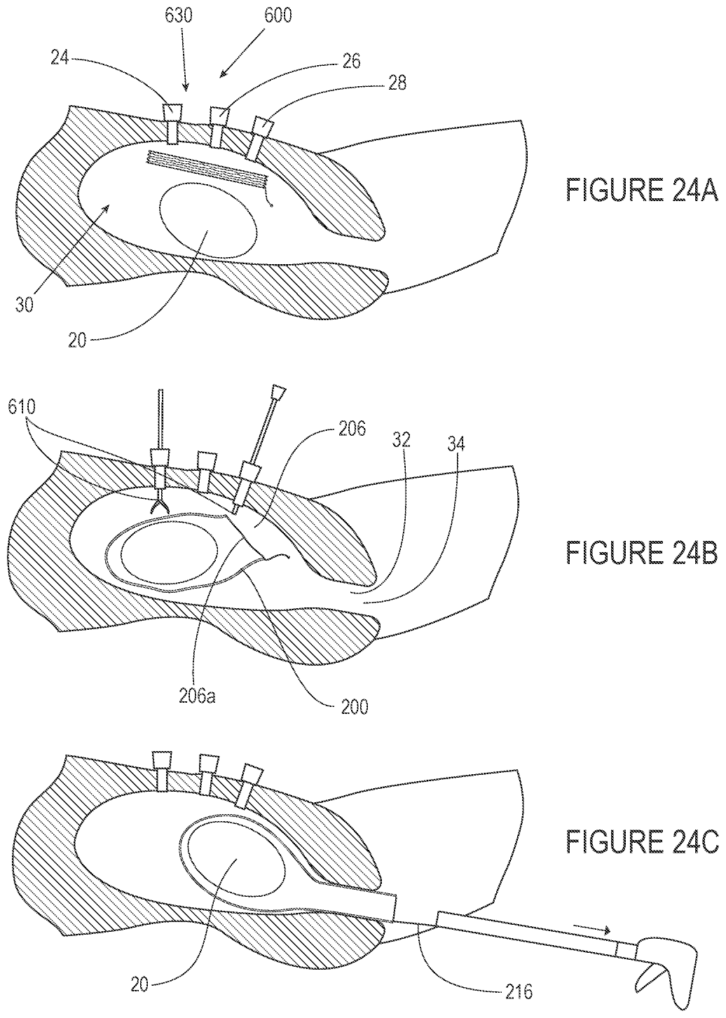

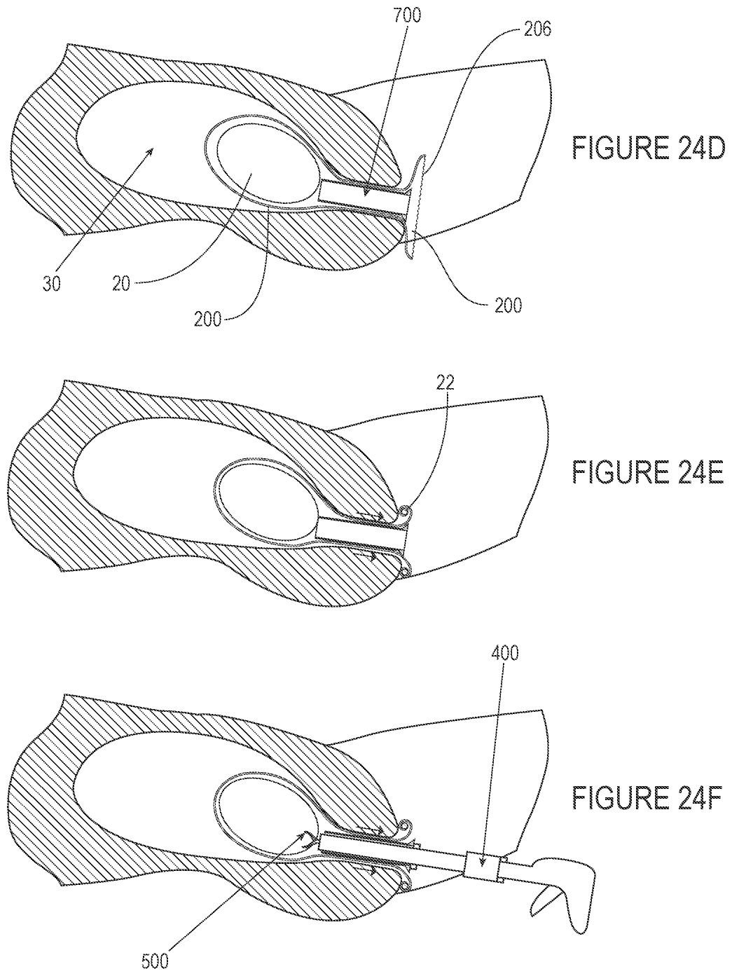

Another transvaginal method of use is shown in connection with FIGS. 24A-F. In this embodiment, laparoscopic techniques are utilized and the uterus 20 is detached using traditional methods. A collapsible containment device or bag 200 is then introduced into the patient's pelvic cavity 30 through the vagina 32, through one of ports 24, 26 or 28 as shown in FIG. 24A, or through a small abdominal incision such as a port site with the port momentarily removed. The physician or other user places specimen 20 into the container interior 206 through the help of a robot and/or with instruments such as instrument 610, 612 that are guided through the abdominal ports 22 or instruments guiding through the vagina 32. An end of container containing opening 206 is then pulled out by the physician or other operator through the vagina (or other port) with an instrument such as, e.g., a grasper 500 pulling on tether 216 connected to container 200 as shown in FIG. 24C. In this embodiment, a cannula 700 is next introduced into the vagina 32 as shown in FIG. 24D. Cannula 700 may be of the appropriate diameter and length for the patient 31 being treated, and serves to create a smooth working channel for the cutter/morcellation equipment 400. An optional guard 300 (not shown) or a cannula equipped with protective features described herein may also be used to help guide the tissue specimen 20 into the cutter 400 and to protect the enclosure 200 from being damaged. Such protective features may also be part of the cutter 400 and/or tissue grasper 500.

FIG. 24E shows that by pulling edge 206a of container with respect to the cannula in a proximal direction away from pelvic cavity 30, which may be done prior to insertion of cannula 700, during or after insertion of cannula 700, the physician or other operator can concomitantly draw the specimen 20 closer to or against the vaginal cuff, and when cannula 700 is in place, near or against cannula distal end 752 (and if in place, blade 408 of cutter 400) while also providing the desired tension for tissue cutting as described elsewhere herein. As described below, an optional ring 294 can be placed on container edge 206a to aid the physician or other user in this step or other tensioning techniques and equipment as described elsewhere herein may be employed. Cutter/morcellator 400 is placed through cannula lumen 702, either simultaneously with the introduction of cannula 700 or afterwards, so that the uterus or specimen 20 can be morcellated. Tissue grasper or tenaculum 500 is shown disposed through cutter lumen 402 for grasping uterus 20 to align it with cutter blade 408 and for applying additional tension to facilitate specimen processing.

An optional ring or rolling device 294 can be placed or inserted on or integral with the edge 206a of container 200 to aid the physician or other user in the step of pulling on container 200 to bring specimen 20 closer to cannula distal end 752. In FIG. 24E ring 294 is seen rolled towards the container interior 204 or towards an exterior of container 200. Rolling device is represented in FIG. 24 as a toroid having a circular cross section; however, other geometric shapes can be used to bunch, wrap or wind container 200 to create any desired tension.

In addition to the use of various protective features as described herein either as a guard 300 or protective features on cannula 700 and/or cutter, application by the physician or other user of tension on container 200 can prevent the container from being drawn towards the blade 408, avoiding damage to container 200. Other features such as, e.g., mechanical stops, detents, pins, notches, or other mechanisms can be used to position the edge of cutter/morcellator blade 408 precisely relative to the cannula distal end 752. Cutter blade 408 can be positioned within about 3.0 mm to about 5.0 mm of the cannula distal tip 752; alternatively, cutter blade 408 can be positioned within about 0.0 mm to about 50.0 mm of cannula distal tip 752. Additionally, after it is advanced through the cannula lumen 702, blade 408 may be positioned within about 3.0 mm to about 5.0 mm from the cannula distal tip 752 or within about 0.0 mm and about 50.0 mm from the cannula distal tip 752. There can also be a precise spatial—axial or radial—relationship between the grasper 500 and the cutter blade 408 as well as between the tenaculum 500, including jaws 512, and the cannula distal end 752. For example, a maximum distance between the far reach of the tenaculum grips 512 and the blade 408 could be about 10.0 mm, but could range from about 0.0 mm to about 250.0 mm. The maximum distance between the maximum reach of the tissue grasper/tenaculum grips 512 and the end of the cannula could be between about 0.00 mm and about 5.0 mm, but could range from about 0.0 mm to about 250.0 mm.

As cutting/morcellation progresses according to this method, portions of tissue specimen 20 will be transported through the cannula central lumen 702 and out of the patient's body 31; in this case, the vagina 32. During the cutting process, the uterus 20 is enclosed safely within bag interior 204 to achieve the objectives described herein. FIG. 24F illustrates that morcellation only needs to be performed until the uterus 20 is small enough to be pulled out of the port, in this example, vagina 32 and vaginal opening 34. Once cutting and tissue specimen 20 removal is complete, cutter 400, tenaculum 500, and container 200 may be removed from the patient's body 31 and the procedure may be completed as described herein.

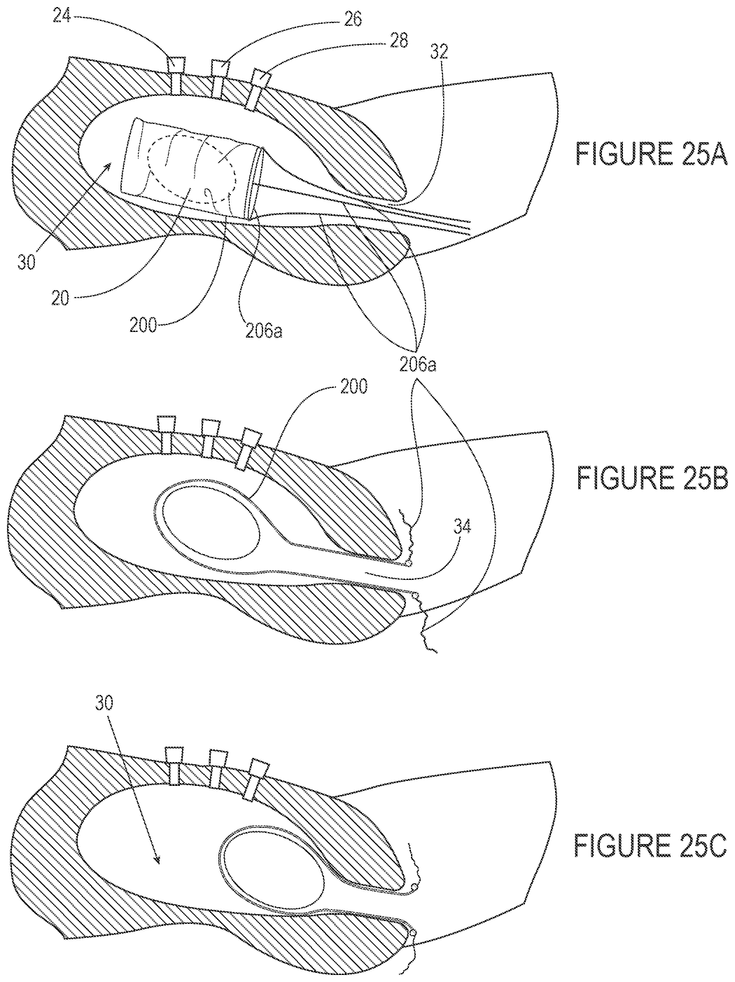

FIGS. 25A-C depict another method of using the various systems and components of the present disclosure in a hysterectomy context. As with the embodiments of FIGS. 23-24, standard techniques may be used to access and detach uterus 20 from within the pelvic cavity 30, and the techniques described herein may be used to deploy container 200, capture tissue specimen/uterus 20 within the container interior 204, optionally employ cannula 700 and/or tissue grasper 500 and employ cutter 400 to cut and process uterus 20 and remove it through the vagina 32 as previously described. In the embodiment of FIGS. 25A-C, however, bag 200 contains multiple tethers, strings or flexible wires 216 attached to or incorporated as part of container distal end 255 near opening 206. Three such tethers 216 are shown in FIGS. 25A-C as extending through the vagina 32 out through the vaginal opening 34 and assist the physician or other user in entrapping and capturing specimen 20, particularly if instruments 610 deployed from surgical ports are of little help. As with other systems and methods described herein, the physician or other user prepares the tissue specimen 20, in this case uterus 20, using standard techniques; typically laparoscopic and/or robotic using tools 610 and/or scopes 612. Container 200, which may be stored in compact form (e.g., in a collapsed, accordion-like configuration or rolled to a small diameter sufficient for deployment through a port) is deployed into the patient's pelvic cavity 30 either through the vagina 32 or via another port, such as one surgically created in the pelvic wall. Alternatively, small members made, e.g., of plastic or thin cloth (not shown) can be included so to aid in maintaining container 200 in a compact or collapsed position. These members or ties may be engineered to break or detach under sufficient force applied on the tethers or strings 216 so to fully deploy enclosure 200 for specimen capture.

Next, the physician or other user, via one or more of tethers 216 and/or tools 610, 612 and optionally via tools introduced transvaginally (not shown) or laparoscopically, unfurls or opens container 200 captures uterus 20 and places it into container interior 204 through opening 206. FIG. 25A depicts the uterus 20 captured wholly within container interior 204 and all three tethers 216 leading out of cavity 30, through vagina 32 and out of the patient's body 31 via vaginal opening 34. If, e.g., container 200 happens to be positioned on the opposite side of the specimen 20 from the vaginal port, the multiple strings or tethers—anywhere from about two to about 10 or more—may be utilized by the physician or other user to help capture specimen 20 within container interior 204. FIGS. 25A-B show how and then container edge 206a may be drawn towards the vaginal cuff, pulling container 200 and captured uterus 20 along by the physician or other user pulling or tensioning the ends of the strings/tethers 216; this in turn tensions container as described elsewhere. At this point, the specimen 20 may be cut/morcellated/processed and removed via the techniques described herein via use of cutter 400 and optionally grasper 500, and container 200 may be removed from the vagina 32 through opening 34 and routine items may be attended to in order to complete the procedure.

Rather than being flexible or string-like, one or more of tethers 216 in the embodiment shown in FIGS. 25A-C or any of the others described herein may be relatively stiff or wire-like. This embodiment can aid the physician or other operator in the step of capturing specimen 20 with less or even no assistance from additional ports, such as ports in the example of FIGS. 25A-C created in the patient's pelvic wall through which one or more tools 610 or scopes 612 may be deployed. In one mode of operation, for example, relatively stiff guides 216 enable the physician or other operator, working with guides 216 as deployed through a natural port such as vagina 32, to place the container 200 on a distalmost or far side of the uterus relative to the vaginal cuff, by placing the uterus 20 directly between the container 200 and the vaginal cuff at the former location of the cervix in the pelvic cavity 30. Once enclosure 200 is properly situated, these relatively stiff members 216 can be pulled in an axial direction out of the patient's body 31 by the physician or other user, and the uterus 20 can be captured, pulled towards and even against the vaginal cuff within container interior 204 and processed or morcellated as described elsewhere herein.

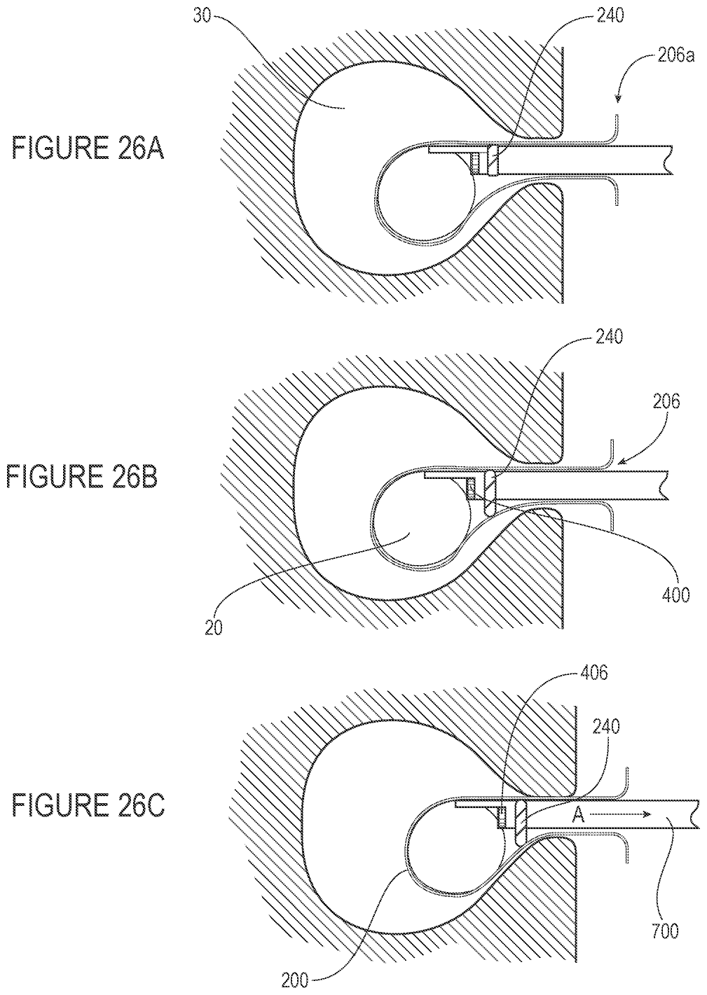

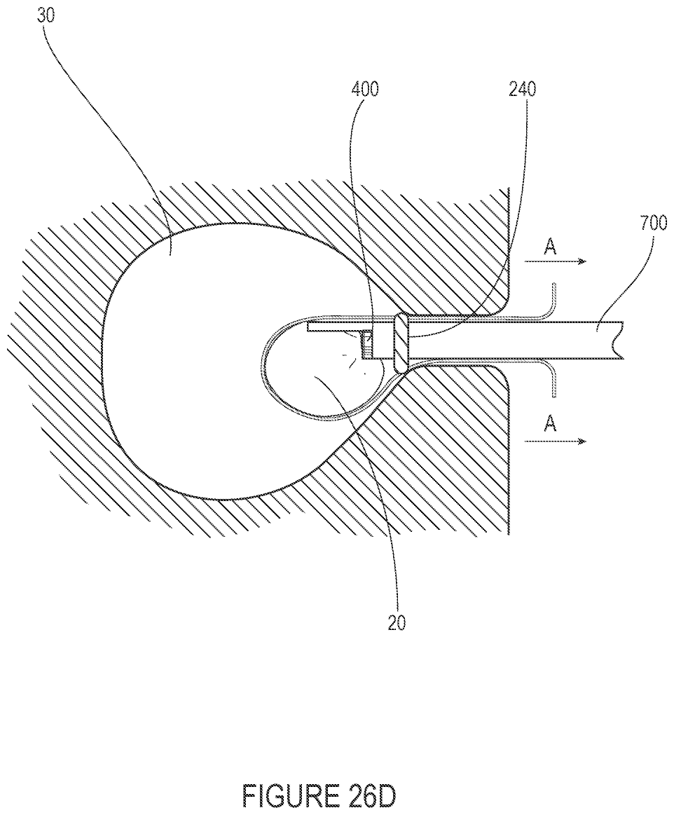

FIGS. 26-28 schematically depict additional embodiments and methods of their use. FIG. 26A depicts a system 100 of the present disclosure deployed in a patient's body cavity 30, such as a pelvic or abdominal cavity, with tissue specimen 20 captured within container 200, and container opening 206 is disposed outside the patient's body 31 and extending from the port (natural or surgically-created) so that container edge 206a placed as shown. Cannula 700 with cutter 400 are also shown as disposed within container interior 204, consistent with other embodiments and methods described herein.

An additional component in the form of a locking member or balloon 240 is seen as disposed on or around cannula 700 in the embodiments of FIGS. 26-28. While locking member or mechanism 240 is described herein as a balloon having an expandable surface when inflated, other locking components 240 that may be expanded, moved or actuated by fluid means or even by one or more alternative mechanisms, including, e.g., pneumatic pistons, motor-driven lead screws, expanding linkages similar to those used to open and close an umbrella, and the like are contemplated. The description herein is presented in the context of a balloon locking mechanism 240 although such other forms of a locking mechanism may be used.

More than one locking member or balloon 240 may be present in embodiments of the present disclosure, and for purposes of illustration only one balloon is described below. Balloon 240 may be inflated from an uninflated state shown in FIG. 26A to an inflated state shown in, e.g., FIGS. 26B-D to effect its purpose during use. While not shown in the figures, the one or more balloons 240 may be inflated by use of an inflation port, channel, tube or other means attached to an interior 242 of balloon 240. This port, channel, tube or other means may be in fluid communication with a source of inflation fluid (e.g., gas, saline, or other suitable fluid) that may be controlled by a physician or other user to inflate balloon 240 in a controlled manner as desired during methods of use. Balloon 240 may also in some embodiments be deflatable such that a precise amount of inflation (in case of, e.g., inadvertent over inflation or the need for repositioning system 100) may be employed to maximize the effectiveness of system 100 during use. A valve may or may not be present to provide additional control of balloon 240, and inflation/deflation may be controlled manually or automatically, under, e.g., pressure or volume control (via use of devices such as an ENDOFLATOR, etc.).

When locking member 240 is a balloon, it may be made from any suitable medical grade biocompatible material, including urethanes, silicones, plastics of various types, elastomers, PTFE and ePTFE, and be made of single- or multi-ply construction. Balloon 240 may include coatings and/or surface treatments, particular on its outer surface, so to aid it in performing its role as described below during methods of the present disclosure. Balloon 240 may be affixed to cannula 700 or other component by methods known by those of skill in the art, including, e.g., the use of adhesives, welding, mechanical fasteners, or combinations thereof, or balloon may be integrally formed with cannula 700 or other component. Balloon 240 may also include a material on its outer surface, such as, e.g., a covering of a rigid material, a surface treatment, panels, rigid sections, etc. Other forms of locking mechanism 240 may also include such elements.

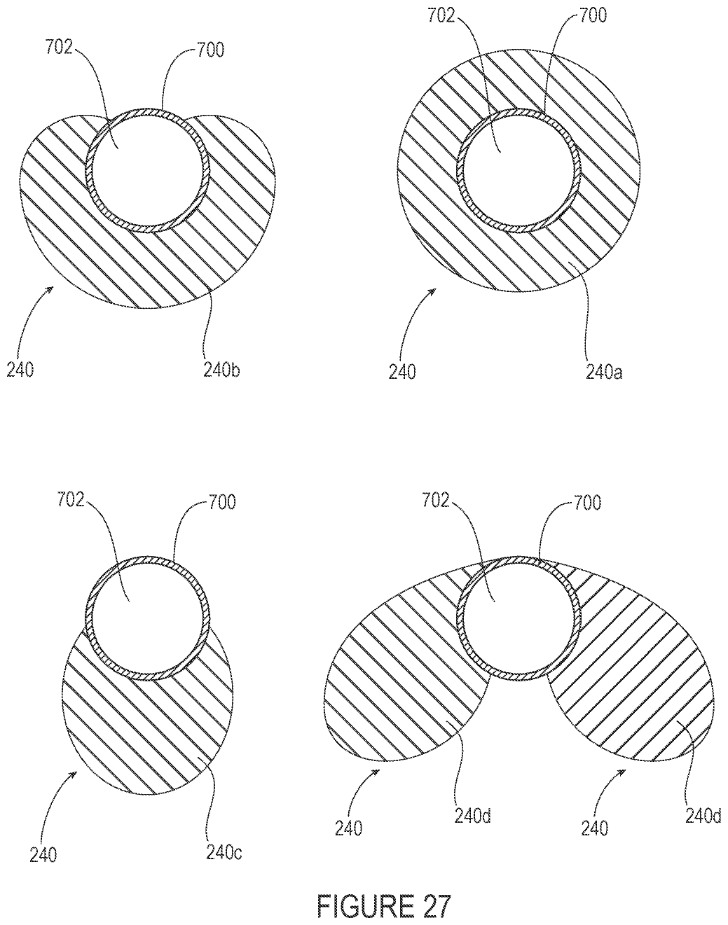

FIG. 26A depicts a locking member in the form of a single balloon 240 disposed on cannula distal end 752. The precise location of balloon 240 on cannula 700 and/or on other components usable in the present disclosure, and the actual number of balloons used may vary, as may the shape of the balloon(s) when inflated. For instance, between one and five or more balloons may be used, in identical or varying shapes, in system 100 depending on the anatomy into which the system is disposed and the methods and steps used in performing the tissue capture and removal procedure. As may be seen in the examples depicted in the end-on views of balloon 240 embodiments disposed over a cannula 700 in FIG. 27, balloon 240 may be of a simple toroidal shape 240a, a semi-circular shape 240b that does not extend around the entire circumference of cannula 700. A single lobe balloon 240c is also seen along with a dual lobe balloon 240d. Such lobed configurations for balloon 240 may be useful in applications in which a preferential apposition against body tissue or other components of system 100 is desired, such as when the anatomy of the patient requires. Three, four or more lobed balloon configurations are contemplated for certain embodiments. Other shapes amenable to optimized clinical use, such as tapered or crescent shapes and the like may also be used.

After system 100 having locking member or balloon 240 is placed in the desired location by a physician or other user and tissue specimen 20 is captured within container 200 as shown in FIG. 26A, balloon 240 may be inflated as depicted in FIG. 26B. The precise timing of balloon inflation may vary according to the procedure being undertaken and physician preference, but generally it will be inflated prior to the tissue cutting or morcellation process.

Recall that in systems of the present disclosure and accompanying methods, it is useful to employ components and techniques to bring tissue specimen 20 into close proximity of or apposition with cutter 400; namely, blade 408. As previously discussed, this may be accomplished by several means: hand or automatic tensioning of bag 200 so to pull specimen 20 closer to cutter 200, the use of a tenaculum or grasper 500 to pull specimen closer to cutter 200, etc. Balloon 240 affords yet another tool, which may be used singly or in combination with other techniques (e.g., bag tensioning) or components (e.g., grasper 500) to help the physician or other user to achieve this same objective. Ensuring that specimen 20 is placed kept into close proximity or contact with cutter 400 within container interior for tissue processing is a useful aspect of the present disclosure. In one respect, balloon or locking member 240 allows cutter 400 and cannula 700 to be locked in in the direction of the body port 22 so that when tension is applied to the container 200 (e.g., by a physician or other user pulling), the cutter 400 and cannula 700 remain locked in to provide counter-tension to the system. FIG. 26B shows that tissue specimen 20 has been moved into apposition with blade 408 by, e.g., tensioning bag 200 and/or by proximal movement of cannula 700. An advantage of balloon 240 is that users of systems 100 of the present disclosure that include one or more balloons may not need a grasper 500 in order to effect the proper apposition and tensioning of specimen 20 as desired. This provides an alternative, simple procedure capture, cut and remove tissue specimen 20. In fact, in some embodiments, balloon 240 may replace altogether the need for grasper 500. An embodiment that does or does not have the grasper can incorporate a uterine manipulator (not shown) that can be inserted through a central lumen of the cannula or the cutter.

As the physician or other user moves components of system 100 (e.g., cannula 700 and/or container 200) proximally in a direction A so to pull specimen 20 close to or in apposition with blade 408, balloon 240 as shown in FIG. 26C concomitantly moves in the same proximal direction until it abuts the patient's tissue surface within cavity 30. In this fashion, balloon 240 is “anchored” against the patient's body tissue in the cavity 30, for instance, against a cervical opening in a pelvic cavity. This frees the physician or other user from having to hold cutter 400 in a particular axial location or position during the cutting process. As a result, the physician or other user may concentrate more fully on tensioning or pulling bag 200 as described elsewhere in the present disclosure and to operate cutter to process specimen 20. FIG. 26D depicts this tensioning on bag (as shown by arrows A) outside the patient's body 31 as the cutter 400 disposed inside cannula lumen 702 is activated to cut or morcellate specimen 20. Balloon 240 serves to anchor the system 100 in place as seen in FIG. 26D, preventing cannula 700 from otherwise moving proximally in the direction of arrows A; this provides counter-traction such that the bag 200 is applying force on the tissue specimen 200 in the direction of cutter 400.

In addition to obviating the need for a tissue grasper 500, systems of the present disclosure employing one or more balloons affords several other advantages, including serving as a guard by the balloon or balloons acting as a barrier between cutter 400 (especially blade 408), optionally grasper 500, and the container interior 204 into which the cutter is disposed and the grasper may be disposed. The balloon or balloon may be properly sized and located on or near the distal end 752 of cannula 700 to create geometric tenting points that physically prevent container 200 from coming into contact with, or even close to, blade 408 or the optional grasper 500, including jaws 510. As such, these systems may also supplement the function of a guard 300 or protector feature as discussed herein, and may in some embodiments obviate the need for a guard or protector feature altogether, increasing simplicity of operation, lowering system cost, reducing procedure time, etc. The simplicity of such a system 100 employing one or more balloons is a particularly advantageous feature, as during a method of use, the physician or other operator engages the cutter 400 to process the tissue specimen 20 once the system is in place in the patient's body 31 and pulling on container (via any number of techniques discussed herein) to maintain the desired tension, complete the tissue morcellation process, and remove the container from the patient's body 31. Such a process may be used in any of the methods or applications described herein, including gynecological procedures such as hysterectomies, etc.

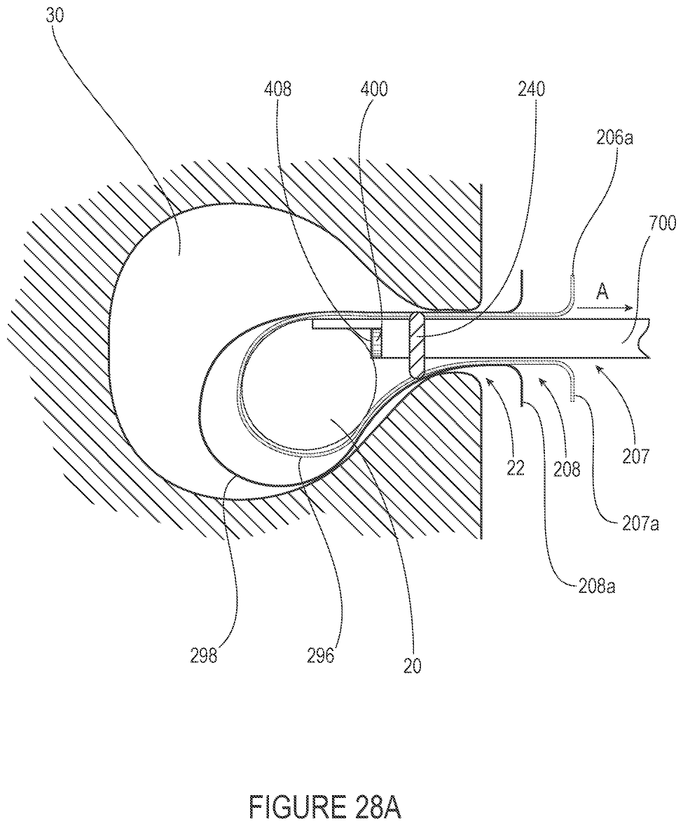

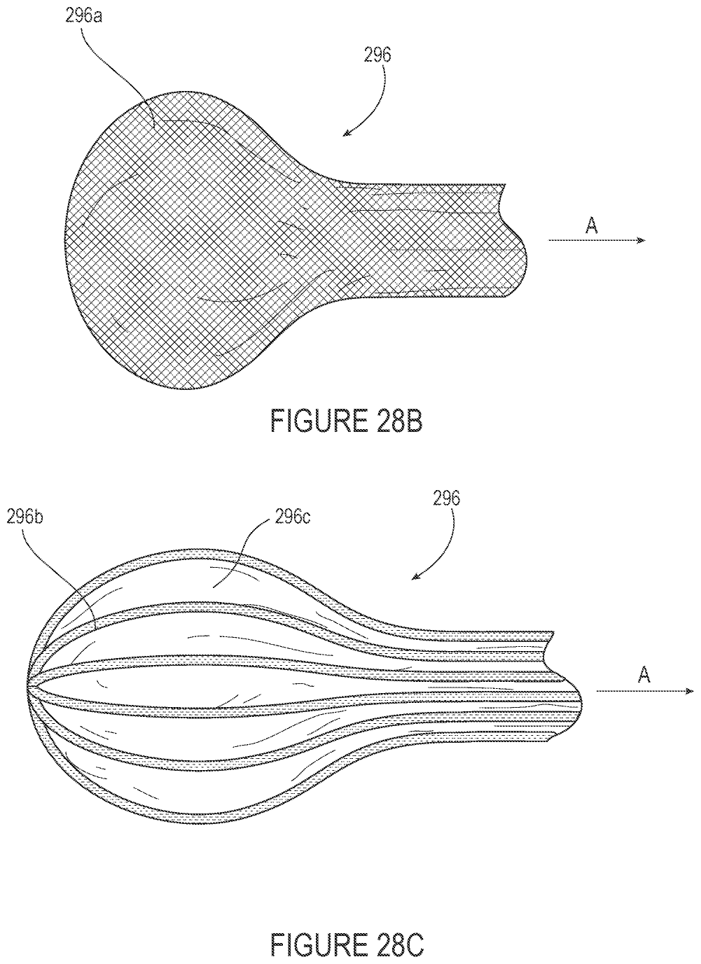

Turning to FIGS. 28A-C, system embodiment 100 employing one or more balloons 240 and two separate containers 200 is illustrated. This embodiment of system 100 may also be suitable for use without a tissue grasper, uterine manipulator, or other tool to assist in positioning the tissue specimen 20 in preparation for and during the cutting process.

FIG. 28A depicts a system 100 of the present disclosure deployed in a patient's body cavity 30, such as a pelvic or abdominal cavity, with tissue specimen 20 captured within a container 200 having two bags or containers inner bag 296 and outer bag 298. Openings 207 and 208 of each of inner and outer bags 296 and 298, respectively, are disposed outside the patient's body 31 and extending from the port (natural or surgically-created) so that inner container edge 207a and outer container edge 208a are placed as shown. Cannula 700 with cutter 400 are also shown as disposed within an interior of inner container 296, consistent with other embodiments and methods described herein. FIG. 28A also shows that inner container 296 having specimen 20 therewithin is itself disposed within an interior of outer container 298.

Cutter 400 is seen in FIG. 28A as at least partially disposed within the interior of inner bag 296. Cutter blade 408 is shown in apposition to tissue specimen 20 in a manner suggesting the beginning of the cutting process as described elsewhere herein. For example, balloon 240 is inflated in the view of FIG. 28A and in apposition against the patient's body tissue as described above in connection with methods and devices shown in FIGS. 26-27.

With a dual-bag design for container 200, specialized roles may be played by each bag and, therefore, each bag may have specific design attributes that may translate into specific properties and features. For instance, container 200 may be designed such that only inner bag 296 is in contact with specimen 20, while outer bag, which encloses inner bag 296 within its interior, may be designed to ensure bodily fluid and cellular impermeability. These differing designs may afford certain advantages in performance, cost, ease of use, and manufacturability given that a single bag, even if of a composite or bi-layer design, would by itself need to be designed to satisfy all design criteria according to embodiments disclosed herein.

Inner bag 296 may particularly be designed to have a high resistance to plastic deformation under axial loading so that the tensioning steps described herein may be safely and effectively accomplished. In particular, and as shown in FIG. 28A, openings 207 and 208 of inner and outer bags, respectively, extend out of the patient's body 31 through port or opening 22, whether that port is a surgically-created port or a naturally existing port (e.g., a vagina). In this configuration and as described elsewhere, any number of manual and automated techniques and components may be used to tension container 200 so to bring specimen 20 into apposition with or near the cutter 400 and in particular cutter blade 408, to shorten a length of inner container 296. In a simple embodiment of tensioning, a physician or other user pulls axially on a proximal end of inner container 296 by hand, optionally including a twisting motion as described elsewhere. A rolling technique, with or without rings or other components as described elsewhere herein, may be additionally utilized to apply the requisite tension in an efficient manner. Thus, a premium may be placed on an inner bag 296 having a relatively high axial strength, modulus of elasticity, and resistance to deformation under tension as described above.

Outer bag 298 may particularly be designed to have a high impermeability to fluid transfer through its walls, especially insofar as it may be impervious to leakage of cellular material (cancerous, non-cancerous, or pre-cancerous) and body fluids associated with tissue specimen 20. Given the outer bag's proximity immediately adjacent the body cavity 30 in which it is placed, outer bag 298 is the last line of defense against cavity 30 being contaminated with body fluids or tissue portions during specimen cutting and removal, thus demonstrating the relative importance of this criterion in connection with a dual-bag system 100.

In concert, inner bag 296 and outer bag 298 of this embodiment of container 200 have the attributes desired for optimal performance of a container; e.g., high fluid and tissue impermeability and high strength as described above. Other attributes, such as tear and puncture resistance, optical transmissibility, amenability to doping, use of markings such as grid/gradation lines, etc., specialized electrical or thermal properties, manufacturability, ability to be rolled, folded or compressed into a small space, etc. may also be designed into the inner and outer bags as desired.

Inner bag 296 may in fact be permeable or semi-permeable to fluids/tissues in some embodiments, while in others it may be designed to be fluid- and tissue-impermeable. In embodiments where inner bag 296 is fluid/tissue permeable or semi-permeable, it is nonetheless strong enough as described above to perform optimally during the methods described herein, including tensioning and tissue cutting and removal. For example, inner bag 296 may be constructed of a metallic material, such by a series of linkages or chains 296a (akin to a “chain mail” bag), to withstand large tensile forces and to prevent cutting by the tissue cutting device itself as illustrated in FIG. 28B. Alternatively, inner bag 296 may a composite itself, constructed of high tensile-strength strips of metallic or plastic material 296b embedded in or attached to a plastic or metallic material 296c forming the inner bag as shown in FIG. 28C. Various high-strength plastics may be used to construct or comprise such strips. Alternatively, inner bag 296 may be a single layer of material, such as isotropic PTFE and ePTFE extrusions or layered constructions in which a preferentially high tensile strength is achieved in the direction in which tensioning is to be applied, are also contemplated.

During use, system 100 as shown in FIG. 28A has been deployed as shown, with various techniques and components described herein employed to prepare and place specimen 20 within the inner bag. A tissue grasper 500 and/or laparoscopic tool may also be used to assist in placing specimen 20 in an interior of inner bag 296. Inner bag may have been prepared by being deployed within an interior of outer bag 298, either prior to the procedure as described herein or after outer bag 298 has been deployed into cavity 30 (either through a surgical or natural port). Inner bag 296 may also have been packaged within an interior of outer bag 298 so that the physician or other user may deploy both inner and outer bags as a single unit when placing container 200 into cavity 30. Once the physician or other user has pulled edges 207a and 208a of inner and outer bags out of the body port so that each of the bags' openings are outside the patient's body 31, locking member in the form of a balloon or balloons 240 may be inflated as discussed above. At this point, the physician or other user may continue apply tension on inner bag 296 by pulling in a proximal direction indicated by arrow “A” so to move tissue specimen 20 closer to the body opening 22, shortening inner bag 296. Outer bag 298 may or may not be similarly tensioned during this process depending on user preference and the particular anatomy and indication being treated.

The accomplishment of tissue specimen 20 apposition against or near cutter 400 and blade 408 may be achieved by tensioning inner bag 296 alone or alternatively tensioning outer bag 298 alone or with inner bag 296. As described above, the anchoring effect of locking member 240 in the form of a balloon as shown in place in FIG. 28A frees the physician or other operator from having to hold cannula and/or cutter during the cutting process. As tissue specimen 20 is morcellated, tension may be applied on inner bag 296 and/or optionally on outer bag 298 to maintain the proper alignment of specimen 20 and cutter 400 and to cut or morcellate specimen 20 in the desired manner. Once specimen has been processed, it may be removed from the patient's body 31, the cutter/cannula may be removed, and finally inner and outer bags 296, 298 forming container 200 separately in sequence or together may be removed from the patient's body 31. Embodiments of the systems shown in FIGS. 26-28 may include a dual-bag or a single bag container 200. In addition, it is within the scope of the present disclosure to use the dual-bag embodiment shown in FIG. 28 with or without the use of one or more locking members 240, as well as other components described herein.

In an example of a transvaginal uterine capture and removal method of the present disclosure using the system shown in FIG. 28, the inner and outer bags may be deployed into a patient's pelvic cavity either through a surgical port (not shown) in the abdominal wall or through the vagina. Once the uterus and any attendant organs/tissue has been prepared for removal, the process as described above may be employed to place the uterus into the inner bag 296, tension applied to inner bag 296 to bring the uterus near the vaginal cuff in the pelvic cavity and near or against the cutter blade 408. The cutting process may be employed to morcellate the uterus 20 and remove it through the vagina from within an interior of inner bag 296. The outer bag 298 may be removed concurrently with or after removal of inner bag, both through the vagina, and optionally one or both of inner and outer bags may be closed to keep any tissue or fluids escaping therefrom.

In another example of a transvaginal uterine capture and removal method of the present disclosure using the system of FIGS. 26-27, container 200 may be deployed into a patient's pelvic cavity either through a surgical port (not shown) in the abdominal wall or through the vagina. Once the uterus 20 and any attendant organs/tissue has been prepared for removal, the process as described above may be employed to place the uterus into container 200 and apply tension thereto to bring the uterus near the vaginal cuff in the pelvic cavity and near or against the cutter blade 408. Balloon 240 or balloons 240 may be inflated before, during or after the container tensioning step as the physician or other user sees fit. The cutting process may be employed to morcellate the uterus 20 and remove it through the vagina from within container interior 204.

Three main advantages, among others, are afforded by the systems employing one or more locking members such as the balloons 240 shown in the exemplary embodiments and methods described herein in connection with FIGS. 26-28: first, the locking member 240 may be used solely or supplementally with other guard/protector features as a guard. Second, the locking member 240 serves as an anchoring mechanism to lock or hold the cutter 400 and/or cannula 700 within which it is disposed, in place. Third, the presence and use of one or more locking members 240, such as balloons 240, supplements or even replaces the function of a tissue grasper 500 to impart counter-tension against cutter 400 during methods described herein as well as application of tension on container 200, with or without separate tensioning member or members, to pull the tissue specimen 20 against the cutter edge 408. Of course, it is within the scope of the present disclosure that systems employing one or more locking members, such as balloons 240, may be used in conjunction with components such as grasper 500, guard 300, protector portions 774, 775 and the like. It is also within the scope of the present disclosure that methods employing systems having one or more locking members, such as balloons 240, may use various techniques and tools as described herein to effect safe and proper capture, apposition, and processing of tissue specimen 20. However, the aforementioned advantages of locking members, together with other advantages such as cost reduction, ease of use, reduced complication risk, etc., make the use of such systems without additional components or techniques attractive.

In all of the examples described herein with respect to an inner and outer bag embodiment, as well as with other embodiments, the bags may be designed and employed such that no tissue grabber or other tool is necessary to achieve the methods undertaken to capture and remove tissue, thus simplifying the method as an additional tool and method steps need not be utilized.

In the example methods described above in connection with FIGS. 23-28 as well as other figures detailed in the present disclosure, certain details have been omitted for clarity. For example, proximal portions of the various components herein, including handles, triggers, hand cranks, motors, stops to control the depth of cutter blade 408, foot pedals and the like are understood variously to be available to the physician or other user when accomplishing the methods of the present disclosure, including those described below. In addition, while these methods may include use of a cannula 700 in connection with the use of cutter 400 and grasper 500, use of such cannula is optional and the methods of the present disclosure need not include such a cannula. In addition, guard 300 or protector elements such as elements 746, 778, extension 776, etc. may be integrated with or attached to cutter 400, or even tissue grasper 500, rather than cannula 700. The function of tissue grasper or tenaculum 500 may be accomplished through components or features on cannula 700 and/or cutter 400, and so a separate grasper or tenaculum 500 is also optional and need not be present to accomplish the methods described herein. For example, if the physician or other operator places enough tension on container 200 using the systems and methods discussed herein, there may be no need for a tissue grasper or tenaculum 500: the physician or other user can simply apply such axial tension on enclosure 200 to bring the tissue specimen 20 in the vicinity of or in apposition to cutter blade 408 to suffice for adequate cutting or morcellating and removal of specimen 20 as described herein.

The discussion above is directed to a variety of devices and methods for containing and removing tissue and associated procedures and devices. Much of the above discussion is included in commonly owned PCT application number PCT/US2017/029162 which was filed Apr. 24, 2017, naming S. Kim et al. as inventors and titled Systems and Methods for Tissue Capture and Removal, and which is incorporated by reference herein in its entirety. Additional device and method embodiments for containing and removing tissue and associated procedures and devices are discussed below and these additional embodiments may include aspects which are similar to the embodiments discussed above. As such, any of the embodiments discussed herein, or components thereof, may include features, dimensions and materials that are the same as or similar to features, dimensions and materials of any other appropriate or similar embodiment discussed herein.

In some cases it may be desirable to provide space within an interior volume of a container, such as container embodiments 200 discussed above, so as to allow a tissue specimen 20 disposed therein to move freely within the interior volume during tissue morcellation or at any other appropriate time during a procedure. In order to achieve such freedom of motion of a contained tissue specimen 20, space between the contained tissue specimen 20 and an interior surface of the associated container embodiments discussed herein may be achieved by a variety of techniques and devices discussed herein.

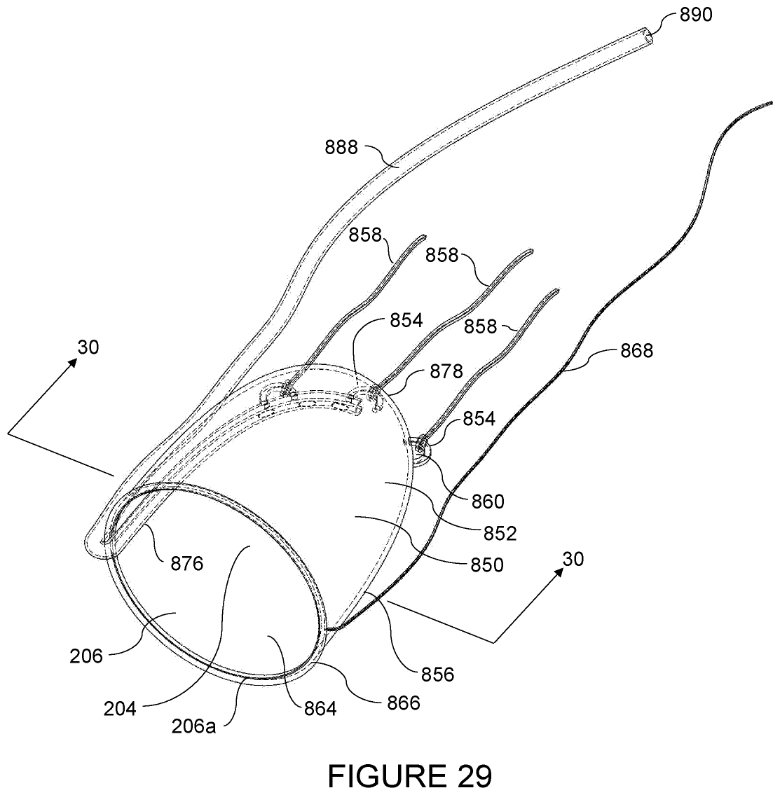

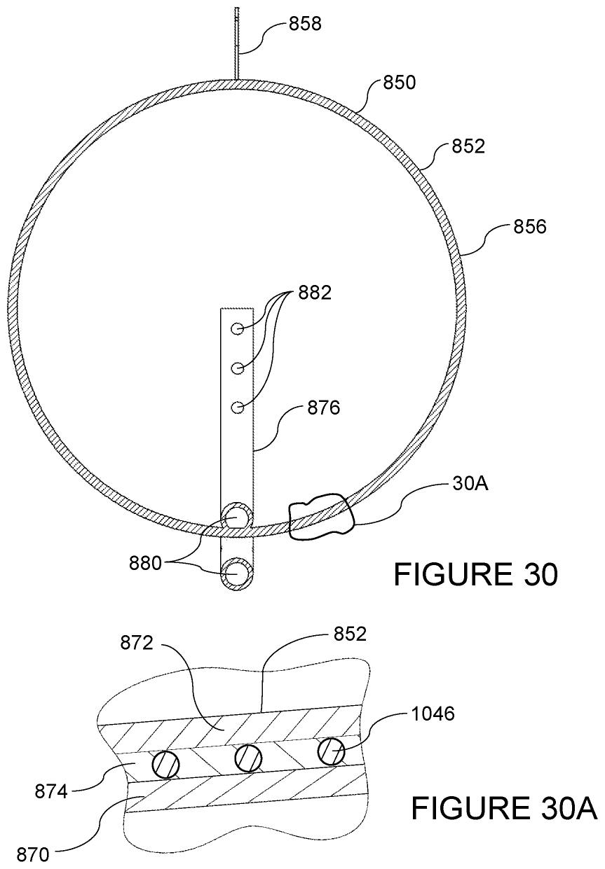

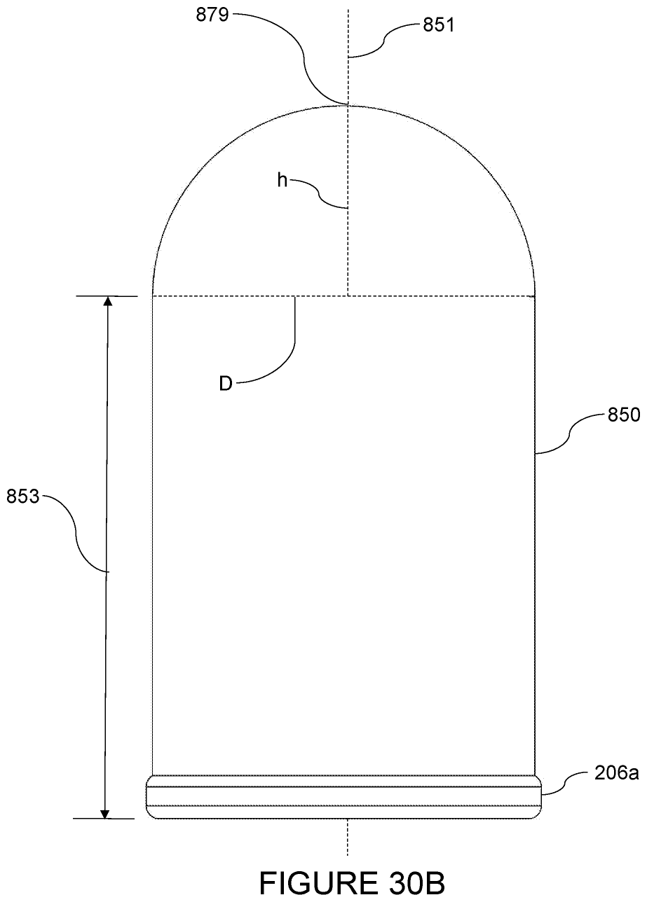

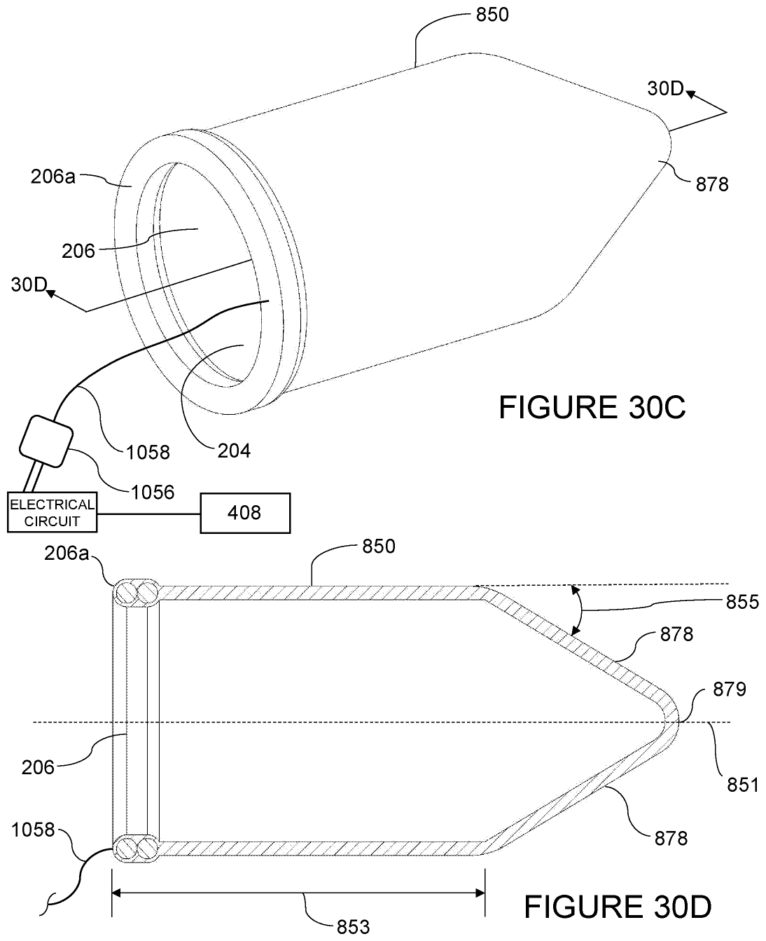

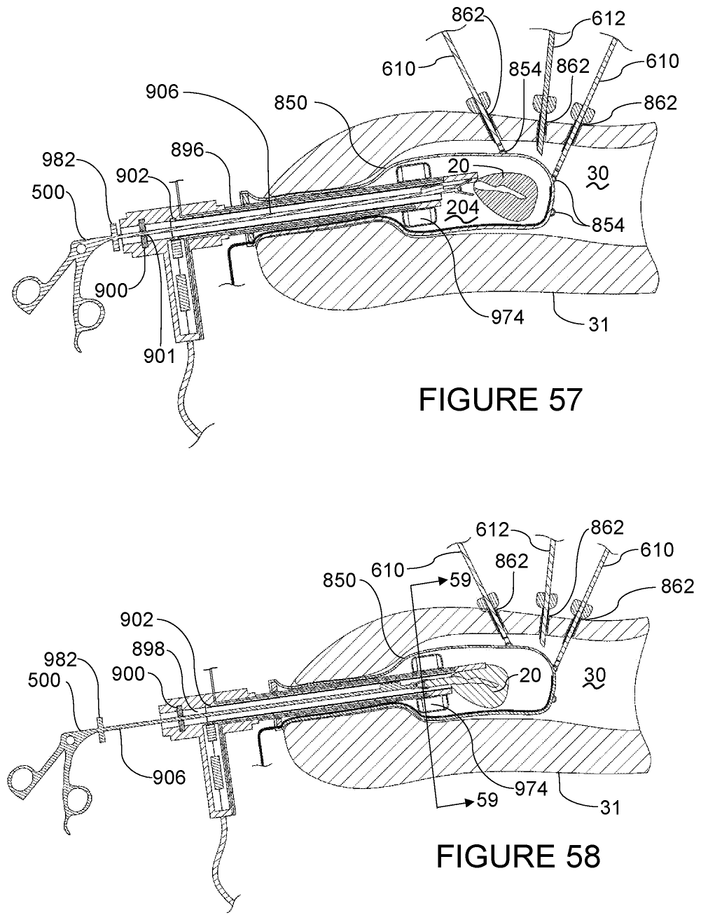

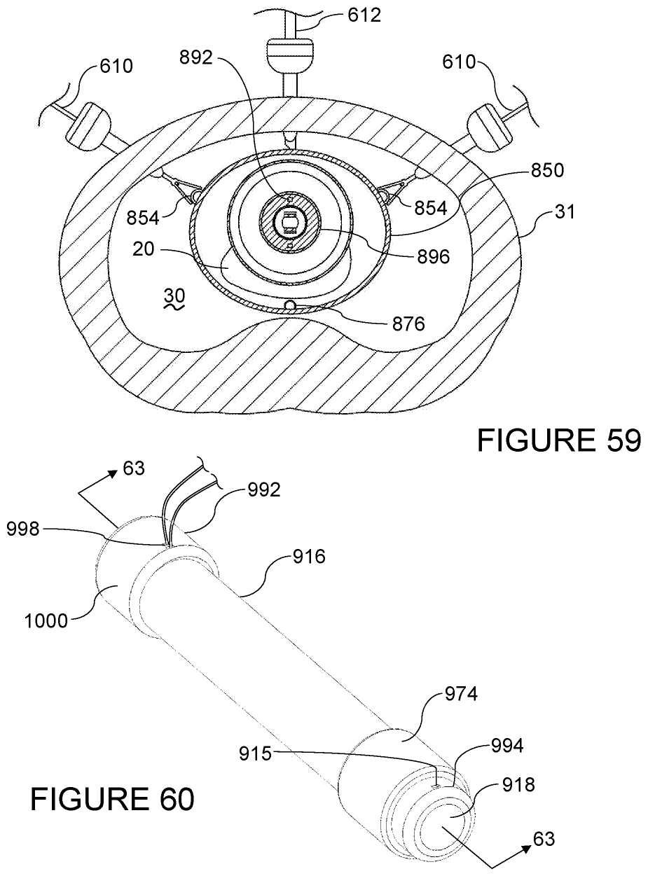

In some cases, space within a container, such as container 850 shown in FIGS. 29-30, disposed between the tissue specimen 20 and the wall of the container 850 may be achieved by applying outwardly directed tension to one or more positions on a wall of a container 850. Such a process is illustrated generally in FIGS. 57-59 in which laparoscopic instruments 610 in the form of tissue manipulators are shown applying tension to an outside surface of container 850. Containers, such as container embodiments 850 discussed herein and container embodiments 200 discussed above, may be a component of tissue containment and removal systems 100 as well as any other tissue containment and removal systems discussed herein which may optionally include tissue cutters such as tissue morcellators, cannula embodiments, access sheath embodiments, tenaculum embodiments, insufflator embodiments, as well as any other suitable components.

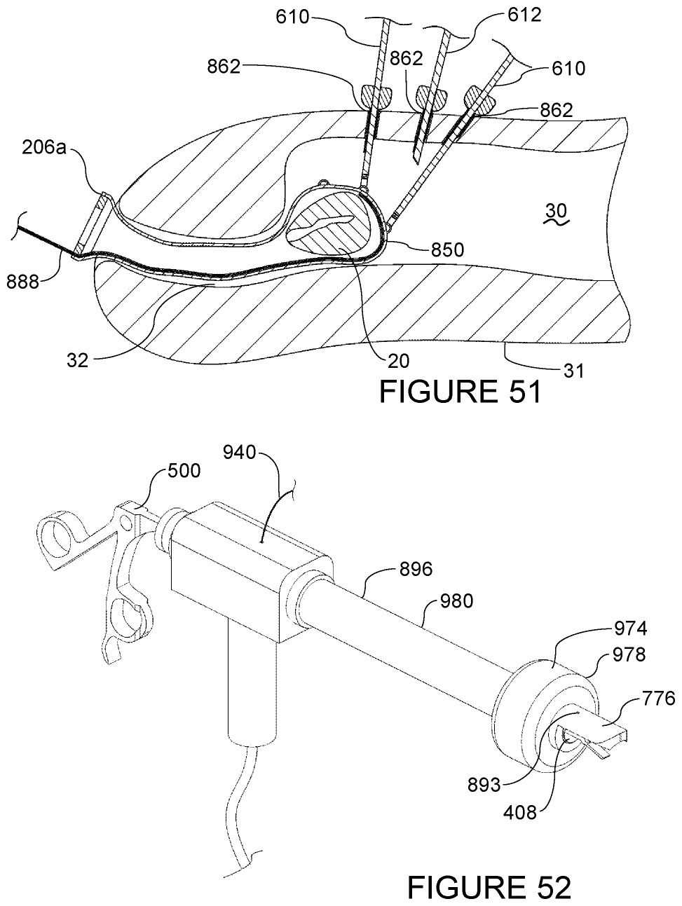

In such cases, the space between the tissue specimen 20 and wall 852 of the container 850 may be created by mechanically manipulating the wall portion 852 of the container 850 from a position outside of the container 850 in order to position the container or specifically to apply outwardly directed tension on the wall portion 852 as shown in FIG. 51. In some cases, however, directly mechanically grasping the wall portion 852 of the container 850 from a position outside of the container 850 may lead to damage of the container 850 which could in turn lead to the inability of the container 850 to remain fluid tight or isolate a contained tissue sample when pressurized. In order to avoid damage to the container wall portion 852 by surgical instruments 610 or the like, the container 850 may include a tab 854 which is secured to and extends radially outward from an outer surface 856 of the container 850 as shown in FIG. 29.

For some embodiments, the tab 854 may be integrally and monolithically formed from the material of the wall portion 852 of the container 850. For some other embodiments the tab 854 may be formed separately from the wall portion 852, and may then be secured to the exterior surface 856 of the wall portion 852 of the container 850 by any suitable means such as thermal fusion, epoxy/adhesive bond, mechanical straps, or the like. Such container embodiments 850 may include a plurality of such tabs 854, including two, three, four, five, six or more tabs 854 which may be disposed at various desired positions on the outside of the container 850. For some embodiments a tether 858 may be suitably secured to and extend from each tab 854, with the tether 858 acting as an extension to the tab 854 allowing for the grasping of the tether 858 instead of the tab 854. Such tethers 858 may be secured to the tab 854 by tying the tether 858 through an opening 860 of the tab 854.

In some cases, the tether 858 may be configured as a strand of thin flexible material such as a suture. Some container embodiments 850 may include a plurality of tabs 854, which may in some cases be color coded in order to visually distinguish between the tabs 854 and locations of those various tabs 854 relative to the structure of the associated container 850. It should also be noted that in some cases, tether embodiments 858 that are secured to and extend from the tabs 854 may have a length sufficient to be grasped while disposed within the interior volume of the patient's body cavity 30 and subsequently withdrawn from the patient's body cavity 30 through a laparoscopic port 862 or the like. Tension on the tether may then be maintained by pulling on the tether from outside the patient's body 31.

In some cases, space disposed about the tissue specimen 20 between the tissue specimen 20 and the wall 852 of the container 850 may be achieved by applying an outwardly directed force to an inside surface or generally to the wall portion 852 of a container 850, such as by insufflating or otherwise pressurizing an interior volume 204 of the container 850 with a positive pressure relative to a pressure disposed outside of the interior volume 204. Such a process is illustrated generally in FIG. 57.

Tissue containment and removal systems that include the container embodiments 850 discussed herein may further include a morcellator which may be configured to cut and remove a tissue specimen as well as any other suitable components of such tissue containment and removal systems. The container embodiment 850 shown includes an interior volume 204 which is disposed within a container interior surface 864, and an opening 206 which incorporates a container edge 206a. For some embodiments the container edge 206a may include an elastic edge member 866 which may be secured such that it is substantially adjacent to the container edge 206a, with the elastic edge member 866 being configured to maintain the opening 206 in an expanded state in order to allow for the insertion of the tissue specimen 20 into the interior volume 204 of the container 850.

For some embodiments of the container 850, the opening 206 may be configured such that it can be secured in a fluid tight manner about devices which are inserted into the interior volume 204 of the container 850. For example the edge 206a may include a feature such as the drawstring embodiment which is depicted in FIG. 6A. The container 850 may also include a distal portion 878 which is disposed opposite to the opening 206. For some embodiments, one or more elongate edge tethers 868 may be secured to and extend from the edge 206a of the container 850 in order to facilitate positioning the container 850 about a tissue specimen 20 and withdrawing the edge 206a and opening through a body conduit 22 of the patient 31.

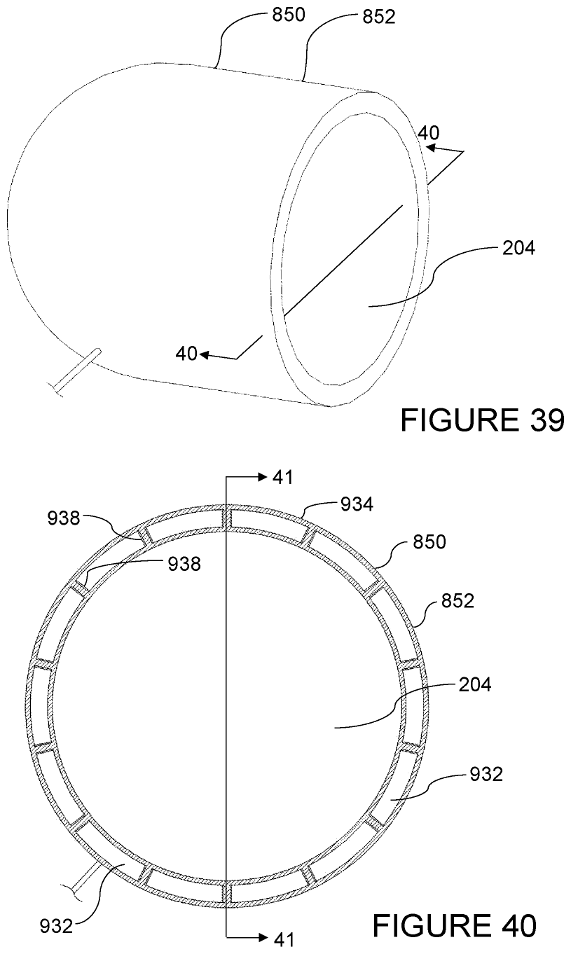

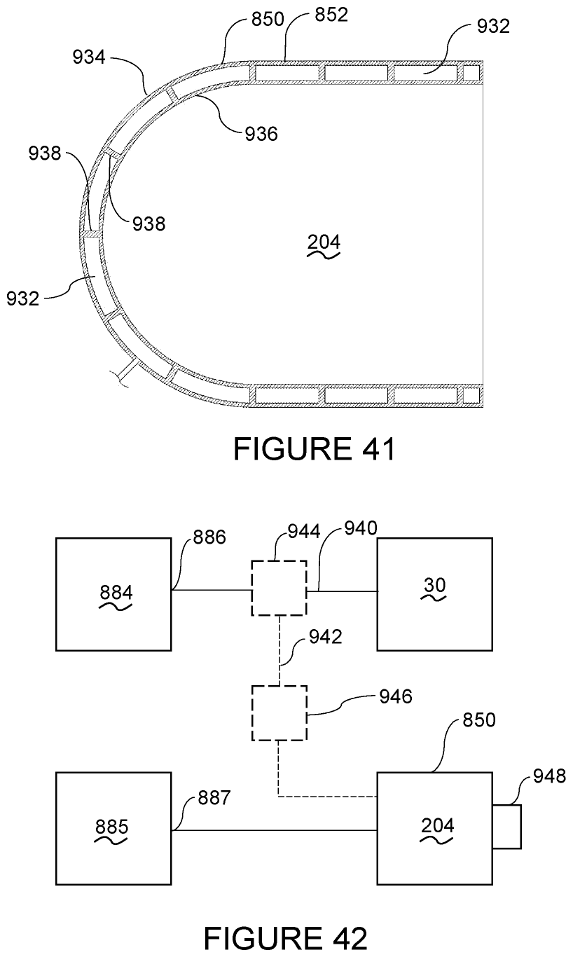

The container 850 may include the wall portion 852 which can include a layer of thin, flexible, and fluid tight material. The wall portion 852 of the container 850 may be configured for flexibility such that it allows for an outer surface 856 of the container 850 to substantially conform to surrounding tissue surfaces such as vaginal walls, abdominal walls or the like. For some embodiments 850 the wall portion 852 may be formed from a single layer of material, for some other embodiments the wall portion 852 may be formed from multiple layers of materials which may be suitably coupled together in order to form a composite wall portion 852. In some cases the multiple layers of the composite wall portion 852 may be formed from the same material, in some other cases the multiple layers may be formed from different materials. In addition, in some cases, the multiple layers of such a multi-layer wall portion 852 may be separated from each other so as to form a hollow double layer structure as shown in the container embodiment 850 illustrated in FIGS. 39-41.

Some multiple layer embodiments of the container 850 may include a bi-layer embodiment with one layer including a watertight layer 232 that prevents the egress of body fluids or cells and a second layer 230 of high strength material that prevents tearing or puncturing of the container wall by sharp edged instruments such as tissue graspers or morcellators as discussed above with regard to container embodiment 200 shown in FIG. 4. Some container embodiments 850 may include a multiple layer wall construction that includes an inner layer 870 of watertight polymer material such as polyurethane, and an outer layer 872 of the same or similar material. This multiple layer wall 852 of the container 850 may also include a middle layer 874 as shown in the optional wall portion structure embodiment of FIG. 30A wherein the middle layer 874 is disposed between the inner layer 870 and outer layer 872. Such a middle layer 874 may be made of a high strength material that resists puncturing or tearing due to contact with sharp instruments such as tenaculum tissue graspers 500, scalpels, morcellator blades 408 or the like. In some cases, the middle layer 874 may include a metallic wire mesh that has a thickness of about 0.0005 inches to about 0.004 inches, more specifically, about 0.0008 inches to about 0.002 inches, and even more specifically, about 0.001 inches to about 0.0014 inches. For some embodiments, the inner layer may include a polymer layer having a thickness of about 0.001 inches to about 0.01 inches, more specifically, about 0.0025 inches to about 0.0035 inches, and even more specifically, about 0.0028 inches to about 0.0032 inches. The middle layer may also include a same or similar configuration as that of any of the reinforcement member embodiments 252, wire filaments or beam embodiments discussed above or stiffener embodiments discussed below in some cases.

In an open substantially expanded state, some such container embodiments 850 may include an opening 206 having a transverse dimension of about 14 cm to about 16 cm and an overall axial length of about 35 cm to about 45 cm. Some such container embodiments 850 may also include an opening 206 having a transverse dimension of about 17 cm to about 21 cm and an overall axial length of about 45 cm to about 55 cm depending on the size of the tissue specimen 20 being contained and removed from the body cavity 30 of the patient 31. The wall portion 852 of such container embodiments 850 are typically flexible enough to contort and conform to irregularly shaped surfaces disposed within various embodiments of the patient's body cavity 30. In addition, although container embodiment 850 may optionally include mechanisms directed to promoting outward expansion of the wall portion 852, container embodiments 850 may also readily be used without such expansion and may be used in procedures wherein tension is applied to the edge 206a in order to reduce interior volume 204 of the container 850.

In some instances, it may further be desirable for container embodiments 850, or any other container embodiments discussed herein to include a distal portion, such as distal portion 878, that tapers distally to a reduced transverse dimension. This may be useful during tissue morcellation because as the size of the tissue specimen 20 is reduced and/or reduced to smaller pieces during the morcellation process, these smaller pieces may become displaced into corners of the container 850 that can not be readily accessed by the cutting blade 408 of the morcellator 896 even if tension is applied to the proximal portion or edge 206a of the container as discussed herein. By reducing the transverse dimension of the distal portion 878 of the container, the tissue specimen 20 and any remaining fragments thereof, will be funneled into a reduced volume portion of the interior volume 204 at the center of the container 850 thus making them accessible to the cutting blade 408 of the morcellator 896.

For example, container embodiment 850 shown in FIG. 29 has a substantially rounded distal portion 878 which tapers along a curvilinear contour to a reduced volume section towards the apex of the contour. FIG. 30B shows and embodiment of container 850 in a flattened configuration with a transverse dimension D disposed across a segment of the container 850 in an orientation which is substantially perpendicular to a longitudinal axis 851 of the container 850 in an axial position which is a distal most position of a straight section 853 of the container 850. For some such embodiments, the distal portion 878 may generally have a smooth continuous curve with a desired ratio between the transverse dimension D and the height h of the distal portion 878. The height h of the distal portion 878 is the distance between the transverse dimension line D at the distal end of the straight section 853 to the apex 879 of the distal portion 878. Some such container embodiments 850 may include a relationship between D and h wherein h is equal to about D/8 to about 2 times D, more specifically, wherein h is equal to about D/4 to about D, and even more specifically, where h equal about D/2. In some cases, it may be desirable to approximate the smooth continuous curve of the distal portion 878 as shown with a faceted distal portion (not shown) that instead includes 5 segments to about 15 segments or more.

For some container embodiments 850 having a reduced volume distal portion 878, the distal portion 878 may have a tapered or cone-shaped contour which may be centered along the longitudinal axis 851 that is angled at a desired angle. For example, referring to FIGS. 30C and 30D, container embodiment 850 is shown having an angle 855 formed between the straight section 853 which may be substantially parallel to the longitudinal axis 851. For some container embodiments, the distal portion 878 may have an angle 855 of about 5 degrees to about 85 degrees, more specifically, about 20 degrees to about 70 degrees, even more specifically, about 30 degrees to about 60 degrees, and even more specifically, about 40 degrees to about 50 degrees.

The container embodiment 850 of FIGS. 29-30D may also include an optional insufflation manifold 876 which may extend along the wall portion 852 of the container 850 from the edge 206a into the interior volume 204 towards a distal portion 878 of the container 850. The insufflation manifold 876 may also include an insufflation manifold lumen 880 which is disposed within the insufflation manifold 876 and which is configured to allow the passage of a biocompatible pressurized gas such as carbon dioxide or the like. The insufflation manifold 876 may also include at least one manifold port 882, which may be configured such that it is in fluid communication between the insufflation manifold lumen 880 and the interior volume 204 of the container 850. In some cases, the insufflation manifold 876 may include two, three, four, five, six or more manifold ports 882. For some embodiments, the insufflation manifold 876 may be integrally and monolithically formed into the wall portion 852 material of the container 850.

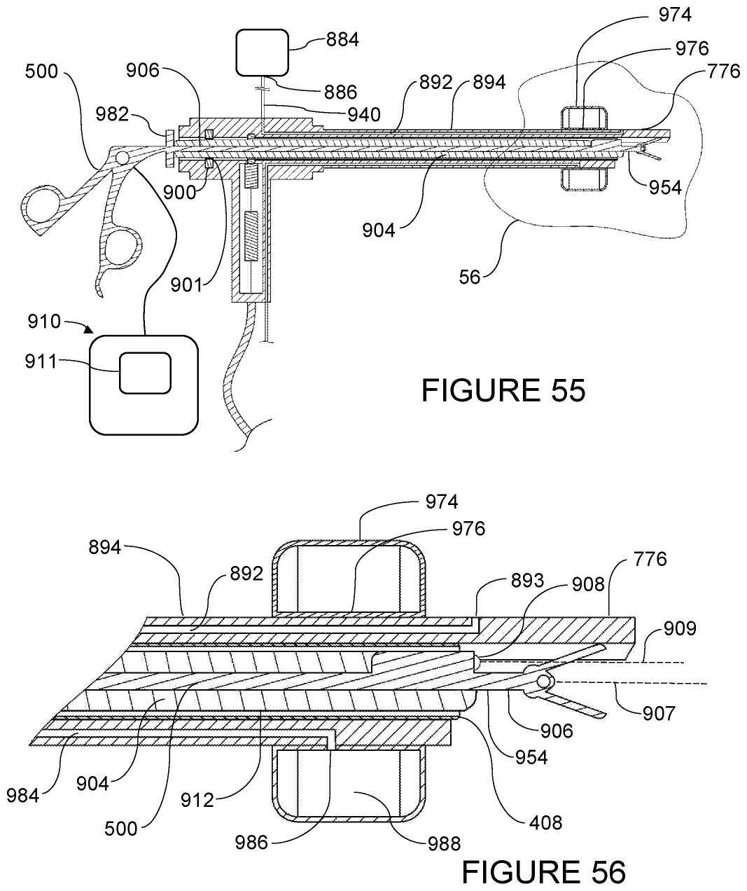

For some other embodiments the insufflation manifold 876 may be formed separately from the wall portion 852, and may then be secured to a surface such as the interior surface 864 of the container 850 by any suitable means such as thermal fusion, epoxy/adhesive bond, mechanical straps, or the like. It should be noted that the insufflation manifold 876 may be formed by being secured to an outside surface 856 of the container wall portion 852 with manifold ports 882 being disposed through adjacent wall portions 852 of the container 850 which are in fluid communication with the insufflation manifold lumen 880. Tissue containment and removal system embodiments may further include a source of pressurized gas 884, as shown in FIGS. 42 and 55, which may include an insufflator 884 having an output port 886. An insufflation tube 888 that includes an inner lumen 890 may extend from the insufflation manifold 876 and may be operatively coupled to the insufflator 884 with the output port 886 in fluid communication with the inner lumen 890 of the insufflation tube 888. In turn, the inner lumen 890 of the insufflation tube 888 may be in fluid communication with the insufflation manifold lumen 880.

In addition to providing insufflation of pressurized gas through the insufflation manifold 876, such pressurized gas may be supplied through an insufflation lumen 892 which is disposed within and extends along a morcellator cannula 894 of a morcellator 896 as shown in FIGS. 55-56. For morcellator embodiments 896 that accommodate the use of a tenaculum 500 through a central lumen 898 of the morcellator cannula 894 of the morcellator 896, one or more seals 900, 902 may be disposed within the central lumen 898 of the morcellator cannula 894 in order to provide a fluid seal between an outside surface of the tenaculum 500 and an inside surface of the central lumen 898 which is also illustrated in FIG. 55. In some instances, an optional tenaculum sleeve 904 may be disposed about an elongate shaft 906 of the tenaculum 500 from a distal portion to a proximal portion of the elongate shaft 906 of the tenaculum 500 as also shown in FIG. 55.

The tenaculum sleeve 904 may be an elongate cylindrical structure that may serve to house one or more conduits associated with the transmission of optical imaging signals from an optical objective 908 of a camera or video system 910 which is disposed on a distal end of the tenaculum 500 as shown in FIG. 56. The tenaculum sleeve 904 may also serve to provide a smooth continuous outer surface 912 having a substantially round transverse cross section with a constant transverse dimension along an axial length thereof. This smooth continuous outer surface 912 with a round transverse cross section shape may provide a consistent contour to pass through the one or more seals 900, 902 of the central lumen 898 of the morcellator assembly 896 to provide a better seal to maintain the pressure of the pressurized gas within the container 850 and associated structures.

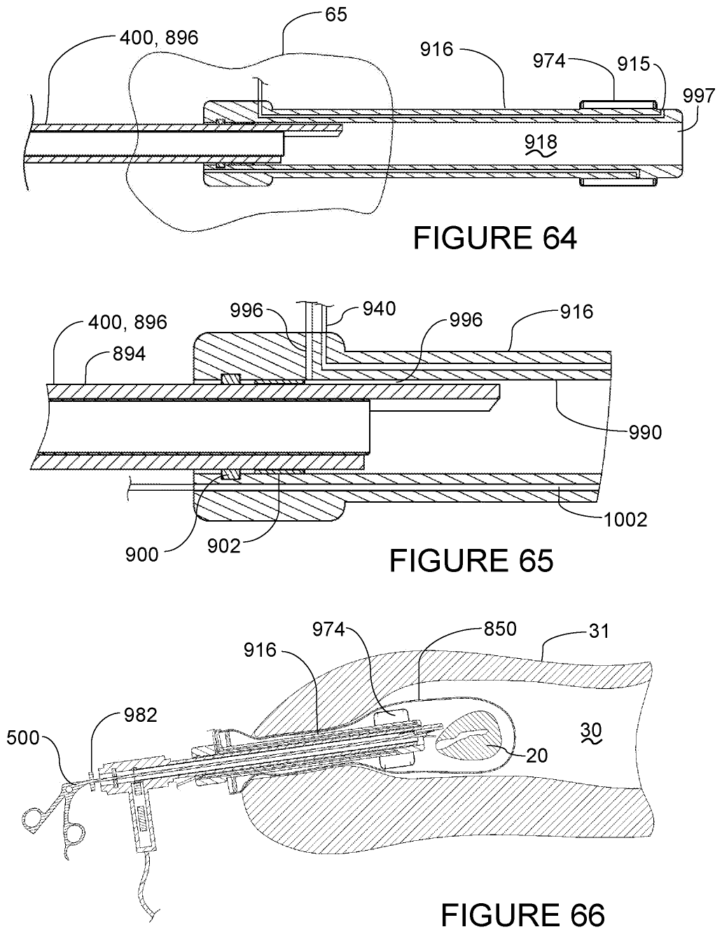

Such pressurized gas may also be supplied to the interior volume 204 of the container 850 through an insufflation lumen 914 disposed within and extending along an access sheath embodiment 916 that may be configured to facilitate access of a morcellator assembly 400, 896 or similar device into an interior volume 204 of container embodiments 850 as shown in FIGS. 63-66. In some cases, one, two or more seals 900, 902 made from a compliant high temperature material such as silicone may be used to avoid tearing. Some such seal embodiments 900, 902 may have a small centrally located hole in the seal 900, 902 that is configured to expand over an outer profile of the tenaculum shaft 906 without tearing. Also, some such seal embodiments 900, 902 may have a “duck bill” or slit type valve made from silicone or any other suitable flexible resilient material as seen, for example, in FIG. 55 which shows two seal embodiments 900, 902 disposed within a central lumen 898 of a morcellator cannula embodiment 894 and FIG. 63 which shows two seal embodiments 900, 902 disposed within a central lumen 918 of the access sheath embodiment 916.

In some instances, for method and device embodiments that utilize insufflation, it may be desirable to use insufflation lumens 880, 892, 914 that have a large oval or flattened transverse cross section in order to prevent flow restriction issues that might be problematic for sensitive commercial insufflators 884. Such oval or flattened insufflation lumens 880, 892, 914 may also be less susceptible to physical compression or occlusion when placed transvaginally or in other confined bodily access ports 22 where space is limited.

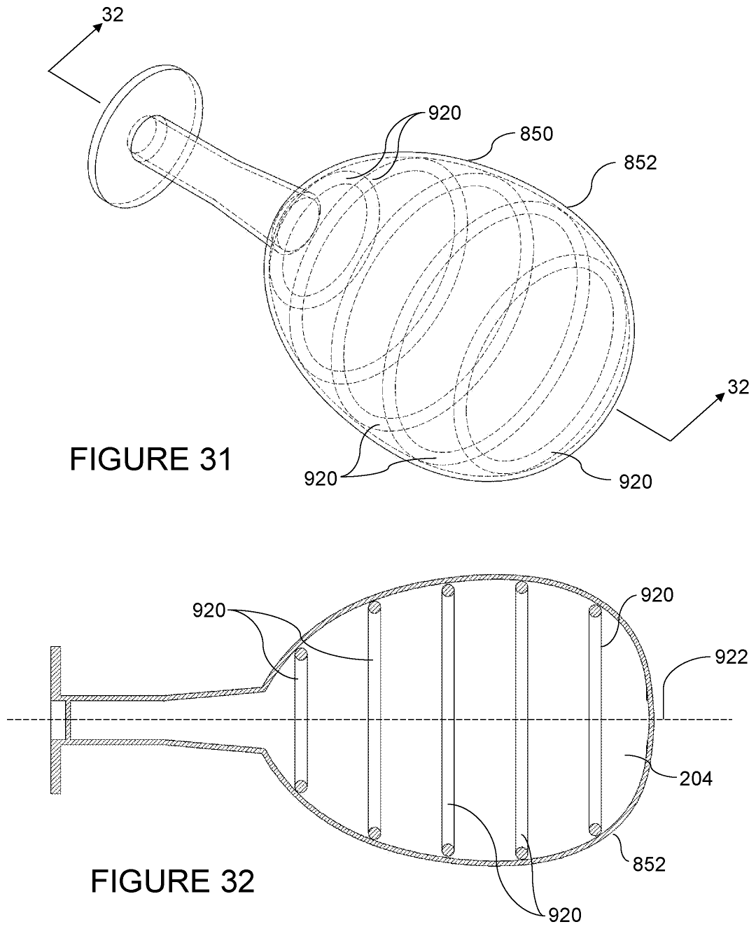

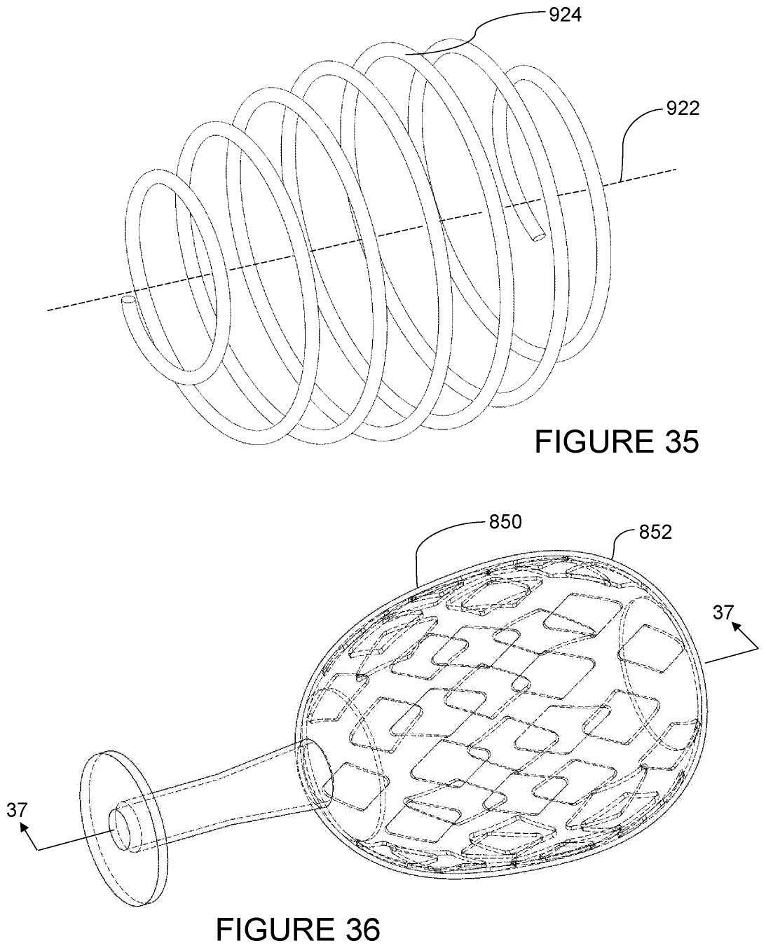

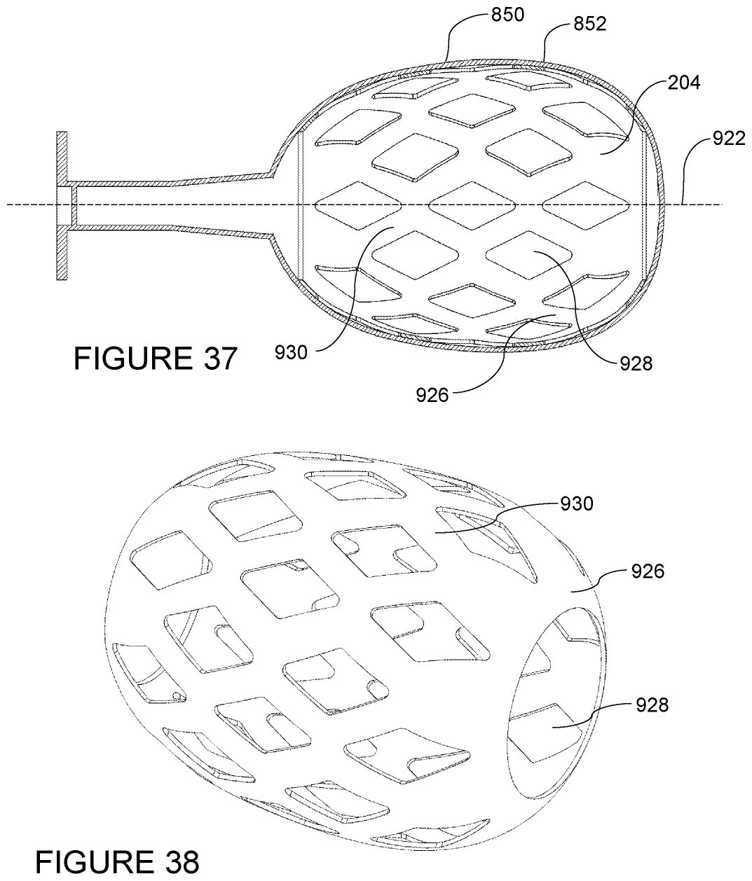

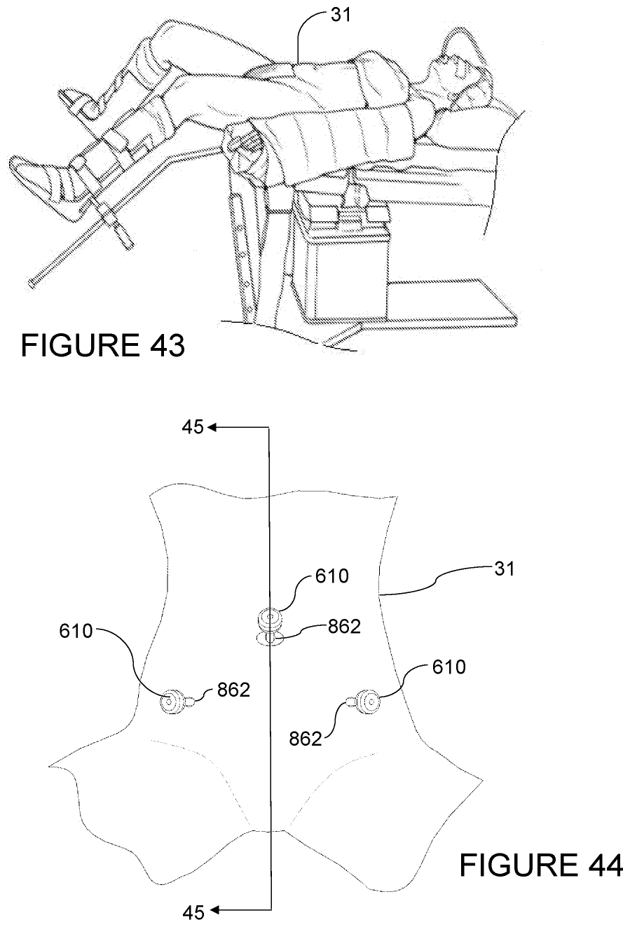

Outwardly directed forces on the wall portion 852 some container embodiments 850 in order to optionally provide working space for a tissue specimen 20 may also be achieved in some cases by the use of resilient members in the form of stiffeners which are mechanically coupled to the wall portion 852 and which have a shape and configuration that is predisposed to an enlarged volume. Several such optional stiffener embodiments are illustrated generally in the container embodiments 850 shown in FIGS. 31-38. For some embodiments, the stiffeners may include rubber or rubber-like rings, stent-like structures, cage structures, lattice structures or the like that are used to prop container embodiments open once deployed within a patient's body cavity 30 (possible patient and instrument positions are shown in FIGS. 43 and 44). Such stiffeners may be resiliently elastic and configured to collapse down to a reduced transverse dimension so as to allow passage through a restricted body access conduit 22 such as the vagina 32 and then self-expand back towards an original shape after such constraint is released.

FIGS. 31 and 32 illustrate a container embodiment 850 that includes a series of multiple ring shaped stiffener embodiments 920 which are concentrically arranged with each other along a common longitudinal axis 922 of the container 850. The stiffener embodiments 920 may be secured to the wall portion of the container 850 by any suitable means such as adhesive bonding, welding, mechanical capture within a pocket or slot of the wall portion 852, lamination between multiple layers of the wall portion 852 as shown in FIG. 30A or any other suitable means. The stiffener embodiments 920 may be made of any suitable resilient material or materials such as spring steel, NiTi alloys including superelastic alloys, polymers, composite materials or the like. In some cases, the hoop elements of the stiffeners 920 may have a transverse thickness of about 0.01 mm to about 2 mm.

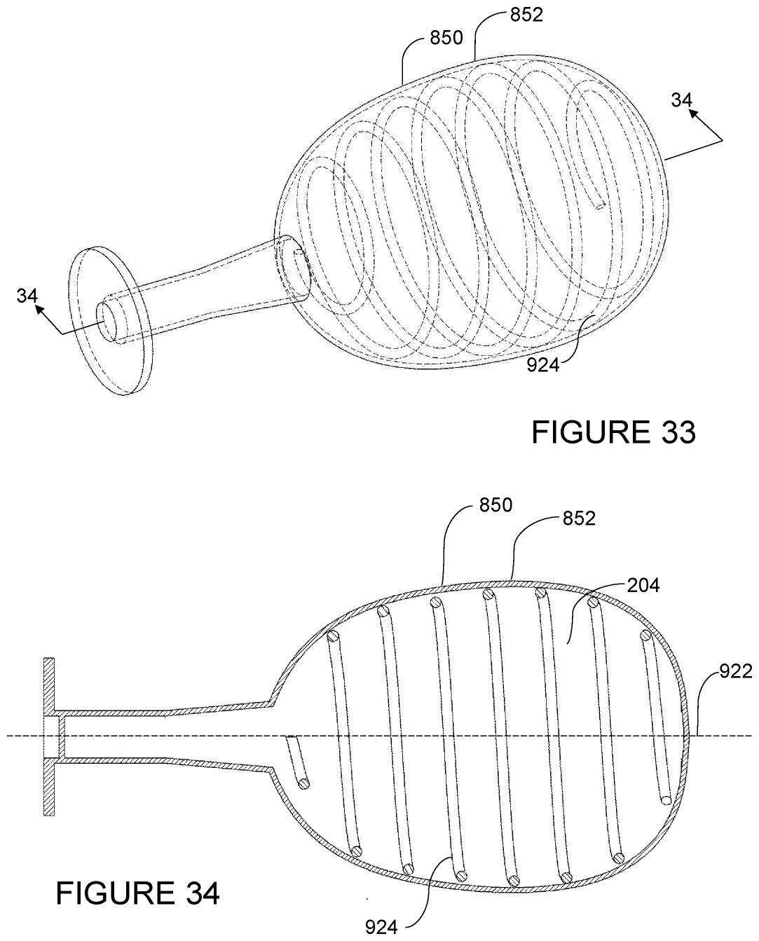

FIGS. 33-35 illustrate a container embodiment 850 that includes a single spiral shaped stiffener embodiment 924 which concentrically spirals about longitudinal axis 922 of the container 850. The stiffener embodiment 924 may be secured to the wall portion 852 of the container 850 by any suitable means such as adhesive bonding, welding, mechanical capture within a pocket or slot of the wall portion 852, lamination between multiple layers of the wall portion 852 as shown in FIG. 30A or any other suitable means. The stiffener embodiment 924 may be made of any suitable resilient material or materials such as spring steel, NiTi alloys including superelastic alloys, polymers, composite materials or the like. In some cases, the spiral element of the stiffener 924 may have a transverse thickness of about 0.01 mm to about 2 mm.

FIGS. 36-38 illustrate a container embodiment 850 that includes a single stiffener embodiment 926 having a lattice configuration which is concentrically disposed about longitudinal axis 922 of the container 850. The stiffener embodiment 926 has a shell structure with a plurality of apertures 928 and struts 30 disposed about the apertures 928. The stiffener 926 may be secured to the wall portion 852 of the container 850 by any suitable means such as adhesive bonding, welding, mechanical capture within a pocket or slot of the wall portion 852, lamination between multiple layers of the wall portion 852 as shown in FIG. 30A or any other suitable means. The stiffener embodiment 926 may be made of any suitable resilient material or materials such as spring steel, NiTi alloys including superelastic alloys, polymers, composite materials or the like. In some cases, the shell structure of the stiffener 926 may have a transverse thickness of about 0.01 mm to about 2 mm.

Shape control on some container embodiments 850 in order to provide optional working space within an interior volume 204 of the container 850 for a tissue specimen 20 may also be achieved in some cases by the use of a double walled container 850 as shown in FIGS. 39-41. For such embodiments 850, pressurized gas may be introduced into an interior volume 932 disposed between an outside layer 934 and an inside layer 936 of the double walled container 850 in order to provide a resilience to the wall portion 852 of the container 850 that tends to an expanded open state upon inflation of the interior volume 932. For such embodiments, it may be desirable to use one or more tendons 938 secured between the outside layer 934 and inside layer 936 of the double walled container 850 in order to maintain a separation between the two layers 934, 936 substantially at a predetermined distance and to maintain the two layers 934, 936 substantially parallel to each other in some cases.

The outer layer 934 and inside layer 936 of the wall portion 852 may be configured to be separated from and substantially parallel to each other and held in relative approximation to each other by the plurality of tendons 938 which may have a thin flexible structure. Each tendon 938 may serve to fix the relative separation of juxtaposed portions of the respective outside layer 934 and inside layer 936 of wall portions 852 adjacent each tendon 938. Such a configuration may be used such that an interior volume 204 of the container 850 may be maintained at a low pressure, such as atmospheric pressure, relative to the pressure within the interior volume 932 disposed between the two wall layers 934, 936 of the container 850. For some embodiments, the interior volume 932 between the two wall layers 934, 936 may be pressurized to about 15 mmHg and the interior volume of the body cavity 30 within which such a container embodiment 850 is disposed may be maintained at a pressure of about 15 mmHg or less. The container embodiment 850 shown in FIGS. 39-41 may include any of the suitable features, dimensions or materials as those of any other container embodiments 200, 850 discussed herein.

Additionally, such shape control and stiffening of a wall portion 852 of container embodiments 850 discussed herein may be achieved in some cases with the use a thick walled container which resists collapse under tension or external pressure from the pressurized gas emitted from insufflator embodiments 884. For some embodiments, containers 850 having such stiffness may have a wall thickness of about 0.005 inches to about 0.03 inches.

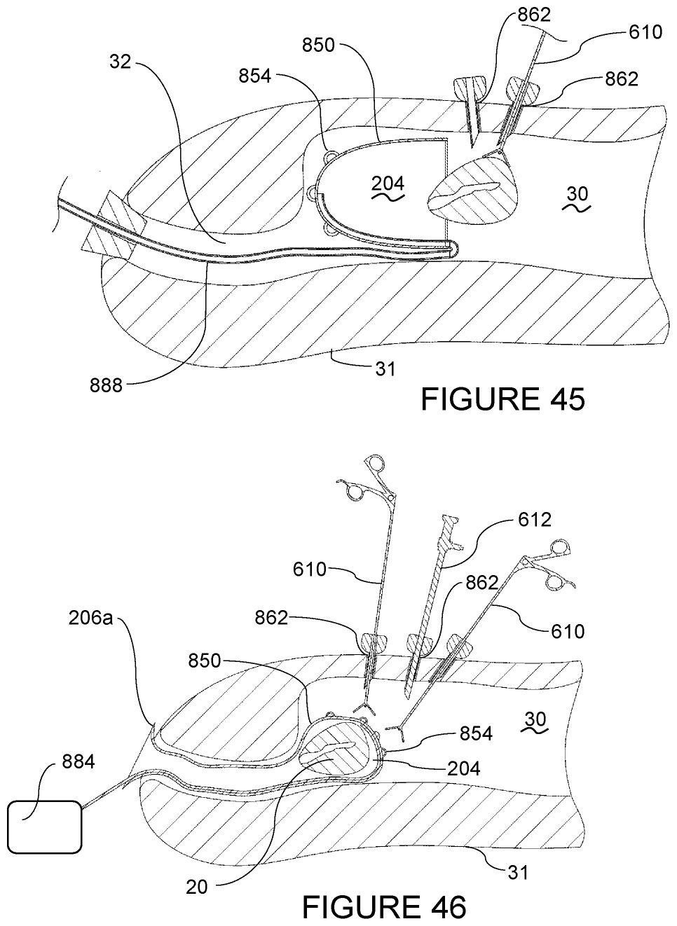

Method and device configurations for optional relative inflation of the body cavity 30 and interior volume 204 of various container embodiments 200, 850 discussed herein and as shown in the exemplary method embodiments illustrated in FIGS. 45-51, 57-59, and 62-66 may vary depending on the indication and systems being used. For example, one way to insufflate an interior volume 204 of a container 850 and interior volume of a body cavity 30 may include the use of two insufflators. For such an embodiment, the interior volume of the body cavity 30 and internal volume 204 of the respective container 850 may each have a separate dedicated insufflator in fluid communication therewith, as shown by the conduits represented by the solid lines in the schematic diagram of FIG. 42. More specifically, for such an embodiment, the interior volume of the patient's body cavity 30 may be in fluid communication with an output port 886 of a first insufflator 884 and an internal volume 204 of an exemplary container 850 may be in fluid communication with an output port 887 of a second insufflator embodiment 885. In this way, the internal pressure of each internal volume 204, 30 may be set at a desired level relative to the surrounding environment and relative to each other. For some insufflation methods, the interior volume 204 of the container 850 and interior volume of the body cavity 30 may each be insufflated to a pressure of about 15 mmHg or any other suitable pressure.

In some cases, however, a single insufflator 884 may be used to pressurize both the interior volume of the body cavity 30 and interior volume 204 of a container 850. When using a single insufflator 884 to pressurize two different volumes, a variety of methods may be used. For example, the pressure in each respective interior volume 30, 204 could be maintained at equal pressures by putting the insufflator 884 in direct fluid communication with each of the respective interior volumes 30, 204 by use of a split “Y” connector or the like. Alternatively, a pressure regulator T-valve 944 may be disposed in fluid communication between a second insufflation tube 942 which is in fluid communication with the interior volume204 of the container 850 and a first insufflation tube 940 which is in fluid communication with the interior volume of the body cavity 30 (or between the two interior volumes 30, 204 directly). In this method, the insufflation pressure within the interior volume 204 of the container 850 may be maintained at a higher pressure than the insufflation pressure within the body cavity 30 outside of the container 850. A schematic diagram of such an arrangement is shown in FIG. 42 corresponding to the embodiment that includes the dashed lines and a single first insufflator 884 only.

More specifically, referring to the dashed line structures of FIG. 42, some embodiments of tissue containment and removal systems 100 may include a container 850 which is configured to contain and isolate a tissue specimen 20. The container 850 (which is shown schematically in FIG. 42) may include an interior volume 204 and an opening 206 having an edge 206a. The container 850 may also include a distal portion 878 which is disposed opposite the opening 206, and a wall portion 852 which may be configured such that it is thin and flexible and may be formed from a fluid tight material. The tissue containment and removal system 100 may also include one or more sources of pressurized gas (such as an insufflator 884) having an output port 886.

Such tissue containment and removal systems 100 may also include the first insufflation tube 940 having an inner lumen in fluid communication between a respective output port 886 of the first insufflator 884 and an interior volume of a patient's body cavity 30, within which the container 850 is deployed. The tissue containment and removal system 100 may also include a second insufflation tube 942 having an inner lumen in fluid communication between output port 886 and the interior volume 204 of the container 850. A pressure regulator valve 944 may be disposed in the second insufflation tube 942 between the interior volume 204 of the container 850 and the outlet port 886. The pressure regulator valve 944 may be configured to restrict a flow of pressurized gas from the second insufflation tube 942 until a predetermined threshold pressure has been reached in the interior volume 204 of the container 850.

The tissue containment and removal system 100 may also include an on/off valve 946 which is disposed in the second insufflation tube 942 between the pressure regulator valve 944 and the interior volume 204 of the container 850. For some tissue containment and removal system embodiments 100 may further include a morcellator 400, 896 which has a distal end which is at least partially disposable within the interior volume 204 of the container 850. The morcellator 400, 896 may include a morcellator cannula 894 which may have a central lumen 898 which extends an axial length of the morcellator cannula 894.

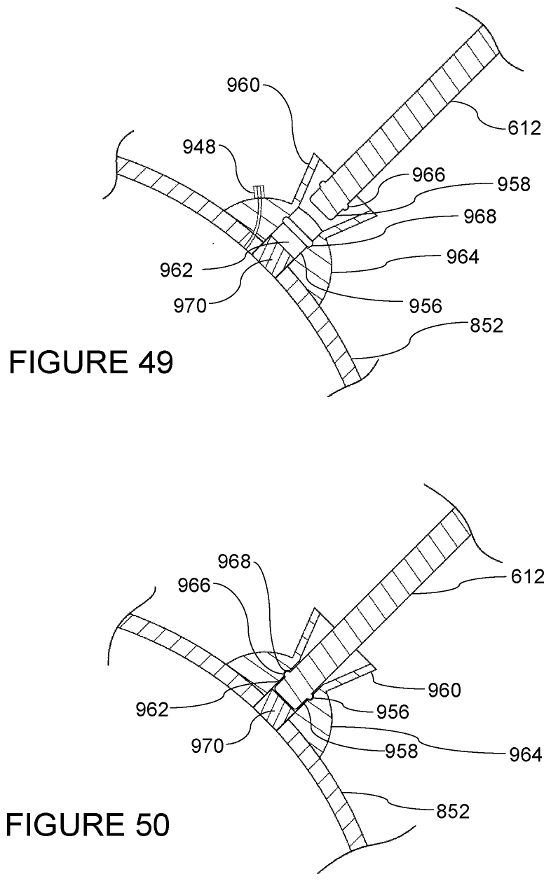

A similar result may also be achieved with an insufflator 884 being coupled solely to the interior volume 204 of a container 850 with a pressure regulator valve 948 configured to function as a “crack” valve that does not open until a predetermined pressure differential between the interior volume 204 of the container 850 and the environment disposed about the container 850 is achieved. The optional pressure regulator valve 948 may be disposed on the wall portion 852 of the container 850 as shown, for example, in FIG. 49. Such a configuration may allow fluid communication between the interior volume 204 of the container 850 and the interior volume of the body cavity 30 outside of the container when the pressure differential between these two interior volumes 30, 204 is above a predetermined value set within the pressure regulator valve 948. For such an embodiment, the interior volume 204 of the container 850 may generally insufflate first, and the interior volume of the body cavity 30 outside of the container 850 may subsequently inflate with pressurized gas exiting from the pressure regulator valve 948 once the requisite pressure differential is achieved. As such, the container 850 may insufflate first and expand to its full volume prior to insufflation of the body cavity 30 surrounding the container 850.

Again referring to FIG. 42, some tissue containment and removal system embodiments 100 may include the container 850 which is configured to contain and isolate a tissue specimen 20. The container 850 may include the interior volume 204 and an opening 206 having an edge 206a. The container 850 may also include a distal portion 878 which is disposed opposite the opening 206, and a wall portion 852 which may be configured such that it is thin and flexible and may be formed from a fluid tight material as well as other materials that serve other functions such as tear or puncture resistance. The tissue containment and removal system embodiments 100 may also include the pressure regulator valve 948 which is disposed on the wall portion 852 of the container 850.

The pressure regulator valve 948 may be disposed such that it is in fluid communication between the interior volume 204 of the container 850 and a position exterior to the container 850. The pressure regulator valve 948 may be configured to restrict a flow of pressurized gas out of the interior volume 204 of the container 850 until a predetermined threshold pressure has been reached in the interior volume 204 of the container 850. Some tissue containment and removal system embodiments 100 may further include a morcellator 400, 896 which has a distal end which is at least partially disposable within the interior volume 204 of the container 850. The morcellator 400, 896 may include a morcellator cannula 894 which may have a central lumen 898 which extends an axial length of the morcellator cannula 898.

In some cases, it may also be useful to enhance visualization of an interior volume 204 of a container 850 during a procedure in order to better control the process of tissue manipulation, tissue morcellation and tissue removal or for any other suitable tissue containment and removal procedure. As such, it may be desirable to provide optical objective elements 908 of one or more video systems or cameras 910 in a position that efficiently images the interior volume 204 of container embodiments 850. For some embodiments, such the optical objective 908 may be disposed on a distal section 954 of a tenaculum 500 with an optical axis 909 that is facing a distal direction along a longitudinal axis 907 of the tenaculum 500 generally, as shown in FIGS. 55-56. For such embodiments, a video display 911 for the tenaculum 500 mounted optical objective 908 may be disposed in any convenient location within the operating area, or may be disposed directly on a proximal portion of the tenaculum device 500 itself in some instances. Video systems 910 that include such optical objectives 908 and video displays 911 may include fiber optic scopes that include coherent fiber bundles, light sources, objective lenses and display screens or any other suitable components.

For some embodiments, visualization of the interior volume 204 of the container 850 may be enhanced by the properties of the container 850 itself. For example, some container embodiments 850 may include a wall or wall portions 852 that are transparent so as to allow visualization of an interior volume 204 of the container 850 from a position outside of such a container 850. In addition, in some instances, devices such as a laparoscopic dock 956 may be disposed on a wall portion 852 of some container embodiments 850 so as to allow optical and/or mechanical coupling between a distal end 958 and optical objective of a laparoscope 612 with a wall portion 852 of a container 850. Such a laparoscopic dock 956, as shown in FIGS. 49-50, may allow visualization of an interior volume of a container by virtue of optical coupling and mechanical contact between a distal end of the laparoscope 612 and laparoscopic dock without damage to the container due to heat emitted from the distal end 958 of the laparoscope 612.

Some such laparoscopic dock embodiments 956 may also include a cone or funnel structure 960 adjacent to and extending from a laparoscope channel 962 of the laparoscopic dock 956 in order to help guide the distal end of a laparoscope 612 into the laparoscope channel 962 so as to be mechanically coupled to the laparoscopic dock 956. The laparoscope 612 may also be coupled by a latch, detent, magnetic coupling or any other suitable device. In some instances, it may also be desirable for a laparoscope 612 to be passable into the interior volume 204 of container embodiments 850 through a self-sealing valve or port (not shown) in a wall portion 852 of the container 850.

Some tissue containment and removal system embodiments 100 may also include the laparoscope 612 as shown in FIGS. 46-50 which allows for remote visualization of tissue removal procedures. In some cases during a tissue containment and removal procedure, it may be advantageous to utilize the laparoscope 612 to image inside the interior volume 204 of the container 850. Imaging via the laparoscope 612 through the wall portion 852 of the container 850 may be impaired by poor optical properties of the material of the wall portion 852 in some cases, or by opaque structures which may be disposed within the interior volume 204 of the patient's body cavity 30 outside of the container 850. It may thus be desirable to couple the laparoscope 612 to the outside surface 856 of the container 850.

In order to achieve such coupling, some container embodiments 850 may include the laparoscope dock 956 as shown in FIGS. 49-50 which may be configured to operatively couple to the laparoscope 612 to the container 850 so as to allow for enhanced imaging into the interior volume 204 of the container 850 via the laparoscope 612. The laparoscope dock 956 may include a body portion 964 which may be secured to the wall portion 852 of the container 850. The laparoscope dock 956 includes the laparoscopic channel 962 which may be disposed within the body portion 964 and which may be sized to fit a distal end 958 of the laparoscope 612. That is to say, a diameter of the laparoscopic channel 962 may be configured such that an inner transverse dimension of the laparoscope channel 962 will adequately fit in a coupled configuration with an outer transverse dimension of a distal end 958 of the laparoscope 612 being used so as to adequately position the laparoscope 612 in both an axial and angular orientation for desired visualization of the interior volume 204 of the container 850.

For some embodiments a mechanical coupling arrangement may be used to releasably secure the distal end 958 of the laparoscope 612 to the laparoscope dock 956. For example, for some embodiments, the distal end 958 of the laparoscope 612 may include a ridge 966 which may be configured to couple or otherwise snap fit into a corresponding channel indent 968 which is disposed on an interior surface of the laparoscopic channel 962 thereby releasably securing the laparoscope 612 to the laparoscope dock 956. The laparoscope dock 956 may be formed from any suitable material and may be secured to the wall portion 852 by any suitable means such as epoxy/adhesive, or mechanical tethers.

The laparoscope dock 956 may also include an optional window 970 which is disposed at an inner end of the laparoscopic channel 962. The window 970 may be configured with optical properties which allow for enhanced imaging via the laparoscope 612 into the interior volume 204 of the container 850. For some embodiments, the window 970 may be formed from a transparent section of the wall portion 852 of the container 850. For some embodiments the window 970 may be formed from a high temperature transparent material such as glass in order to provide additional heat resistance from heat which may be generated from a distal end 958 of a laparoscope 612 being coupled to the laparoscope dock 956.

In order to assist with the coupling of a laparoscope 612 to the laparoscope dock 956, the hollow cone 960 as shown in FIGS. 49-50 may be secured to or integrally formed with the laparoscope dock 956. The hollow cone structure 960 may be secured to and may extend outwardly from the body portion 964 of the laparoscope dock 956. An interior space disposed within the hollow cone may flare to a larger transverse dimension away from the body portion 964 of the laparoscope dock 956 to form a funnel-like configuration that facilitates guiding the distal end 958 of the laparoscope 612 into the laparoscopic channel 962. For some embodiments, the distal end 958 of the laparoscope 612 may also include an optional heat shield 972 that extends distally from a light emitting surface at the distal end 958 of the laparoscope 612 as shown in FIG. 48. The heat shield 972 may prevent contact between the wall portion 852 of the container 850 and the light emitting surface at the distal end 958 of the laparoscope 612 thereby potentially preventing thermal damage to the wall portion 852 of the container 850.

During some procedures for containment and removal of a tissue specimen 20 from a body cavity 30, one, two, three, four or more laparoscopic ports 862 may be deployed in order to access an abdominal cavity 30 of a patient 31 as shown in FIG. 46. For certain procedures, such access may be useful in some instances for performing a dissection of a patient's uterus and uterine cervix from the vagina 32 for example. In such cases, the instruments 610 used to perform the dissection procedure through the laparoscopic ports 862 may have necessarily rough or even sharp edges that may pose a risk of damage to the wall portion 852 of the container 852 used to isolate and confine a tissue specimen 20 such as the patient's uterus.

Note that in some cases, even instruments 610 that have mechanically atraumatic distal ends, may still pose a damage risk to a wall 852 of a container 850. For example, some laparoscopes 612 emit large amounts of visible light from distal ends 958 thereof which may also result in a significant heating of the distal end 958 of such devices 612. This heat generated by the illumination energy of the laparoscopes 612 may also damage the wall portion 852 of some container embodiments 850. Due to the potential inconvenience of removing instruments 610 that may pose a damage risk to certain container embodiments 850, it may be preferable to provide certain techniques and mechanisms in order to prevent contact between such instruments 610 and the wall potion 852 of the container embodiments 850.

For example, as discussed above, tab embodiments 854 and tether embodiments 858 may extend from and be secured to wall portions 852 of container embodiments 850 allowing a surgical instrument 610 such as a grasper to mechanically couple to the wall portion 852 of the container 850 without creating the potential for mechanical damage to the wall 852 of the container 850 as shown in FIGS. 1 and 51 may be desirable. Once so grasped, the tabs 854 may be manipulated by the one or more graspers 610 in order to move the container wall 852 into a desired position relative to the abdominal cavity 30, tissue specimen 20, laparoscope 612 or any other structure associated with a tissue containment and removal procedure. In some cases, specifically with regard to heat generated by a distal end 958 of the laparoscope 612, it may be desirable to include the shield 972 on the distal end 958 of the laparoscope 612 in order to prevent contact of the heated distal end 958 of the laparoscope 612 with the wall portion 852 of the container 850. An example of such a heat shield embodiment 972 is shown in FIG. 48.

In addition to risk of mechanical damage from laparoscopic tools 610 introduced from laparoscopic ports 862 in the patient's abdominal wall, some container embodiments 850 may also be susceptible to mechanical damage from certain elements of morcellator 400, 896 or other cutting tool embodiments. To address this concern, a locking member 974 that has an expandable configuration may be disposed on or near a distal end 976 of the morcellator cannula 894 of some morcellator embodiments 896 as shown in FIG. 52 or access sheath embodiments 916 as shown in FIG. 60.

In some instances, such locking members 974 may include inflatable members such as an inflatable balloon 978 having a generally annular shape and being expandable from a collapsed state that generally follows the outer contour of the morcellator cannula 894 to an expanded state that extends radially outward from an outside surface 980 of the morcellator cannula 894 so as to provide an enlarged transverse section that prevents the container wall 852 from coming into contact with the blade 408 of morcellator embodiments 400, 896 and also prevents the morcellator 896 or access sheath 916 from being accidentally withdrawn from the patient's body conduit 22 during use. Such locking member embodiments 974 may also be useful for sealing the container 850 to an inside surface of the body cavity 30. In some instances, locking member embodiments 974 may have an outer transverse width in an expanded state of about 0.5 cm to about 40 cm, more specifically, about 1.5 cm to about 5 cm.

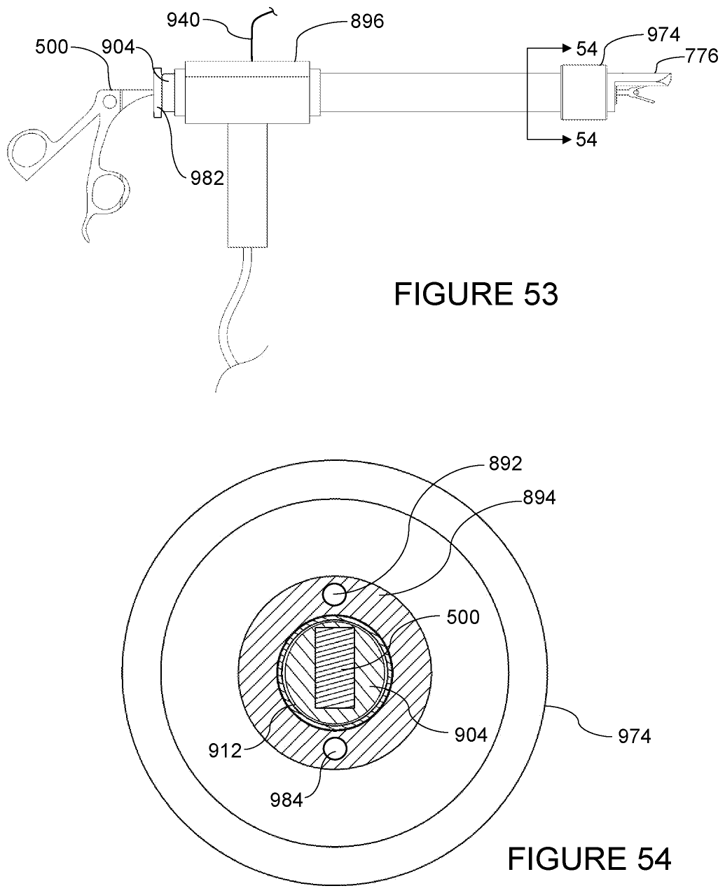

A tenaculum stop 982 as shown in FIG. 53 may also be used to prevent extension of the distal end 954 of the tenaculum 500 from the blade 408 of the morcellator 896 beyond a predetermined distance. In some cases, the tenaculum stop 982 may be located on the shaft 906 of the tenaculum 500 so as to limit extension of the distal end 954 of the tenaculum 500 beyond the blade 408 to a distance of about 1 cm to about 30 cm, more specifically, about 10 cm to about 18 cm.

Another element that may be useful for the prevention of mechanical damage to container embodiments 850 may include an extension 776 that extends distally from the distal portion 976 of the morcellator cannula 894 of the morcellator assembly 896 adjacent the blade 408 of the morcellator 896 as shown in FIG. 52. For some embodiments, the extension 776 may extend about 0.5 cm to about 40 cm from the blade 408 of the morcellator 896, more specifically, about 2 cm to about 10 cm. Such an extension 776 may also be useful for guiding the tissue specimen 20 into the blade 408 of the morcellator 896 to achieve an efficient cutting cross section of the tissue specimen 20.

The extension 776 may also be important in some cases for positioning target tissue to be cut on a tangent surface or volume of the target tissue 20 such that the blade 408 is not coring but rather peeling the target tissue 20 of the tissue specimen 20 as it is being morcellated. When target tissue 20 is in contact with solely with the extension 776, this target tissue 20 is disposed on one side of the extension 776 to facilitate a peeling type morcellation. In some cases, extension embodiments 776 may include a ramp or thickness profile such that the cut occurs on a tangent as shown in FIGS. 18B and 18C discussed above and not a full circular coring type cut.

Tissue containment and removal system embodiments 100 may also include a morcellator 896 as shown in FIG. 52 which may be configured to be inserted into the interior volume 204 of the container 850 and to cut and remove a tissue specimen 20 which is contained and isolated within the interior volume 204 of the container 850. The morcellator 896 may have a distal end 976 which may be at least partially disposable within the interior volume 204 of the container 850 and may further include the morcellator cannula 894 which has a central lumen 898 which extends through an axial length of the morcellator cannula 894. In some cases, the tissue containment and removal system may further include a tenaculum 500 which may be slidably disposed within the central lumen 898 of the morcellator cannula 894 as shown in FIG. 55.

The central lumen 898 of the morcellator cannula 894 may further include a seal 900 as also shown in FIG. 55 which extends into the central lumen 898 and which is configured to permit passage of the tenaculum 500 while providing a substantially fluid tight seal between an outside surface of the tenaculum 500 and an inside surface of the central lumen 898. For some embodiments, the seal 900 may be formed from a resilient flexible material and may include a small central hole 901 which passes through the seal 900. The small central hole 901 may be configured such that it expands about an outside surface of the tenaculum and forms a fluid tight seal. The morcellator 896 may further include a second seal 902 which is disposed such that it is substantially adjacent to the seal 900 within the central lumen 898. The second seal 902 may be configured with a duck billed configuration or any other suitable configuration which remains closed when it is not in use.

For some embodiments, as discussed above, the tenaculum 500 may further include the optical objective 908 which may be disposed on a distal section 954 of the tenaculum 500. The optical objective 908 may be configured to image in a distal direction along a longitudinal axis 907 of the tenaculum 500. In some cases, the optical objective 908 may be operatively coupled to a video display 911 and any other suitable imaging equipment in order to produce an image of the volume disposed distal of the tenaculum 500 that is viewable to a user outside the patient's body 31.

For some embodiments the morcellator cannula 894 may further include the insufflation lumen 892 as shown in FIGS. 55-56 and discussed above which may extend a length of the morcellator cannula 894. The insufflation lumen 892 may include an insufflation output port 893 which may be disposed at a distal end 976 of the morcellator cannula 894. In some cases, the insufflation lumen 892 may be configured such that it is in fluid communication via an insufflation tube, such as insufflation tube 940, with a suitable source of pressurized gas such as an insufflator 884 as shown in FIG. 55. The morcellator 896 may further include the locking member 974 which is disposed at the distal end 976 of the morcellator cannula 894. The locking member 974 may be configured as an expandable member that may be expanded from a collapsed state to an expanded state while deployed within a body cavity 30 of a patient 31. For some embodiments the locking member 974 may be configured as an inflatable balloon, and the morcellator cannula 894 may include a balloon inflation lumen 984 which extends the length of the morcellator cannula 894. The balloon inflation lumen 984 may also include a balloon inflation outlet port 986 which may be configured such that it is in fluid communication with an interior volume 988 of the inflatable balloon 974.

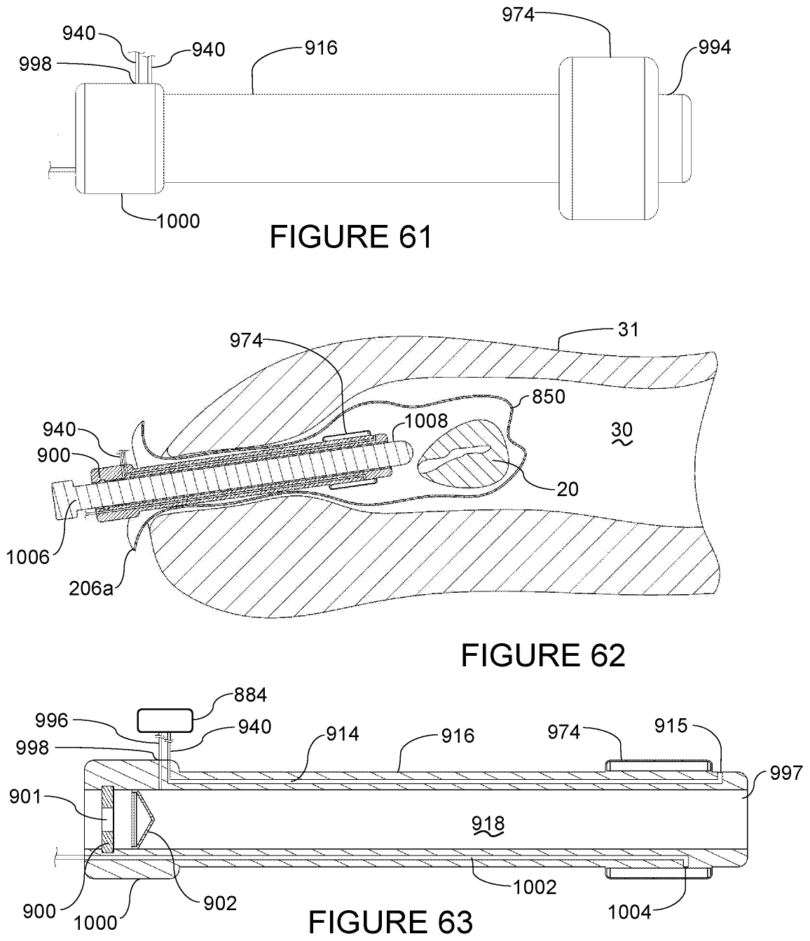

Some tissue containment and removal system embodiments 100 may include the access sheath 916 as shown in FIGS. 60-66 that includes a sheath cannula 990 which may be used in conjunction with a respective morcellator 400, 896 and container 200, 850 in order to perform a tissue containment and removal procedure or any other suitable procedure. Such a tissue containment and removal system embodiment 100 may include the container 850 which is configured to contain and isolate a tissue specimen 20. The container 850 may include an interior volume 204 and an opening 206 having an edge 206a. The container 850 may also include the distal portion 878 which is disposed opposite the opening 206, and a wall portion 852 which may be configured such that it is thin and flexible and may be formed from a fluid tight material. The access sheath 916 may include a proximal end 992, a distal end 994 and the central lumen 918 which extends axially through a length of the sheath cannula 990 as shown in FIG. 63. The access sheath 916 may further include the locking member 974 which is disposed on the distal end 994 of the sheath cannula 990.

Morcellator embodiments 400, 896 may be configured so as to be slidably disposable within the central lumen 918 of the sheath cannula 990. The morcellator 400, 896 may include a morcellator cannula 894, with a central lumen 898 extending an axial length of the morcellator cannula 894. The morcellator 400, 896 may further have a distal end 976 which is at least partially disposable within the opening and interior volume 204 of the container 850.

The central lumen 918 of the sheath cannula 990 may include a seal 900 which extends into the central lumen 918 and which is configured to permit passage of the morcellator 400, 896 while providing a substantially fluid tight seal between an outside surface of the morcellator 400, 896 and an inside surface of the central lumen 918 of the sheath cannula 990. For some embodiments the seal 900 may be formed from a resilient flexible material and may be configured with a small center hole 901 which passes through the seal 900. The small central hole 901 may be configured to expand about an outside surface of the morcellator 400, 896 while maintaining a substantially fluid tight seal. The central lumen 918 of the sheath cannula 990 may further include a second seal 902 which is disposed such that it is substantially adjacent to the seal 900 within the central lumen 918 of the sheath cannula 990. The second seal 902 may be formed from a resilient flexible material and may be configured as a duck bill or any other suitable configuration which remains closed when not in use.

The sheath cannula 990 may further include the insufflation lumen 914 which may extend a length of the sheath cannula 990 and which may be in fluid communication with an insufflation outlet port 915 which is disposed at the distal end 994 of the sheath cannula 990. For some embodiments, the insufflation lumen 914 may be configured such that it is in fluid communication with a source of pressurized gas such as an insufflator 884 as shown in FIG. 63. Tissue containment and removal system embodiments 100 may also include an optional insufflation lumen 996 as shown in FIG. 65 which may be formed between an inside surface of the sheath cannula 990 and an outside surface of the morcellator cannula 894 which is disposed within the sheath cannula 990. The optional insufflation lumen 996 may have an insufflation outlet port 997 at a distal end 994 of the sheath cannula 990 and an insufflation inlet port 998 which is disposed through a proximal housing 1000 of the access sheath 916. The inlet port 998 may be in fluid communication between the central lumen 918 of the sheath cannula 990 and a position exterior to the access sheath 916 to provide fluid communication between the lumen 996 and a source of pressurized gas 884.

The locking member 974 may be disposed on the distal end 994 of the access sheath 916, with the locking member 974 being configured as an expandable member that may be expanded from a collapsed state to an expanded state and collapsed from the expanded state to the collapsed state while deployed within a body cavity 30 of a patient 31. For some embodiments, the locking member 974 may be configured as an inflatable balloon and the sheath cannula 990 may include a balloon inflation lumen 1002 which extends the length of the sheath cannula 990. The balloon inflation lumen 1002 may also include a balloon inflation outlet port 1004 which may be configured such that it is in fluid communication with an interior volume 988 of the inflatable balloon 974.

The balloon inflation lumen 1002 may be disposed in fluid communication with a source of pressurized fluid such as a syringe, or the like, that may contain a gas, such as carbon dioxide, a liquid such as saline, or any other suitable fill material. Some such tissue containment and removal systems 100 may also include an obturator 1006 as shown in FIG. 62 which is configured to be slidably disposed within and substantially fill the central lumen 918 of the sheath cannula 990. The obturator 1006 may include a rounded atraumatic distal end 1008 which extends distally from the distal end 994 of the sheath cannula 990 during insertion into a patient's access conduit 22.

In use, some tissue containment and removal system embodiments 100 of FIGS. 1, 45-46 and any other suitable embodiments discussed herein may function as follows. A portion of the container 850 may be introduced into a patient's abdominal cavity 30 through the patient's vagina 32 as shown in FIG. 45. A tissue specimen 20 may then be placed into an interior volume 204 of the container 850. In some cases, this process may be facilitated by the use of surgical instruments 610 disposed within laparoscopic ports 862 and the patient's cavity 30 so as to manipulate the wall portion 852 of container 850 via the tabs 854 and/or tethers 858 with the surgical instruments 610 as shown in FIGS. 46, 47 and 51. The edge 206a of the container 850 may then be withdrawn from the abdominal cavity 30 through the vagina 32 such that the edge 206a defining an opening 206 in the container 850 is disposed outside the vagina 32 as shown in FIG. 46. The distal end 976 of the morcellator 896 may be introduced into the interior volume 204 of the container 850 through the vagina 32 as shown in FIG. 57. The interior volume 204 of the container 850 may then be insufflated by supplying pressurized gas, such as carbon dioxide, into an insufflation manifold lumen 890 of an insufflation manifold 876 which extends along a wall portion 852 from the edge 206a into the interior volume 204 of the container 850 towards a distal portion 878 of the container 850.

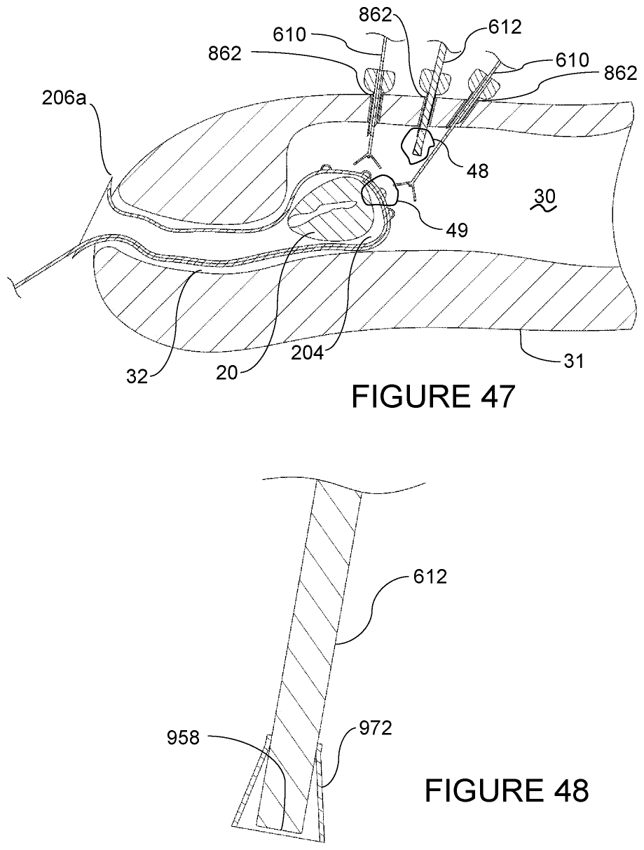

The manifold port 882 may be disposed in fluid communication between the insufflation manifold lumen 880 and the interior volume 204 of the container 850 as shown in FIGS. 29-30. As such, pressurized gas may then be emitted from at least one manifold port 882 of the insufflation manifold 876 into the interior volume 204 of the container 850 as shown in FIG. 47. The morcellator 896 may then be used to morcellate at least a portion of the tissue specimen 20 as shown in FIG. 58. The tissue specimen 20 may then be removed from the interior volume 204 of the container 850 and out of the vagina 32 through the central lumen 898 of the morcellator cannula 894.

In some cases, when morcellating at least a portion of the tissue specimen 20, a tenaculum 500 may be inserted through a central lumen898 of morcellator cannula 894 and the tissue specimen may then be grasped with the tenaculum 500 and proximally withdrawn into the blade 408 of the morcellator 896 with the tenaculum 500 as shown in FIG. 58. As the tissue specimen 20 is withdrawn into the morcellator blade 408, which may include a rotating blade 408, the tissue specimen 20 and the morcellation process may further be imaged by the optical objective 908 of the video imaging system 910. The optical objective 908 of the video imaging system 910 may be disposed on the distal end 954 of the tenaculum 500 and may also be operatively coupled to a video display 911 as shown in FIG. 55.

During such a tissue containment and removal procedure, it may be desirable for the interior volume of the abdominal cavity 30 outside of the container 850 to be insufflated. In some cases, the abdominal cavity 30 may be insufflated after the interior volume 204 of the container 850 has been insufflated. Additionally, a locking member 974 disposed on the distal end 976 of the morcellator 896 may be expanded from a collapsed state to an expanded state after the distal end 976 of the morcellator 896 has been introduced into the interior volume 204 of the container 850 through the vagina 32 as shown in FIG. 57. For some embodiments, the locking member 974 may be configured as an inflatable balloon and expanding the locking member may include inflating the balloon. In some cases, the balloon 974 may be inflated by pressurized gas through the balloon inflation lumen 984 disposed within the cannula 894 of the morcellator. After morcellation and tissue removal has been completed, the locking member 974 may be collapsed to a collapsed state prior to removal of the morcellator from the patient 31.

During the procedure, as discussed above, the wall portion 852 of the container 850 may be manipulated using an atraumatic grasper instrument 610 which may be disposed through a laparoscopic port 862 in the abdominal wall of the abdominal cavity 30 of the patient 31 as shown in FIGS. 51 and 57-59. In some cases, the atraumatic grasper 610 may have a distal end which may be releasably secured to a tab 854 which may be secured to and extend radially outward from an outer surface 856 of the container 850. In some cases, the atraumatic grasper 610 may have a distal end which may be releasably secured to a tether 858 that is secured to and extends from the tab 854 which may be secured to and extend radially outward from an outer surface 856 of the container 850. A portion of the tether 858 may be withdrawn through the laparoscopic port 862 in the abdominal wall of the abdominal cavity 30, and tension may be applied to the tether 858 from within the abdominal cavity 30 or once it is outside of the abdominal cavity 30.

In use, some tissue containment and removal system embodiments of FIGS. 55-59 may function as follows. A portion of the container 850 may be introduced into a patient's abdominal cavity 30 through the patient's vagina 32 as shown in FIG. 45. Tissue specimen 20 may then be placed into an interior volume 204 of the container 850 as discussed above. The edge 206a of the container 850 may then be withdrawn from the abdominal cavity 30 through the vagina 32 such that the edge 206a defining the opening 206 in the container 850 is disposed outside the vagina 32 as shown in FIG. 46. The distal end 976 of the morcellator 896 may then be introduced into the interior volume 204 of the container 850 through the vagina 32 as shown FIG. 57. The interior volume 204 of the container 850 may then be insufflated by supplying pressurized gas into the insufflation lumen 892 of a cannula 894 of the morcellator 896 which extends from the proximal end of the cannula 894 to a distal end 976 of the morcellator cannula 894. The pressurized gas may be emitted from an outlet port 893 which is in fluid communication between the insufflation lumen 890 and the interior volume 204 of the container 850. The morcellator 896 may then be used to morcellate at least a portion of the tissue specimen 20. The tissue specimen 20 may then be removed from the interior volume 204 of the container 850 and out of the vagina 32 through the central lumen 898 of the cannula 894 of the morcellator 896.

In use, some tissue containment and removal system embodiments 100 of FIGS. 60-66 may function as follows. A portion of the container 850 may be introduced into a patient's abdominal cavity 30 through the patient's vagina 32 as shown in FIG. 45. The tissue specimen 20 may then be placed into an interior volume 204 of the container 850 as discussed above. The edge 206a of the container 850 may then be withdrawn from the abdominal cavity 30 through the vagina 32 such that the edge 206a defining the opening 206 in the container 850 is disposed outside the vagina 32 as shown in FIG. 46. The distal end 994 of an access sheath 916 may then be introduced into the interior volume 204 of the container 850 through the vagina 32 as shown in FIG. 62. The interior volume 204 of the container 850 may then be insufflated by supplying a pressurized gas into an insufflation lumen 914 of the sheath cannula 990 of the access sheath 916. In some cases, the insufflation lumen 914 may extend from a proximal end 992 of the sheath cannula 990 to a distal end 994 of the sheath cannula 990 and the insufflation lumen 914 may have an insufflation outlet port 997 which is in fluid communication between the insufflation lumen 914 and the interior volume 204 of the container 850. The morcellator 896 may then be used to morcellate at least a portion of the tissue specimen 20. The tissue specimen 20 may then be removed from the interior volume 204 of the container 850 and out of the vagina 32 through the central lumen 898 of the cannula 894 of the morcellator 896.

Additionally, a locking member 974 disposed on the distal end 994 of the access sheath 916 and may be expanded from a collapsed state to an expanded state after the distal end 994 of the access sheath 916 has been introduced into the interior volume 204 of the container 850 through the vagina 32 as shown in FIG. 62. For some embodiments, the locking member 974 may be configured as an inflatable balloon and expanding the locking member 974 may include inflating the balloon. In some cases, the balloon 974 may be inflated by pressurized gas through the balloon inflation lumen 1002 disposed within the cannula 990 of the access sheath 916. After morcellation and tissue removal has been completed, the locking member 974 may be collapsed to a collapsed state prior to removal of the access sheath 916 from the patient 31.

As discussed above, in some instances it may be desirable to provide space within a container 850 between the tissue specimen 20 and the wall portion 852 of the container during tissue. In other cases, it may be desirable to use tension on the container 850 to minimize space within the interior volume 204 of the container 850 and use the tension on the wall portion 852 of the container 850 in order to bring the tissue specimen 20 into close proximity of a tissue cutter for the morcellation process. In such tissue containment and removal system embodiments 100, it may be useful to use components that minimize the risk of tearing or puncturing the container 850 during the procedure and also facilitate the morcellation process while also minimizing the amount of resources and personnel required to carry out such procedures.

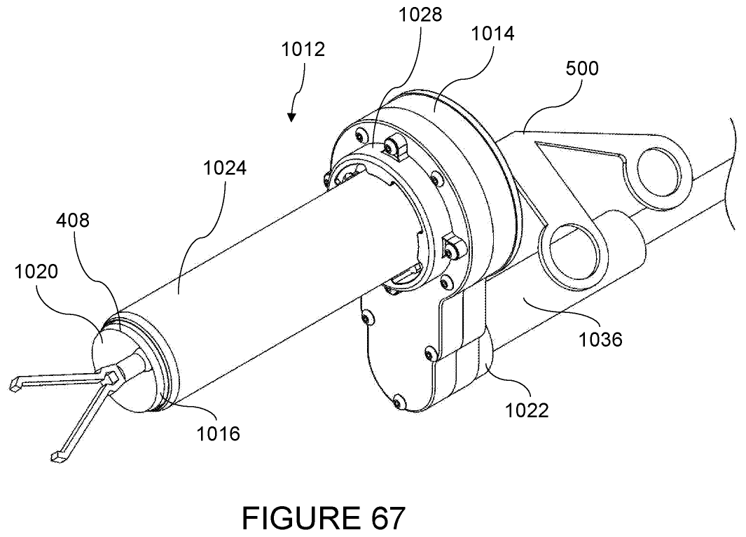

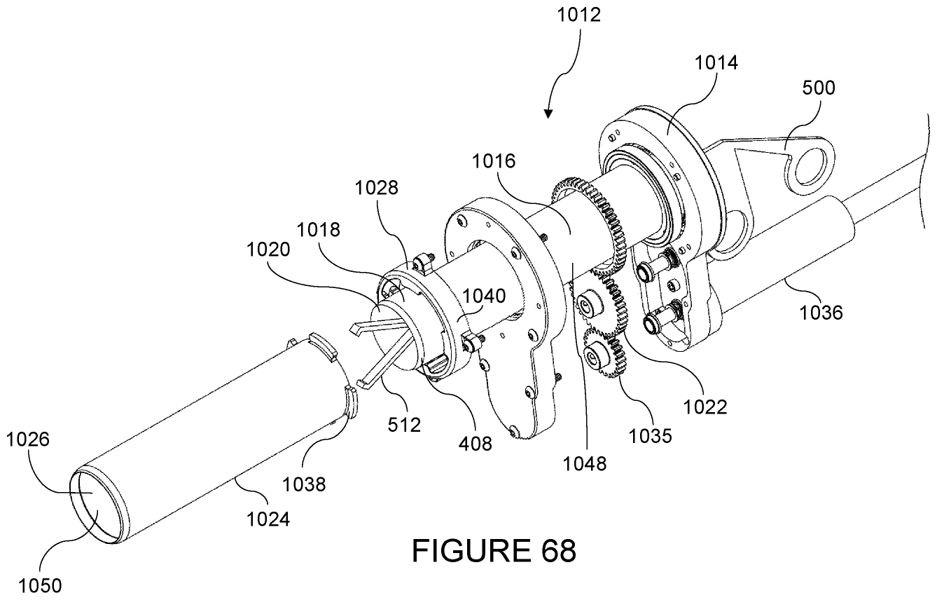

Referring to FIGS. 67-76, some embodiments of a tissue containment and removal system 1010 may include a tissue container 850 having an interior volume 204, and a wall portion 852 which may have a composite multiple layer structure that includes a first layer 872 made from a polymer material and a second layer 874 disposed inside of the first layer which includes a high strength cut and puncture resistant material. In some cases, the composite multiple layer structure of the wall portion 852 of the container 850 may include an optional third layer 870 (as shown in FIG. 30A) which is disposed inside of the second layer 874 and which may also include a polymer material similar to the first layer 872. Such a polymer material, which may be used for either or both of the first layer 872 and optional third layer 870 may be water tight and prevent the egress of biological cells and the like from within the interior volume of the container 850. Although the device and method embodiments illustrated in FIGS. 67-76 are shown with exemplary container embodiment 850, the device and method embodiments shown in these figures and discussed herein may be used with any suitable container embodiment 200, 850 etc. discussed herein. In particular, it should be noted that the process of insufflation of either the interior volume 204 of the container or insufflation of the body cavity 30 are optional, particularly with regard to the methods and devices shown in FIGS. 67-76.

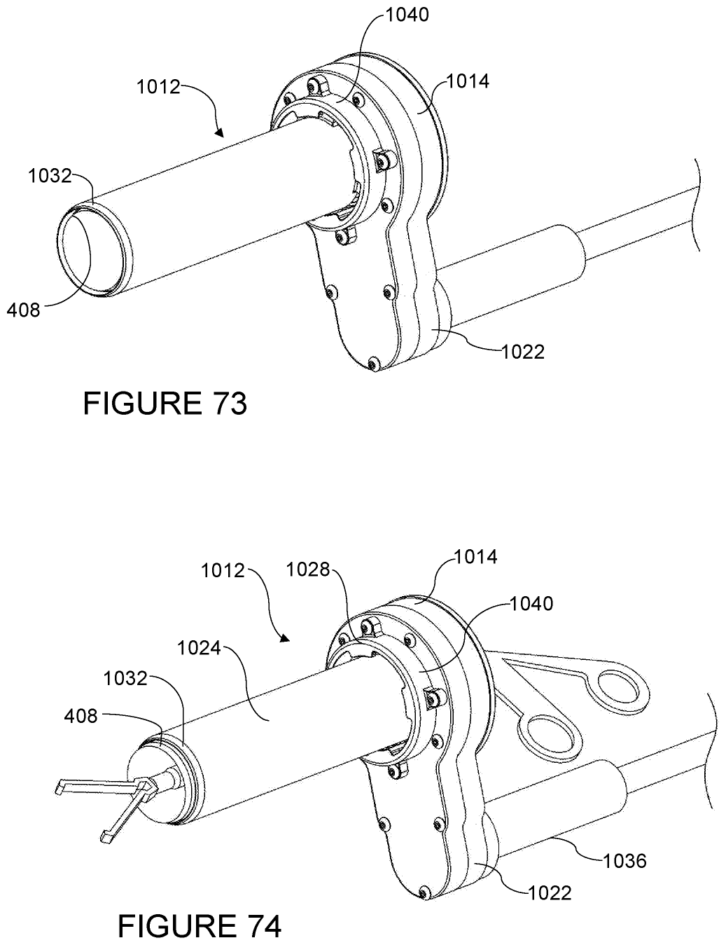

The system 1010 may also include a morcellator 1012 having a housing 1014 with a rigid configuration, a tissue cutter 1016 that has a tubular configuration, a distal end 1018, a central lumen 1020, a cutter blade 408 disposed on the distal end 1018, and an outer dimension that is at least partially disposable within the interior volume 204 of the container 850. The morcellator 1012 may also include a drive 1022 which is disposed within the housing 1014 and which is configured to rotate the tissue cutter 1016 relative to the housing 1014 when operated. The system 1010 may further include a cannula 1024 which has a tubular configuration, which is at least partially disposable within the interior volume 204 of the container 850 and which includes a central lumen 1026 that is configured for axial passage and rotation of the tissue cutter 1016. A releasable mount 1028 may also be optionally included with the system 101 which is configured to releasably secure a proximal end 1030 of the cannula 1024 to the housing 1014 in both a protected position with the distal end 1032 of the cannula 1024 extending distally over the cutter blade 408 with the cutter blade 408 being covered by the distal end 1032 of the cannula 1024 as shown in FIG. 73 and a cutting position with the distal end 1032 of the cannula 1024 disposed in a position which is disposed axially proximal of the cutter blade 408 with the cutter blade 408 exposed for tissue cutting and morcellation as shown in FIG. 74. In some cases, the distal end of the cutting blade 408 may extend distally about 2 mm to about 5 mm from the distal end 1032 of the cannula 1024 when the cannula 1024 is releasably secured in the cutting position as shown in FIG. 74. In general, the tissue containment and removal system embodiment 1010 and components thereof may have the same or similar features, dimensions and materials as those of any other suitable tissue containment and removal system embodiments 100 discussed herein.

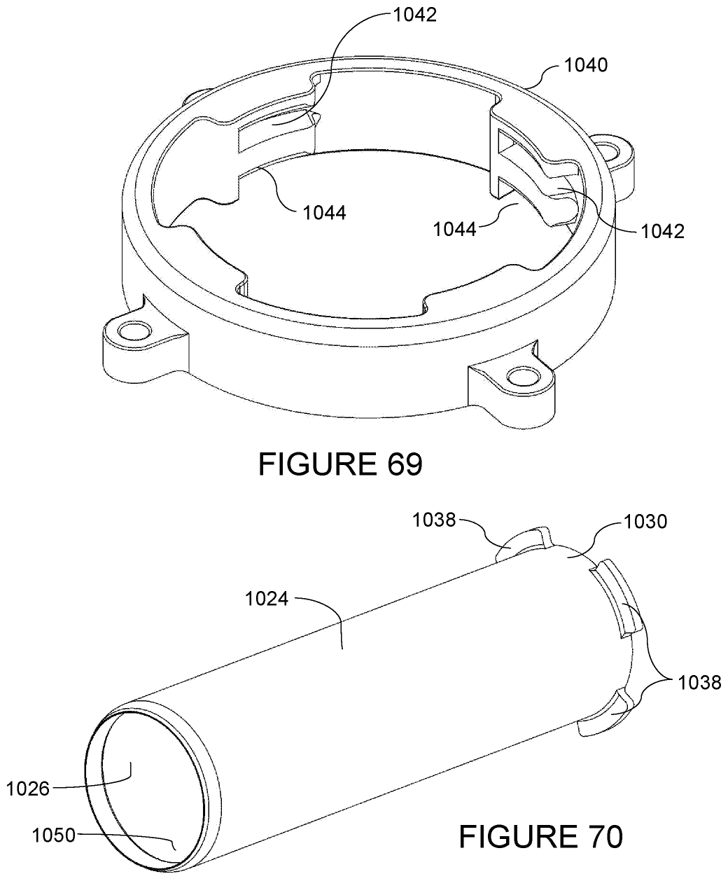

The drive 1022 may include a motorized drive with a motor 1034 configured to rotate the tissue cutter 1016 at about 30 rpm to about 500 rpm for some embodiments. Such drive embodiments 1022 may also include a transmission 1035 that includes a plurality of gears that transmit rotational energy from the motor 1034 to the tissue cutter 1016. In addition, the housing 1014 may further include a handle 1036 which may be used to facilitate gripping of the housing 1014 during insertion and operation of the drive 1022. For some system embodiments 1010, the releasable mount 1028 may include a bayonet type mount with at least one tab 1038 and a tab receiver 1040 including a first slot 1042 configured to releasably receive the tab 1038 while the cannula 1024 is in the protected position as shown in FIG. 69 and a second slot 1044 that is configured to releasably receive the tab 1038 while the cannula 1024 is in the cutting position. For the embodiment illustrated, the releasable mount 1028 comprises a plurality of tabs 1038 and a plurality of corresponding first slots 1042 and second slots 1044 with the tabs 1038 being secured to the proximal end 1030 of the cannula 1024 and the tab receiver 1040 being secured to the housing 1014. The cannula 1024 may be secured in the relative axial cutting position and protected position by suitably rotating the cannula 1024 about a longitudinal axis thereof such that the tabs 1038 are engaged or disengaged from the slots 1042 or 1044. For some embodiments of the morcellator 1012, the cannula 1024 may advanced and retracted from the cutting position and protected position with the use of a trigger mechanism (not shown) in place of the releasable mount 1028 shown.

In some instances, the second layer 874 of the composite multiple layer construction of the wall portion 852 of the container 850 as shown in FIG. 30A may include metal filaments 1046 in the form of the metal mesh structure 874. As discussed above, such a metal mesh layer 874 may have a thickness of about 0.0005 inches to about 0.004 inches, more specifically, about 0.0008 inches to about 0.002 inches, more specifically, a thickness of about 0.001 inches to about 0.0014 inches. For some embodiments, the metal mesh 874 may include a high strength and resilient material such as stainless steel, nickel titanium alloy, spring steel, copper or the like. In addition, the metal mesh of the middle layer 874 may include a mesh configuration having about 80 weaves per inch to about 600 weaves per inch, more specifically, about 300 weaves per inch to about 500 weaves per inch, and even more specifically, about 380 weaves per inch to about 420 weaves per inch. In some cases it may be desirable for the metal mesh structure of the middle layer to cover the entire expanse of the wall portion 852 of the container 850. For some container embodiments, the composite multiple layer wall portion 852 may be formed by heat rolling a polymer material such as polyurethane onto bare metal mesh fabric such that melted polymer material is melted through pores of the metal mesh and form a layer over each side of the metal mesh material sealing to the metal mesh material and optionally being mechanically captured by the pores of the metal mesh structure. In some instances, such container embodiments may further be formed by using a sheet of material such as described above, or any other flat sheet of material suitable for the wall portion 852, folded in half with a sealing of edges shaped to conform to the container embodiments 200, 850 discussed herein. Such containers 200, 850 may also be formed in three dimensions by blow molding, injection molding, compression molding processes or the like.

For some embodiments, the first layer 872 and optional third layer 870 may have a thickness of about 0.001 inches to about 0.01 inches, more specifically, about 0.002 inches to about 0.004 inches, even more specifically, about 0.0025 inches to about 0.0035 inches, and even more specifically, about 0.0028 inches to about 0.0032 inches.

In addition, for composite multiple layer structures of the wall portion 852, it may be desirable to maximize thermal insulation properties of one or more layers 870, 872, 874 of the wall portion 852. For example, thermally insulated materials such as polyimide or polytetrafluoroethylene can be used to better thermally insulate the container 850 from the surroundings. This configuration may be useful if the tissue cutter 1016 within the container 850 is generating significant amounts of heat, particularly with the use of alternative cutting techniques such as the use of radiofrequency energy ablation and the like. Such insulative properties may be useful to protect surrounding tissue of the cavity 30 from heat generated from the use of mono-polar radiofrequency energy, bi-polar radiofrequency energy, impedance based energy, harmonic energy or high frequency ultrasound energy. Such insulative properties may also be useful for electrically insulating an interior volume 204 of the container 850 from locations outside of the interior volume 204. As such, the first layer 872 and the third layer 870 of the composite multiple layer construction may, in some cases, include materials such as polyurethane, polyimide, polytetrafluoroethylene, nylon or the like. Polymer mesh materials such as nylon mesh may be used in any of the layer or layers of the wall portion 852 of the container 850 in conjunction with such materials to facilitate the insulative properties of the wall portion 852.

Electrically conductive properties of the wall portion 852 of the container 850 may also be useful in some circumstances. For example, a warning to a user indicating contact of the cutting blade 408 with the wall portion 852 might be generated from electrical continuity between the cutting blade 408 (or any other portion of cutting blade embodiments of morcellators discussed herein) and a conductive layer of the wall portion 852, such as the metal mesh layer 874. FIGS. 30C and 30D show a container embodiment 850 that includes an electrical connector 1056 which is electrically coupled the electrically conductive middle layer 874 by an electrical conduit 1058. Such an electrical connector 1056 may be electrically coupled to an electrical circuit that is also electrically coupled to the cutting blade 408 and that emits a warning when the cutting blade 408 makes electrical contact with the metal mesh 874.

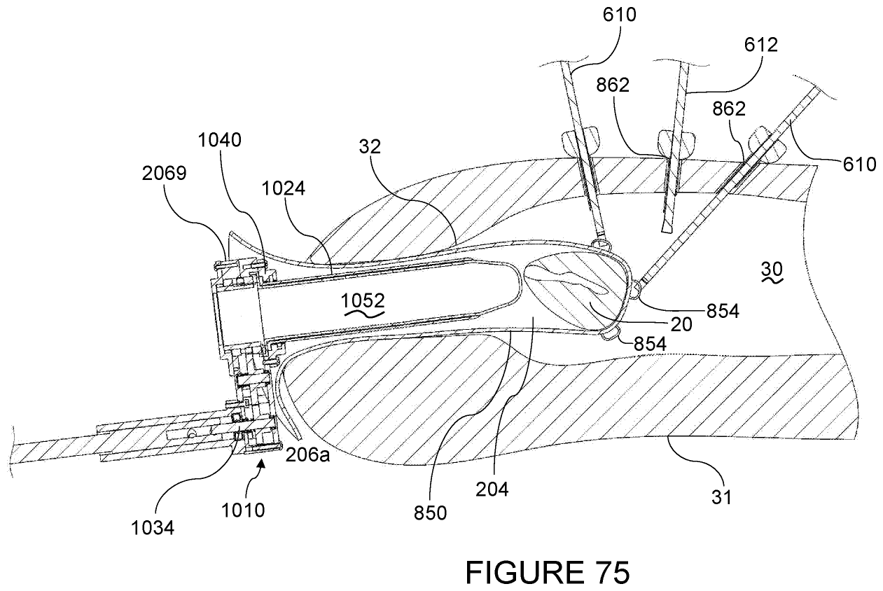

Some embodiments of the tissue containment and removal system 1010 may optionally include the tenaculum 500 which has jaws 512 configured for releasably securing tissue 20 disposed at a distal end thereof. Such a tenaculum embodiment 500 may be at least partially disposable within the interior volume 204 of the container 850 through the central lumen 1020 of the tissue cutter 1016. In addition, in some cases, the tenaculum 500 may further include a tenaculum stop 982 (as shown in FIG. 75 and discussed above) which is disposed on a proximal portion of a tenaculum shaft 906 of the tenaculum 500. The tenaculum stop 982 may be configured to limit a distance of extension of the jaws 512 of the tenaculum 500 from a cutting blade 408 of the tissue cutter 1016. For some embodiments, the tenaculum stop 1082 may be positioned on the tenaculum shaft 906 so as to limit the distal extension of the jaws 512 from the cutter blade 408 to a distance of up to about 30 mm.

In general, embodiments of the tissue cutter 1016 may have a generally tubular configuration made from a high strength rigid material such as stainless steel or the like. For some embodiments, an outside surface 1048 of the tissue cutter 1016 may be disposed in close proximity with an inside surface 1050 of the central lumen 1026 of the cannula 1024. For some embodiments, the tissue cutter 1016 may have an axial length of about 5 cm to about 30 cm, more specifically, about 8 cm to about 18 cm. In some cases, the tissue cutter 1016 may have an outer transverse dimension of about 10 mm to about 40 mm.

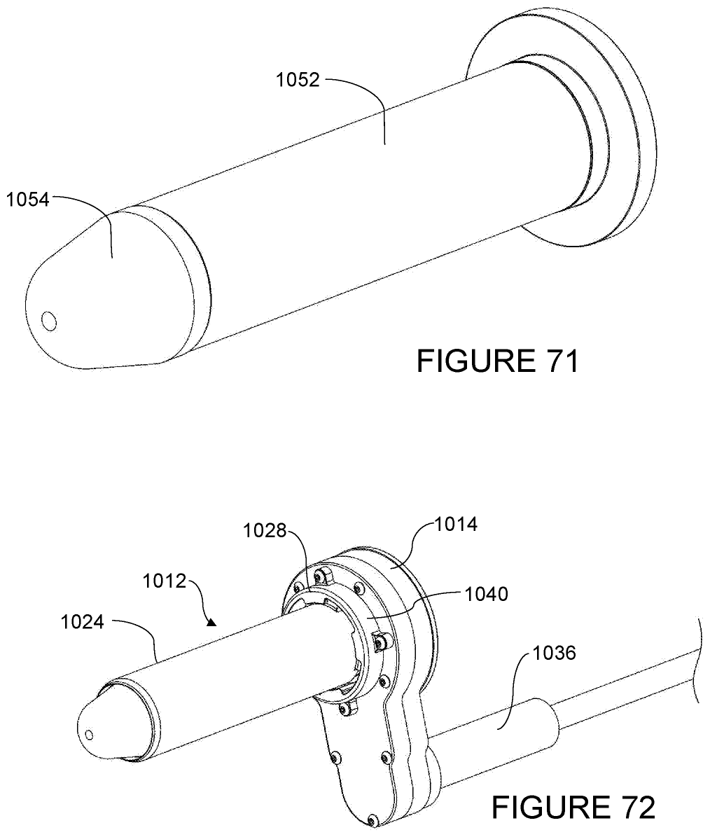

Some embodiments of the tissue containment and removal system 1010 include an optional obturator 1052 as shown in FIG. 71 which may have a generally cylindrical shape configured to be slidably disposed within the central lumen 1026 of the cannula 1024 or optionally the central lumen 1020 of the tissue cutter 1016. In some instances, an outer contour of the obturator 1052 may be sized so that an outer surface thereof is disposed in close proximity to the inner surface 1050 of the central lumen 1026 of the cannula 1024 or central lumen 1020 of the tissue cutter 1016. The obturator 1052 includes a rounded atraumatic distal end 1054 that extends distally from a distal end of either the cannula 1024 or tissue cutter 1016 for atraumatic insertion of the cannula 1024 or tissue cutter 1016 into the container 850 while the container 850 is deployed within the body cavity 30 of the patient 31. For some embodiments, the obturator 1052 may include a rigid biocompatible polymer such as nylon, ABS plastic, silicone, polytetrafluoroethylene, polyurethane or the like.

Some embodiments of a method of capturing and removing tissue transvaginally may include introducing at least a portion of the container 850 into the patient's pelvic cavity 30 through the vagina 32 of the patient 31. As discussed above, the container 850 may include an interior volume 204, a wall portion 852 having a composite multiple layer structure that includes a first layer 872 including a polymer and a second layer 874 disposed inside of the first layer 872 which includes a high strength cut and puncture resistant material. The tissue specimen 20 disposed within the patient's pelvic cavity 30 may be placed into the interior volume 204 of the container 850 and the edge 206a of the opening 206 of the container 850 removed from the pelvic cavity 30 through the vagina 32 such that the edge 206a surrounding the opening 206 in the container 850 is disposed outside the vagina 32 as shown in FIG. 75. In some instances, the tissue specimen 20 may include a uterus of the patient 31. Thereafter, the distal end 1032 of the cannula 1024, which has a tubular configuration and the central lumen 1026 that extends an axial length thereof, may be inserted into the interior volume 204 of the container 850 so as to provide a conduit into the interior volume 204 of the container 850 from outside the vagina 32. In some cases, the optional obturator 1052 may be inserted into the central lumen 1026 of the cannula 1024 until the rounded atraumatic distal end 1054 of the obturator 1052 extends distally from the distal end 1032 of the cannula 1024 prior to inserting the distal end 1032 of the cannula 1024 into the interior volume 204 of the container 850. After insertion of the cannula 1024 into the interior volume 204, the obturator 1052 may be removed.

The tissue cutter 1016 of the morcellator 1012 may then be inserted through the central lumen 1026 of the cannula 1024 and into the interior volume 204 of the container 850. The proximal end 1030 of the cannula 1024 may be releasably secured to the housing 1014 of the morcellator 1012 such that the axial position of the tissue cutter 1016 is substantially fixed with respect to an axial position of the cannula 1024. Such releasable securing of the proximal end 1030 of the cannula 1024 to the housing 1014 may take place either before or after insertion of the cannula 1024 into the interior volume 204 of the container 850. If the proximal end 1030 of the cannula 1024 is releasably secured to the housing 1014 prior to insertion of the cannula 1024 into the interior volume 204 of the container 850, the tabs 1038 may be positioned in the slots 1042 with the cannula 1024 in the protected position wherein the distal end 1032 of the cannula 1024 is disposed over the cutting blade 408 of the tissue cutter 1016 in order to protect the wall portion 852 of the container 850 during insertion. Once inserted, the tabs 1038 may be disengaged from the corresponding slots 1042 and re-engaged into the slots 1044 with the cannula 1024 in the cutting position with the cutting blade 408 extending distally of the distal end 1032 of the cannula 1024. Thereafter, at least a portion of the tissue specimen 20 may be cut with a cutter blade 408 of the tissue cutter 1016 by operating the optional motor 1034 and drive 1022 of the morcellator 1012, and the tissue specimen 20 removed from the interior volume 204 of the container 850 and out of vagina 32 through a central lumen 1020 of the tissue cutter 1016.

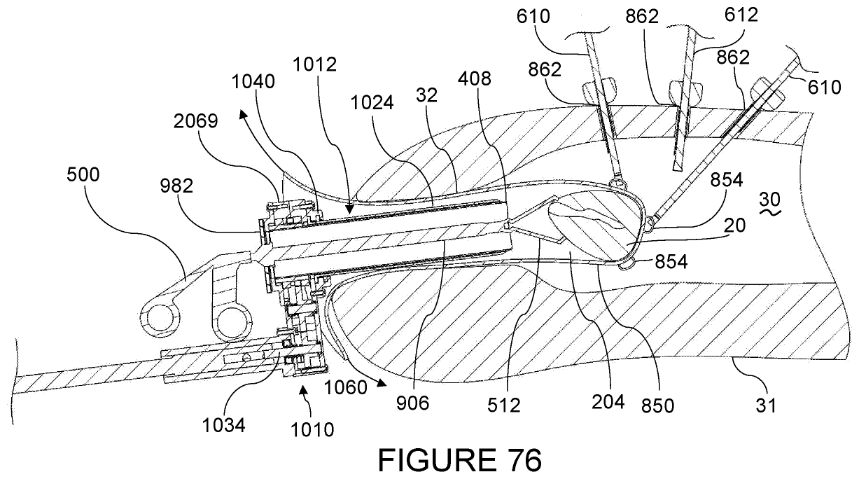

For some embodiments, tension may be applied to at least a portion of the tissue container 850 from a position outside the pelvic cavity 30 so as to bring the tissue specimen 20 into close proximity with the cutter blade 408 prior to or concurrently with cutting the at least one portion of the tissue specimen 20. In some cases, applying tension to at least a portion of the tissue container 850 includes physically applying tension on the tissue container 850 with the hand of an operator and more specifically may include rolling the edge 206a of the container 850 outside of the vagina 32 of the patient 31 as shown in FIG. 5C and discussed above, and as indicated by the arrows 1060 extended from the edge 206a of the container 850 in FIG. 76. Such tension may also be applied by mechanical mechanisms, such as mechanism 800 discussed above.

For some embodiments, cutting the tissue specimen 20 with the cutter blade 408 includes rotating the cutting blade 408. In some cases, as discussed above, the morcellator 1012 may include the drive 1022 which is disposed within the housing 1014 and which is configured rotate the tissue cutter 1016 relative to the housing 1014 when operated. Furthermore, the drive 1022 may include a motorized drive having a motor 1034 and operating the drive 1022 includes actuating the motor 1034 in order to rotate the cutting blade 408 relative to the housing 1014 and the tissue specimen 20 such that the cutting blade 408 rotates and moves relative to the tissue specimen 20 resulting in a dynamic slicing of the tissue specimen 20 which is in contact with the cutting blade 408. In some instances, the cutting blade 408 may be rotated with the motorized drive 1022 at about 30 rpm to about 500 rpm.

Further with regard to tissue cutting and removal, some method embodiments may optionally include introducing the tenaculum 500 at least partially into the interior volume 204 of the tissue container 850 through the central lumen 1020 of the tissue cutter 1016. The tenaculum 500, as shown in FIG. 75, may be used for grasping at least a portion of the tissue specimen 20 with jaws 512 of the tenaculum 500 and drawing the tissue specimen 20 into contact with the cutting blade 408 of the tissue cutter 1016 during tissue cutting with use of the tenaculum 500.

### Other Examples

The systems and components described in the present disclosure may be used in a variety of mammalian body locations. These systems and components are useful many circumstances, but especially those in which a relatively large tissue specimen must be safely removed through a relatively small port, whether that port be surgically created or a natural opening in the patient's body 31. Of course, the relative sizes of the systems and various components may be tailored to suit the specific application for which it is being used. For example, systems of the present disclosure used to capture and retrieve a tissue specimen, such as a cyst or tumor, from a lung via the trachea and perhaps accessed via the mouth or a nasal passage will be different than a system for a transvaginal hysterectomy. Moreover, components within a single system be sized differently relative to one another than respective components in a different system designed to treat a particular indication. We have found this to be a platform technology suitable for use in many applications. As such, each of the various examples described in the present disclosure should not be considered as limiting but rather exemplary of the platform concepts; modifications to relative dimensional recitations and departures from certain design features are considered to be within the scope of the present disclosure.

Gynecologic Applications

As described herein, systems 100 of the present disclosure are suitable for use in benign gynecological applications, including vaginal hysterectomy, laparoscopic assisted vaginal hysterectomy, laparoscopic hysterectomy, robotic assisted laparoscopic hysterectomy, and even open hysterectomy via a mini-laparotomy. A hysterectomy may include a full hysterectomy but also a partial hysterectomy such as a supra-cervical hysterectomy. In the case of a supra-cervical hysterectomy, some system embodiments discussed herein may be inserted into an abdominal incision rather than through the vagina. A supra-cervical hysterectomy may be performed also by inserting some system embodiments through a slit in between the posterior cervix and the posterior vagina. This technique may be similar to a posterior colpotomy in some instances. In the realm of laparoscopic and robotic assisted laparoscopic, the systems 100 of the present disclosure are equally useful in using any configurations of ports including three-port, four-port or five-port. In connection with such uses, any number or combination of cameras, device “arms,” and assistant ports are possible, although embodiments of the present disclosure may be used without such ports and their respective tools, especially those embodiments that can be introduced via a natural body port such as the vagina. The ability for systems of the present disclosure to work without an assistant port, necessary in other morcellation systems, renders systems disclosed herein as useful in “single site” robotic assisted hysterectomies as well.

Myomectomy: other embodiments of systems described herein may be deployed via an abdominal port for the purpose of morcellating uterine fibroids in connection with a myomectomy. Given that myomectomy procedures typically do not involve an incision such as a colpotomy, no component needs to be introduced vaginally.

Ovarian cystectomy, oophorectomy and salpingectomy: smaller, scaled-down versions of the embodiments of the present disclosure may be deployed via abdominal ports for the purpose of morcellating ovaries, fallopian tubes, ovarian cysts, tumors, or other solid or non-solid masses—tissues that otherwise would be difficult or impossible to remove via laparoscopic ports 862 or small transabdominal incisions using known devices and techniques.

Gynecologic oncology: embodiments of systems disclosed herein are suitable for performing hysterectomies associated with gynecologic malignancies or suspected gynecologic malignancies (including, e.g., uterine, ovarian, adnexal, cervical, omental and abdominal). Enclosure of the tissue specimen to be removed in containers disclosed herein prevents spillage of cancerous cells into the abdomen and pelvis.

Non-Gynecologic Applications

Embodiments of the present disclosure that are port-deployed, transabdominally deployed, transdermally deployed and deployed by other means are useful in various non-gynecologic procedures, including surgeries being performed for removing benign, malignant, or suspicious tissue in various locations in the body. Examples include bowel resection, colectomy, hemi-colectomy, as well as surgery for renal, adrenal, rectal, and bladder conditions. Head and neck surgery (including, e.g., surgery for thyroid conditions), cardiothoracic surgery (including, e.g., video assisted thoracic surgery (VATS) for pulmonary, cardiac, or other conditions) are other classes of treatment in which systems of the present disclosure are useful. Currently, minimally invasive procedures for these indications require that the patient 31 indeed receive a large, open incision for the purpose of liberating the specimen once it has been robotically dissected away from the body. Use of our bag/morcellator will both protect the specimen from spillage into the body cavity while simultaneously obviating the need for a large incision at the conclusion of the case—an incision which of course negates many of the advantages of minimally invasive surgery.

Features described herein with respect to different methods of use or different features, instruments, components, or their order of use may interchangeably be used among the various methods without taking away from the spirit of the methods and devices of the present disclosure. The presence or absence of a particular step or component should not be construed as limiting the methods described herein,

With regard to the above detailed description, like reference numerals used therein may refer to like elements that may have the same or similar dimensions, materials and configurations. While particular forms of embodiments have been illustrated and described, it will be apparent that various modifications can be made without departing from the spirit and scope of the embodiments discussed. Accordingly, it is not intended that the invention be limited by the foregoing detailed description.

The entirety of each patent, patent application, publication and document referenced herein is hereby incorporated by reference. Citation of the above patents, patent applications, publications and documents is not an admission that any of the foregoing is pertinent prior art, nor does it constitute any admission as to the contents or date of these documents.

Modifications may be made to the foregoing embodiments without departing from the basic aspects of the technology. Although the technology may have been described in substantial detail with reference to one or more specific embodiments, changes may be made to the embodiments specifically disclosed in this application, yet these modifications and improvements are within the scope and spirit of the technology. The technology illustratively described herein suitably may be practiced in the absence of any element(s) not specifically disclosed herein. Thus, for example, in each instance herein any of the terms “comprising,” “consisting essentially of,” and “consisting of” may be replaced with either of the other two terms. The terms and expressions which have been employed are used as terms of description and not of limitation, and use of such terms and expressions do not exclude any equivalents of the features shown and described or portions thereof, and various modifications are possible within the scope of the technology claimed. The term “a” or “an” may refer to one of or a plurality of the elements it modifies unless it is contextually clear either one of the elements or more than one of the elements is described. Although the present technology has been specifically disclosed by representative embodiments and optional features, modification and variation of the concepts herein disclosed may be made, and such modifications and variations may be considered within the scope of this technology.

Certain embodiments of the technology are set forth in the claims that follow.

1-68. (canceled)

69. A tissue containment and removal system, comprising:
a tissue container including an interior volume, and a wall portion having a composite multiple layer structure that comprises a first layer and a second layer disposed inside of the first layer which includes a high strength cut and puncture resistant material;
a morcellator comprising:
a housing having a rigid configuration,
a tissue cutter that has a tubular configuration, a distal end, a central lumen, a cutter blade disposed on the distal end, an outer dimension that is at least partially disposable within the interior volume of the tissue container, and
a drive which is configured to rotate the tissue cutter relative to the housing when operated;
a cannula which is at least partially disposable within the interior volume of the tissue container and which includes a central lumen that is configured for axial passage and rotation of the tissue cutter; and
wherein the second layer of the tissue container comprises an electrically conductive layer which is electrically coupled to an electric circuit that is also electrically coupled to the cutter blade, the electrical circuit being configured to emit a warning when the cutter blade makes electrical contact with the electrically conductive layer of the tissue container.

70-75. (canceled)

76. The tissue containment and removal system of claim 69 wherein the second layer of the composite multiple layer construction comprises metal filaments.

77. The tissue containment and removal system of claim 76 wherein the second layer of the composite multiple layer construction comprises a metal mesh.

78. The tissue containment and removal system of claim 77 wherein the metal mesh comprises a thickness of about 0.0005 inches to about 0.004 inches.

79. (canceled)

80. The tissue containment and removal system of claim 77 wherein the metal mesh comprises a material selected from the group consisting of stainless steel, nickel titanium alloy, and spring steel.

81. The tissue containment and removal system of claim 69 wherein the first layer of the composite multiple layer construction comprises a material selected from the group consisting of polyethylene, poly-paraphenylene terepthalamide, polyurethane, polyimide, polytetrafluoroethylene and nylon.

82. The tissue containment and removal system of claim 81 wherein the first layer comprises a thickness of about 0.001 inches to about 0.010 inches.

83. (canceled)

84. The tissue containment and removal system of claim 81 wherein the composite multiple layer structure of the wall portion of the container comprises a third layer which is disposed inside of the second layer and which comprises a polymer.

85. The tissue containment and removal system of claim 84 wherein the third layer of the composite multiple layer construction comprises a material selected from the group consisting of polyethylene, poly-paraphenylene terepthalamide, polyurethane, polyimide, polytetrafluoroethylene and nylon.

86. The tissue containment and removal system of claim 85 wherein the third layer comprises a thickness of about 0.001 inches to about 0.010 inches.

87. (canceled)

88. The tissue containment and removal system of claim 69 further comprising a tenaculum which comprises jaws configured for releasably securing tissue disposed at a distal end thereof and which is at least partially disposable within the interior volume of the tissue container through the central lumen of the tissue cutter.

89. The tissue containment and removal system of claim 88 wherein the tenaculum further comprises a tenaculum stop disposed on a proximal portion of a tenaculum shaft of the tenaculum and which is configured to limit the distance of extension of the jaws from a blade of the tissue cutter.

90. The tissue containment and removal system of claim 89 wherein the tenaculum stop is positioned on the tenaculum shaft so as to limit the extension of the jaws from the blade to a distance of up to about 30 mm.

91. The tissue containment and removal system of claim 69 wherein an outside surface of the tissue cutter is disposed in close proximity with an inside surface of the central lumen of the cannula.

92-98. (canceled)

99. The tissue containment and removal system of claim 69 further comprising an obturator which is configured to be slidably disposed within the central lumen of the tissue cutter of the morcellator with an outer surface of the obturator disposed in close proximity to an inner surface of the central lumen of the tissue cutter and which includes a rounded atraumatic distal end that extends distally from a distal end of the tissue cutter for atraumatic insertion of the tissue cutter into the container.

100-101. (canceled)

102. A method of capturing and removing tissue transvaginally, comprising:
introducing at least a portion of a container into a patient's pelvic cavity through a vagina of a patient, the container comprising an interior volume, a wall portion having a composite multiple layer structure that includes a first layer comprising a polymer and a second layer disposed inside of the first layer, the second layer comprising an electrically conductive layer of a high strength cut and puncture resistant material;
placing a tissue specimen into the interior volume of the container;
removing an edge of an opening of the container from the pelvic cavity through the vagina such that the edge surrounding the opening in the container is disposed outside the vagina;
inserting a distal end of a cannula which has a tubular configuration and a central lumen that extends an axial length thereof into the interior volume of the container so as to provide a conduit into the interior volume of the container from outside the vagina;
inserting a tissue cutter of a morcellator through the central lumen of the cannula and into the interior volume of the container;
emitting a warning from an electrical circuit that is electrically coupled to the electrically conductive layer and a cutter blade of the tissue cutter when the cutter blade makes electrical contact with the electrically conductive layer;
cutting at least a portion of the tissue specimen with the cutter blade of the tissue cutter; and
removing the tissue specimen from the interior volume of the container and out of vagina through a central lumen of the tissue cutter.

103. The method of claim 102 further comprising inserting an obturator into the central lumen of the cannula until a rounded atraumatic distal end of the obturator extends distally from a distal end of the cannula prior to inserting the distal end of the cannula into the interior volume of the container.

104. The method of claim 103 wherein inserting the obturator into the central lumen of the cannula comprises inserting the obturator which is configured to be slidably disposed within the central lumen of the cannula with an outer surface of the obturator disposed in close proximity to an inner surface of the central lumen of the cannula.

105. The method of claim 102 further comprising applying tension to at least a portion of the tissue container from a position outside the pelvic cavity so as to bring the tissue specimen into close proximity with the cutter blade prior to or concurrently with cutting the at least one portion of the tissue specimen.

106-108. (canceled)

109. The method of claim 102 wherein the morcellator further comprises a drive which is disposed within the housing and which is configured rotate the tissue cutter relative to the housing when operated and wherein cutting at least a portion of the tissue specimen with a cutter blade of the tissue cutter comprises operating the drive and rotating the cutter blade while pulling on the tissue specimen.

110. The method of claim 109 wherein the drive comprises a motorized drive having a motor and operating the drive comprises operating the motor to rotate the cutter blade.

111. The method of claim 110 wherein rotating the cutter blade comprises rotating the cutter blade with the motorized drive at about 30 rpm to about 500 rpm.

112. The method of claim 102 further comprising introducing a tenaculum at least partially into the interior volume of the tissue container through the central lumen of the tissue cutter and grasping at least a portion of the tissue specimen with jaws of the tenaculum.

113. (canceled)

114. The method of claim 102 wherein placing the tissue specimen into the interior volume of the tissue container comprises placing the patient's uterus into the interior volume of the tissue container.

115. The tissue containment and removal system of claim 69 further comprising a releasable mount that releasably secures a proximal end of the cannula to the housing in both a protected position with the distal end of the cannula extending distally over the cutter blade with the cutter blade being covered by the distal end of the cannula and a cutting position with the distal end of the cannula disposed in a position which is axially proximal of the cutter blade with the cutter blade exposed for tissue cutting and morcellation.

116. The tissue containment and removal system of claim 88 wherein the tenaculum further comprises an optical objective disposed on a distal section of the tenaculum and which is configured to image in a distal direction along a longitudinal axis of the tenaculum.

117. The tissue containment and removal system of claim 116 wherein the optical objective is operatively coupled to a video display.

118. The method of claim 102 further comprising releasably securing a proximal end of the cannula to a housing of the morcellator such that the axial position of the tissue cutter is substantially fixed with respect to an axial position of the cannula.
